(19) United States
(12) Patent Application Publication
(10) Pub. No.: US 2022/0135539 A1
(43) Pub. Date: May 5, 2022

(54) BIARYL AMIDE COMPOUNDS, PREPARATION METHODS AND MEDICAL APPLICATIONS THEREOF

(71) Applicant: Eternity Bioscience Inc., Cranbury, NJ (US)

(72) Inventors: Peng Zhao, Jamesburg, NJ (US); Heping Wu, Edison, NJ (US); Linghang Zhuang, Chalfont, PA (US); Yu Zhou, East Brunswick, NJ (US); Suxing Liu, Edison, NJ (US); Jian Liu, Edison, NJ (US)

(21) Appl. No.: 17/517,100

(22) Filed: Nov. 2, 2021

Related U.S. Application Data
(60) Provisional application No. 63/143,019, filed on Jan. 28, 2021.
(60) Provisional application No. 63/108,582, filed on Nov. 2, 2020.

(51) Int. Cl. C07D 401/12; C07D 405/12; C07D 213/64; C07D 405/14

(52) U.S. Cl. CPC C07D 401/12; C07D 405/14; C07D 213/64; C07D 405/12

(57) ABSTRACT

This application discloses RAF inhibitors of the general formula (I) and analogs thereof, pharmaceutical compositions containing these compounds, methods of preparing them, and use of these compounds as therapeutic agents for the treatment of various disorders related to the excessive RAF activity, including cancers.

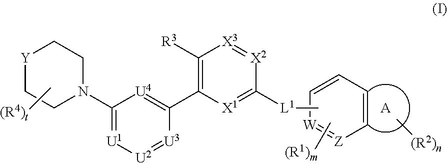

### CROSS-REFERENCE TO RELATED APPLICATIONS

This application claims priority under 35 U.S.C. § 119(e) to United States Provisional Patent Application No. 63/108,582, filed on Nov. 2, 2020, and Provisional Patent Application No. 63/143,019, filed on Jan. 28, 2021, the disclosures of both of which are incorporated herein by reference in their entirety.

### FIELD OF THE DISCLOSURE

The present disclosure relates to compounds and methods useful in the suppression of Rapidly Accelerated Fibrosarcoma (RAF), in particular use of the compounds and pharmaceutically acceptable compositions containing the compounds for the treatment of various disorders related to the excessive RAF activity, including cancers.

### BACKGROUND OF THE DISCLOSURE

The receptor tyrosine kinases, RAF, named for Rapidly Accelerated Fibrosarcoma, are a family of serine/threonine-specific protein kinases, from the TKL (Tyrosine-kinase-like) kinase group. There are three known mammalian Raf isoforms: A, B and C-Raf (also named Raf-1). Raf kinases participate in the RAS-RAF-MEK-ERK signal transduction cascade in the mitogen-activated protein kinase (MAPK) cascade (Rapp U. R., et al., Proc. Natl. Acad. Sci. USA, 1983, 80, 4218-4222; Sutrave P., et al., Nature, 1984, 309, 85-88; Moelling K., et al., Nature, 1984, 312, 558-561). The Ras-Raf-MAPK pathway controls various cellular physiological processes by transmitting signals from membrane-bound receptors to a large variety of membrane-based, cytoplasmic, and nuclear targets, coordinating diverse cellular responses. Extensive research demonstrated RAF family kinases playing pivotal roles in regulation of cell survival, proliferation and differentiation, apoptosis and many other physiological processes signaling though the MAPK cascade (Lavoie H., et al., Nat. Rev. Mol. Cell Biol., 2015, 16, 281-298). Aberrations along the Ras-Raf-MAPK pathway are involved in various biological processes related to human diseases. Excessive activity of the Ras-Raf-MAPK pathway components is a common mechanism in proliferative diseases, such as cancer (Wellbrock C., et al., Nat. Rev. Mol. Cell. Biol., 2004, 5, 875-885; Leicht D. T., et al., Biochim. Biophys. Acta, 2007, 1773, 1196-1212; Dhillon A. S., et al., Oncogene, 2007, 26, 3279-3290).

Given the strong correlation of RAF activity with cancers, RAF has been exploited as a target site for drug discovery as cancer therapies. A broad set of ATP-competitive RAF inhibitors (PLX4032/Vemurafenib, Dabrafenib, Sorafenib, etc.) have been approved for treating metastatic melanoma patients and demonstrated positive clinical efficacy against melanomas harboring the recurrent BRAFV600E allele (Holderfield, M., et al., Nat. Rev. Cancer, 2014, 14, 455-467; Chapman, P. B., et al., N. Engl. J. Med., 2011, 364, 2507-2516; Hauschild, A., et al., Lancet, 2012, 380, 358-365). Unfortunately, acquired resistance to these agents invariably develops in part by mechanisms that stimulate RAF dimerization, although the structural basis for this currently remains elusive. Concurrently, tumors exhibiting RAS activity owing to hyperactive RAS mutations but with wild type for BRAF, show primary resistance to these first-generation RAF inhibitors. In contrast, RAF inhibitors were found to induce ERK signaling in conditions where RAS activity is elevated and therefore enhanced tumor cell proliferation. This counterintuitive phenomenon, known as the paradoxical effect, was also observed in normal tissues relying on physiological RAS activity and is the basis for some of the adverse effects seen with RAF inhibitors in melanoma patients. The underlying mechanism results in part from the compound ability to promote kinase domain dimerization (Poulikakos, P. I., et al., Nature, 2011, 480, 387-390; Hatzivassiliou, G., et al., Nature, 2010, 464, 431-435; Heidorn, S. J., et al., Cell, 2010, 140. 209-221; Poulikakos, P. I., et al., Nature, 2010, 464, 427-430). Overall, the developed RAF inhibitors that are efficacious against BRAFV600E mutated tumors may not be as effective in tumors harboring RAS alterations.

### SUMMARY OF THE DISCLOSURE

To circumvent the limitation of the first-generation RAF inhibitors, the present disclosure provides compounds and methods useful in the suppression of RAF kinases, and use of these compounds to treat disorders related to hyperactive RAF, including cancers. The compounds are suited for treatment of tumors with aberrant MAPK pathway, including RAF and/or RAS mutations or alterations.

The present disclosure, in one aspect, provides a compound of formula (I) having the following structure:

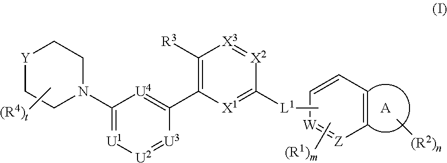

or a tautomer, racemate, enantiomer, diastereomer thereof, or mixture thereof, or a pharmaceutically acceptable salt, solvate or prodrug thereof, wherein:

ring A is an optionally substituted cycloalkyl or heterocyclyl fused to the adjacent aromatic ring;

W and Z are identical or different, and each is independently C or N, provided that W and Z are not both N at the same time;

R¹ at each occurrence is identical or different, and each is independently selected from hydrogen, hydroxyl, halogen, alkyl, alkenyl, alkynyl, haloalkyl, hydroxyalkyl, cyano, amino, —NHR⁶, —N(R⁶)₂, —OR⁷, —SO₂R⁸, —S(═NH)(═O)R⁸, cycloalkyl, heterocyclyl, aryl and heteroaryl;

R² at each occurrence is identical or different, and each is independently selected from hydrogen, hydroxyl, halogen, alkyl, alkenyl, alkynyl, haloalkyl, hydroxyalkyl, cyano, amino, —NHR⁶, —N(R⁶)₂, —SO₂R⁸, —S(═NH)(═O)R⁸, cycloalkyl, heterocyclyl, aryl and heteroaryl;

L₁ is —C(═O)—NR⁰— or —NR⁰—C(═O)—, wherein R⁰ is selected from hydrogen, alkyl, haloalkyl and hydroxyalkyl;

X¹, X² and X³ are identical or different, and each is independently selected from CH, N and NO;

R³ is selected from hydrogen, hydroxyl, halogen, alkyl, alkoxy, haloalkyl, hydroxyalkyl, cyano and amino;

U¹, U², U³ and U⁴ are identical or different, and each is independently CR⁵ or N, wherein R⁵ at each occurrence is independently selected from hydrogen, halogen, alkyl, alkenyl, alkynyl, alkoxy, hydroxyl, cyano, amino, —O-L₂-OH, —NHR⁶, —N(R⁶)₂, —SO₂R⁸, —NHSO₂R⁸, —NHC(O)R⁸, —NR⁶CO₂R⁸, cycloalkyl, heterocyclyl, aryl, heteroaryl, —O-cycloalkyl, —O-heterocyclyl, —O-aryl and —O-heteroaryl; wherein the alkyl, alkoxy, cycloalkyl, heterocyclyl, aryl and heteroaryl are each optionally substituted with one or more, preferably one to five, and sometimes more preferably one to three, groups independently selected from oxo, hydroxyl, halogen, alkyl, haloalkyl, hydroxyalkyl, cyano, amino and alkoxy; and wherein L₂ is alkylene;

Y is selected from O, S, S(═O) and SO₂;

R⁴ at each occurrence is identical or different, and each is selected from hydrogen, hydroxyl, halogen, alkyl, alkoxy, oxo, haloalkyl, hydroxyalkyl, cyano and amino;

R⁶, R⁷ and R⁸ at each occurrence are identical or different, and each is independently selected from alkyl, alkenyl, alkynyl, haloalkyl, hydroxyalkyl, cycloalkyl, heterocyclyl, aryl and heteroaryl;

m is 0, 1 or 2;

n is 0, 1, 2, 3 or 4; and

t is 0, 1, 2, 3 or 4.

In another aspect, the present disclosure provides a pharmaceutical composition, comprising a compound of formula (I), or a tautomer, racemate, enantiomer, diastereomer thereof, or mixture thereof, or a pharmaceutically acceptable salt, solvate or prodrug thereof, and one or more pharmaceutically acceptable carriers, diluents and/or other excipients.

In another aspect, the present disclosure provides a method for inhibiting RAF, comprising a step of administering to a subject in need thereof a therapeutically effective amount of a compound of formula (I), or a tautomer, racemate, enantiomer, diastereomer thereof, or mixture thereof, or a pharmaceutically acceptable salt, solvate or prodrug thereof, or the pharmaceutical composition thereof.

In another aspect, the present disclosure provides a method for treating a RAF-mediated disease or disorder, comprising a step of administering to a subject in need thereof a therapeutically effective amount of a compound of formula (I), or a tautomer, racemate, enantiomer, diastereomer thereof, or mixture thereof, or a pharmaceutically acceptable salt, solvate or prodrug thereof, or the pharmaceutical composition thereof; preferably, wherein the RAF-mediated disease or disorder is cancer.

The RAF-mediated diseases or disorder includes, but is not limited to, lymphoma, leukemia, breast cancer, lung cancer, prostate cancer, ovarian cancer, liver cancer, melanoma, rhabdomyosarcoma, synovial sarcoma, mesothelioma, cervical cancer, colon cancer, rectal cancer, stomach cancer, pancreatic cancer, brain cancer, skin cancer, oral cancer, bone cancer, kidney cancer, bladder cancer, fallopian tube tumor, ovarian tumor, peritoneal tumor, glioma, glioblastoma, head and neck cancer, and myeloma; preferably lymphoma, leukemia, breast cancer, lung cancer, prostate cancer, ovarian cancer, liver cancer, melanoma, rhabdomyosarcoma, synovial sarcoma and mesothelioma.

Other aspects and advantages of the present disclosure will be better appreciated in view of the following detailed description, examples, and claims.

### DETAILED DESCRIPTION OF THE DISCLOSURE

In one aspect, the present disclosure provides a compound of formula (I):

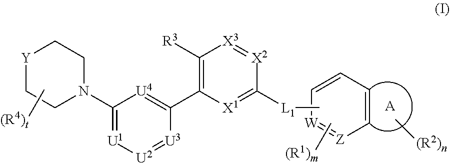

or a tautomer, racemate, enantiomer, diastereomer thereof, or mixture thereof, or a pharmaceutically acceptable salt, solvate or prodrug thereof, wherein:

ring A is an optionally substituted cycloalkyl or heterocyclyl fused to the adjacent aromatic ring;

W and Z are identical or different, and each is independently C or N, provided that W and Z are not both N at the same time;

R¹ at each occurrence is identical or different, and each is independently selected from hydrogen, hydroxyl, halogen, alkyl, alkenyl, alkynyl, haloalkyl, hydroxyalkyl, cyano, amino, —NHR⁶, —N(R⁶)₂, —SO₂R⁸, —S(═NH)(═O)R⁸, cycloalkyl, heterocyclyl, aryl and heteroaryl;

R² at each occurrence is identical or different, and each is independently selected from hydrogen, hydroxyl, halogen, alkyl, alkenyl, alkynyl, haloalkyl, hydroxyalkyl, cyano, amino, —NHR⁶, —N(R⁶)₂, —OR⁷, —SO₂R⁸, —S(═NH)(═O)R⁸, cycloalkyl, heterocyclyl, aryl and heteroaryl;

L₁ is —C(═O)—NR⁰— or —NR⁰—C(═O)—, wherein R⁰ is selected from hydrogen, alkyl, haloalkyl and hydroxyalkyl;

X¹, X² and X³ are identical or different, and each is independently selected from CH, N and NO;

R³ is selected from hydrogen, hydroxyl, halogen, alkyl, alkoxy, haloalkyl, hydroxyalkyl, cyano and amino;

U¹, U², U³ and U⁴ are identical or different, and each is independently CR⁵ or N, wherein R⁵ at each occurrence is independently selected from hydrogen, halogen, alkyl, alkenyl, alkynyl, alkoxy, hydroxyl, cyano, amino, —O-L₂-OH, —NHR⁶, —N(R⁶)₂, —SO₂R⁸, —NHSO₂R⁸, —NHC(O)R⁸, —NR⁶CO₂R⁸, cycloalkyl, heterocyclyl, aryl, heteroaryl, —O-cycloalkyl, —O-heterocyclyl, —O-aryl and —O-heteroaryl; wherein the alkyl, alkoxy, cycloalkyl, heterocyclyl, aryl and heteroaryl are each optionally substituted with one or more, preferably one to five, and sometimes more preferably one to three, groups independently selected from oxo, hydroxyl, halogen, alkyl, haloalkyl, hydroxyalkyl, cyano, amino and alkoxy; and wherein L₂ is alkylene;

Y is selected from O, S, S(═O) and SO₂;

R⁴ at each occurrence is identical or different, and each is selected from hydrogen, hydroxyl, halogen, alkyl, alkoxy, oxo, haloalkyl, hydroxyalkyl, cyano and amino;

R⁶, R⁷ and R⁸ at each occurrence are identical or different, and each is independently selected from alkyl, alkenyl, alkynyl, haloalkyl, hydroxyalkyl, cycloalkyl, heterocyclyl, aryl and heteroaryl;

m is 0, 1 or 2;

n is 0, 1, 2, 3 or 4; and

t is 0, 1, 2, 3 or 4.

In one embodiment, the disclosure provides a compound of formula (I), or a tautomer, racemate, enantiomer, diastereomer thereof, or mixture thereof, or a pharmaceutically acceptable salt, solvate or prodrug thereof, wherein Y is O.

In one embodiment, the disclosure provides a compound of formula (I), or a tautomer, racemate, enantiomer, diastereomer thereof, or mixture thereof, or a pharmaceutically acceptable salt, solvate or prodrug thereof, wherein ring A is a C₄-C₆ cycloalkyl or 4- to 6-membered heterocyclyl fused to the adjacent aromatic ring. In some embodiments, the heterocyclyl comprises one or two oxygen atoms in the ring.

In one embodiment of the disclosure, the compound of formula (I) is selected from a compound of formula (II):

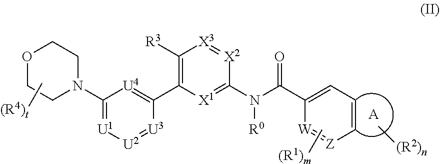

or a tautomer, racemate, enantiomer, diastereomer thereof, or mixture thereof, or a pharmaceutically acceptable salt, solvate or prodrug thereof, wherein:

W, Z, R⁰, X¹, X², X³, U¹, U², U³, U⁴, ring A, R¹ to R⁴, m, n and t are as defined in formula (I).

In one embodiment of the disclosure, the compound of formula (I) is selected from a compound of formula (III):

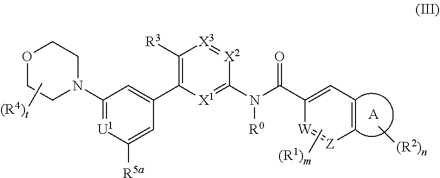

or a tautomer, racemate, enantiomer, diastereomer thereof, or mixture thereof, or a pharmaceutically acceptable salt, solvate or prodrug thereof, wherein:

R^{5a} is —O-L₂-OH; preferably R^{5a} is —O—(CH₂)₁₋₆—OH; more preferably R^{5a} is —O—(CH₂)₂—OH;

L₂ is alkylene; preferably L₂ is C₁₋₆ alkylene; more preferably L₂ is CH₂; and

W, Z, R⁰, X¹, X², X³, U¹, ring A, R¹ to R⁴, m, n and t are as defined in formula (I).

In one embodiment, the disclosure provides a compound of formula (I), or a tautomer, racemate, enantiomer, diastereomer thereof, or mixture thereof, or a pharmaceutically acceptable salt, solvate or prodrug thereof, wherein

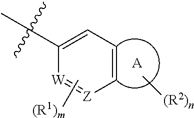

is selected from

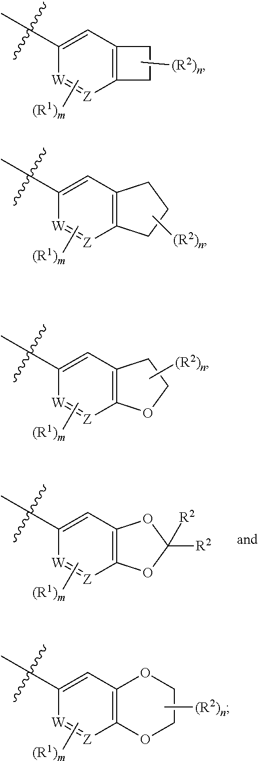

preferably

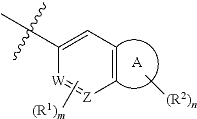

is selected from

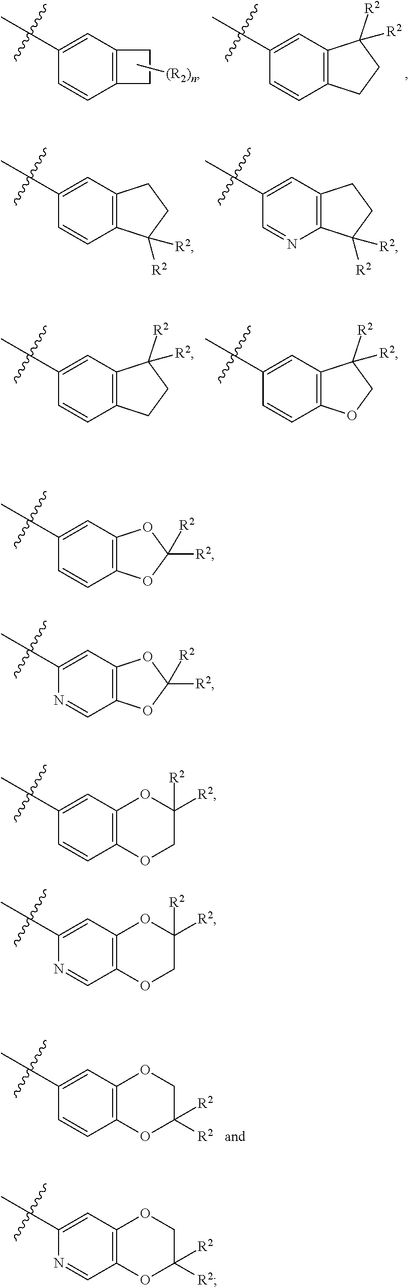

more preferably

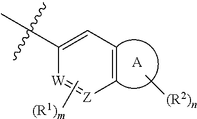

is selected from

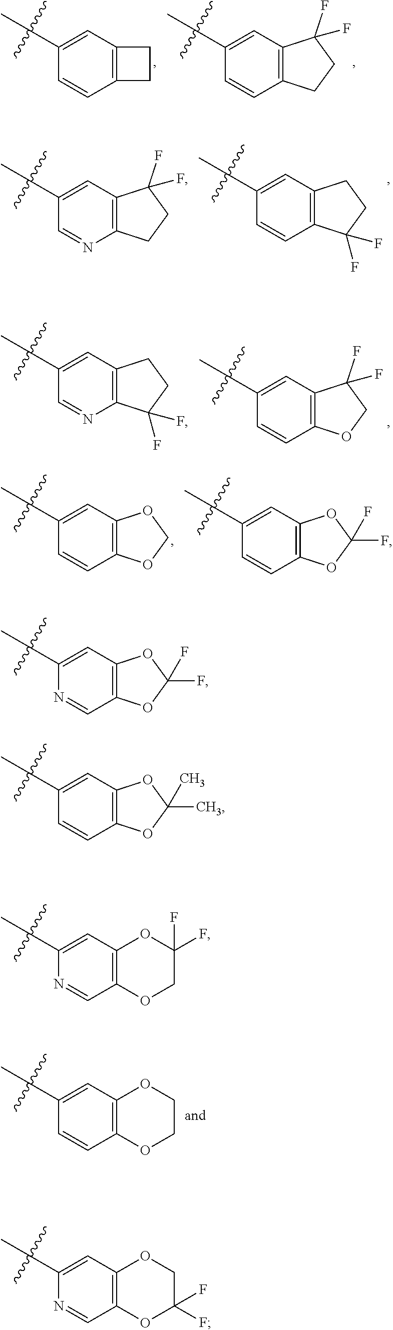

and

W, Z, R², m and n are as defined in formula (I).

In one embodiment of the disclosure, the compound of formula (I) is a compound of formula (IV-1):

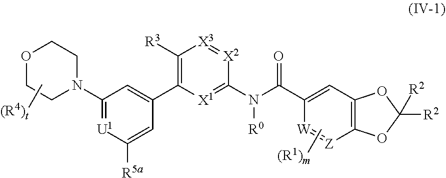

or a tautomer, racemate, enantiomer, diastereomer thereof, or mixture thereof, or a pharmaceutically acceptable salt, solvate or prodrug thereof, wherein:

R^{5a} is —O-L₂-OH; preferably R^{5a} is —O—(CH₂)₁₋₆—OH; more preferably R^{5a} is —O—(CH₂)₂—OH;

L₂ is alkylene; preferably L₂ is C₁₋₆ alkylene; more preferably L₂ is CH₂; and

W, Z, R⁰, X¹, X², X³, U¹, R¹ to R⁴, m and t are as defined in formula (I).

In one embodiment of the disclosure, the compound of formula (I) is a compound of formula (IV-2):

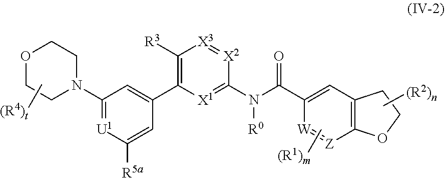

or a tautomer, racemate, enantiomer, diastereomer thereof, or mixture thereof, or a pharmaceutically acceptable salt, solvate or prodrug thereof, wherein:

R^{5a} is —O-L₂-OH; preferably R^{5a} is —O—(CH₂)₁₋₆—OH; more preferably R^{5a} is —O—(CH₂)₂—OH;

L₂ is alkylene; preferably L₂ is C₁₋₆ alkylene; more preferably L₂ is CH₂; and

W, Z, R⁰, X¹, X², X³, U¹, R¹ to R⁴, m, n and t are as defined in formula (I).

In one embodiment of the disclosure, the compound of formula (I) is a compound of formula (IV-3):

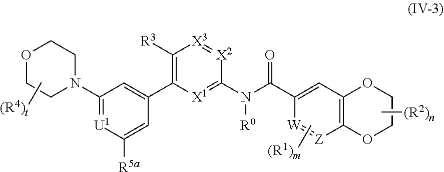

or a tautomer, racemate, enantiomer, diastereomer thereof, or mixture thereof, or a pharmaceutically acceptable salt, solvate or prodrug thereof, wherein:

R^{5a} is —O-L₂-OH; preferably R^{5a} is —O—(CH₂)₁₋₆—OH; more preferably R^{5a} is —O—(CH₂)₂—OH;

L₂ is alkylene; preferably L₂ is C₁₋₆ alkylene; more preferably L₂ is CH₂; and

W, Z, R⁰, X¹, X², X³, U¹, R¹ to R⁴, m, n and t are as defined in formula (I).

In one embodiment of the disclosure, the compound of formula (I) is selected from a compound of formula (IV-4):

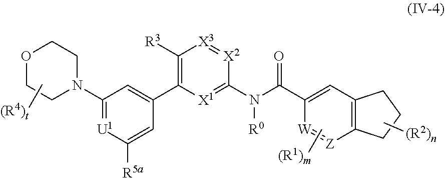

or a tautomer, racemate, enantiomer, diastereomer thereof, or mixture thereof, or a pharmaceutically acceptable salt, solvate or prodrug thereof, wherein:

R^{5a} is —O-L₂-OH; preferably R^{5a} is —O—(CH₂)₁₋₆—OH; more preferably R^{5a} is —O—(CH₂)₂—OH;

L₂ is alkylene; preferably L₂ is C₁₋₆ alkylene; more preferably L₂ is CH₂; and

W, Z, R⁰, X¹, X², X³, U¹, R¹ to R⁴, m, n and t are as defined in formula (I).

In one embodiment, the disclosure provides a compound of formula (I), or a tautomer, racemate, enantiomer, diastereomer thereof, or mixture thereof, or a pharmaceutically acceptable salt, solvate or prodrug thereof, wherein R⁰ is hydrogen or alkyl; preferably R⁰ is hydrogen or C₁₋₆ alkyl.

In one embodiment, the disclosure provides a compound of formula (I), or a tautomer, racemate, enantiomer, diastereomer thereof, or mixture thereof, or a pharmaceutically acceptable salt, solvate or prodrug thereof, wherein R¹ at each occurrence is selected from hydrogen, halogen and alkyl; preferably R¹ is selected from hydrogen, halogen and C₁₋₆ alkyl.

In one embodiment, the disclosure provides a compound of formula (I), or a tautomer, racemate, enantiomer, diastereomer thereof, or mixture thereof, or a pharmaceutically acceptable salt, solvate or prodrug thereof, wherein R² at each occurrence is selected from hydrogen, hydroxyl, halogen, alkyl, alkenyl, alkynyl, haloalkyl and hydroxyalkyl; preferably R² is selected from hydrogen, halogen, C₁₋₆ alkyl and C₁₋₆ haloalkyl; more preferably R² is selected from hydrogen, fluoro, methyl and trifluoromethyl.

In one embodiment, the disclosure provides a compound of formula (I), or a tautomer, racemate, enantiomer, diastereomer thereof, or mixture thereof, or a pharmaceutically acceptable salt, solvate or prodrug thereof, wherein X¹ is CH, X² and X³ are identical or different, and each is independently selected from CH and N; provided that X² and X³ are not N at the same time.

In one embodiment, the disclosure provides a compound of formula (I), or a tautomer, racemate, enantiomer, diastereomer thereof, or mixture thereof, or a pharmaceutically acceptable salt, solvate or prodrug thereof, wherein U¹ is CR⁵ or N; R⁵ at each occurrence is selected from hydrogen, halogen, alkyl, alkoxy, hydroxyl, cyano, amino, —O-L₂-OH, cycloalkyl, heterocyclyl, aryl, heteroaryl, —O-cycloalkyl, —O-heterocyclyl, —O-aryl and —O-heteroaryl; wherein the alkyl, alkoxy, cycloalkyl, heterocyclyl, aryl and heteroaryl is each optionally substituted with one or more groups selected from oxo, hydroxyl, halogen, alkyl, haloalkyl, hydroxyalkyl, cyano, amino and alkoxy; preferably R⁵ is selected from hydrogen, halogen, alkyl, alkoxy and —O-L₂-OH; wherein the alkyl and alkoxy is each optionally substituted with one or more groups selected from hydroxyl, halogen, alkyl, haloalkyl, hydroxyalkyl, cyano, amino and alkoxy; wherein L₂ is alkylene.

In one embodiment, the disclosure provides a compound of formula (I), or a tautomer, racemate, enantiomer, diastereomer thereof, or mixture thereof, or a pharmaceutically acceptable salt, solvate or prodrug thereof, wherein U¹ is CR⁵ or N; R⁵ is —O-L₂-OH, L₂ is alkylene; preferably R^{5a} is —O—(CH₂)₁₋₆—OH; more preferably R^{5a} is —O—(CH₂)₂—OH.

In one embodiment, the disclosure provides a compound of formula (I), or a tautomer, racemate, enantiomer, diastereomer thereof, or mixture thereof, or a pharmaceutically acceptable salt, solvate or prodrug thereof, wherein R³ is selected from hydrogen, hydroxyl, halogen, alkyl, alkenyl, alkynyl, haloalkyl and hydroxyalkyl; preferably R³ is C₁₋₆ alkyl; more preferably R³ is methyl.

In one embodiment, the disclosure provides a compound of formula (I), or a tautomer, racemate, enantiomer, diastereomer thereof, or mixture thereof, or a pharmaceutically acceptable salt, solvate or prodrug thereof, wherein R⁴ is hydrogen.

In one embodiment, the disclosure provides a compound of formula (I), or a tautomer, racemate, enantiomer, or diastereomer thereof, or a pharmaceutically acceptable salt, solvate or prodrug thereof, wherein:

ring A is an optionally substituted C₄-C₆ cycloalkyl or 4- or 6-membered heterocyclyl fused to the adjacent aromatic ring, wherein the heterocyclyl comprises one or two oxygen atoms in the ring;

X¹ is CH, X² and X³ are identical or different, and each is independently selected from CH and N; provided that X² and X³ are not N at the same time;

U¹ is CR⁵ or N; R⁵ is selected from hydrogen, halogen, and alkyl;

U² is CR^{5a}, and R^{5a} is —O-L₂-OH; preferably R^{5a} is —O—(CH₂)₁₋₆—OH; more preferably R^{5′} is —O—(CH₂)₂—OH;

U³ and U⁴ are each CH;

Y is O;

R⁰ is hydrogen or alkyl;

R¹ at each occurrence is independently selected from hydrogen, halogen, and alkyl;

R² at each occurrence is independently selected from hydrogen, hydroxyl, halogen, alkyl, alkenyl, alkynyl, haloalkyl, and hydroxyalkyl;

R³ is selected from hydrogen, hydroxyl, halogen, alkyl, alkenyl, alkynyl, haloalkyl and hydroxyalkyl; and

R⁴ is hydrogen.

Exemplified compounds of the disclosure include, but are not limited to:

| Compound | | |
|---|---|---|
| No. | Compound structure and name | |
| 1 | 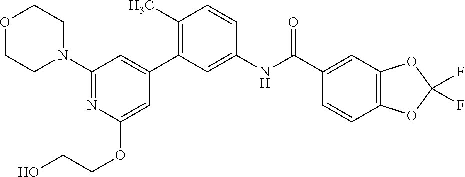 [SMILES: Cc1ccc(NC(=O)c2ccc3c(c2)OC(F)(F)O3)cc1-c1cc(OCCO)nc(N2CCOCC2)c1] | 1 |
| | 2,2-difluoro-N-(3-(2-(2-hydroxyethoxy)-6-morpholinopyridin-4-yl)- | |
| | 4-methylphenyl)benzo[d][1,3]dioxole-5-carboxamide 1 | |
| 2 | 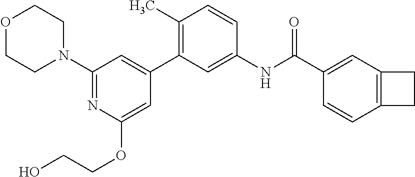 [SMILES: Cc1ccc(NC(=O)c2ccc3c(c2)CC3)cc1-c1cc(OCCO)nc(N2CCOCC2)c1] | 2 |
| | N-(3-(2-(2-hydroxyethoxy)-6-morpholinopyridin-4-yl)-4- | |
| | methylphenyl)bicyclo[4.2.0]octa-1(6),2,4-triene-3-carboxamide 2 | |
| 3 | 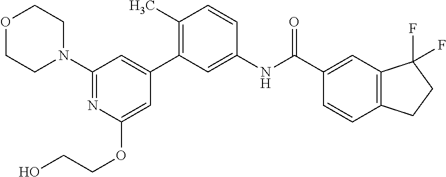 [SMILES: Cc1ccc(NC(=O)c2ccc3c(c2)C(F)(F)CC3)cc1-c1cc(OCCO)nc(N2CCOCC2)c1] | 3 |
| | 3,3-difluoro-N-(3-(2-(2-hydroxyethoxy)-6-morpholinopyridin-4-yl)- | |
| | 4-methylphenyl)-2,3-dihydro-1H-indene-5-carboxamide 3 | |
| 4 | 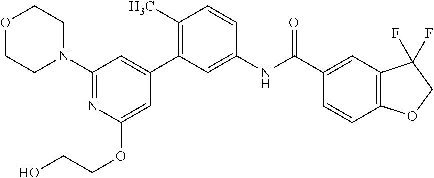 [SMILES: Cc1ccc(NC(=O)c2ccc3c(c2)C(F)(F)CO3)cc1-c1cc(OCCO)nc(N2CCOCC2)c1] | 4 |
| | 3,3-difluoro-N-(3-(2-(2-hydroxyethoxy)-6-morpholinopyridin-4-yl)- | |
| | 4-methylphenyl)-2,3-dihydrobenzofuran-5-carboxamide 4 | |
| 5 | 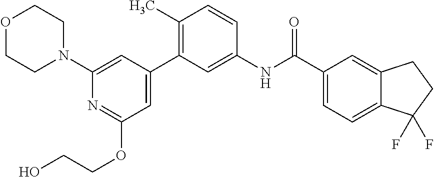 [SMILES: Cc1ccc(NC(=O)c2ccc3c(c2)CCC3(F)F)cc1-c1cc(OCCO)nc(N2CCOCC2)c1] | 5 |
| | 1,1-difluoro-N-(3-(2-(2-hydroxyethoxy)-6-morpholinopyridin-4-yl)- | |
| | 4-methylphenyl)-2,3-dihydro-1H-indene-5-carboxamide 5 | |
| 6 | 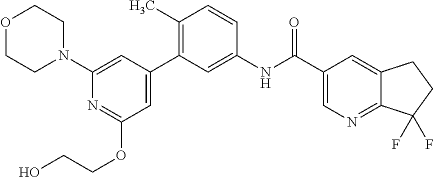 [SMILES: Cc1ccc(NC(=O)c2cnc3c(c2)CCC3(F)F)cc1-c1cc(OCCO)nc(N2CCOCC2)c1] | 6 |
| | 7,7-difluoro-N-(3-(2-(2-hydroxyethoxy)-6-morpholinopyridin-4-yl)- | |
| | 4-methylphenyl)-6,7-dihydro-5H-cyclopenta[b]pyridine-3- | |
| | carboxamide 6 | |
| 7 | 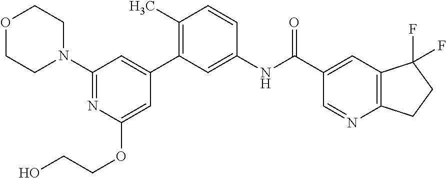 [SMILES: Cc1ccc(NC(=O)c2cnc3c(c2)C(F)(F)CC3)cc1-c1cc(OCCO)nc(N2CCOCC2)c1] | 7 |
| | 5,5-difluoro-N-(3-(2-(2-hydroxyethoxy)-6-morpholinopyridin-4-yl)- | |
| | 4-methylphenyl)-6,7-dihydro-5H-cyclopenta[b]pyridine-3- | |
| | carboxamide 7 | |
| 8 | 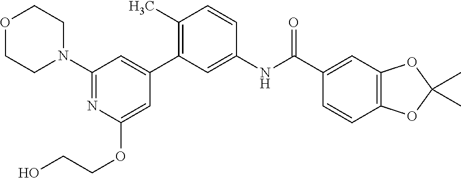 [SMILES: Cc1ccc(NC(=O)c2ccc3c(c2)OC(C)(C)O3)cc1-c1cc(OCCO)nc(N2CCOCC2)c1] | 8 |
| | N-(3-(2-(2-hydroxyethoxy)-6-morpholinopyridin-4-yl)-4- | |
| | methylphenyl)-2,2-dimethylbenzo[d][1,3]dioxole-5-carboxamide 8 | |
| 9 | 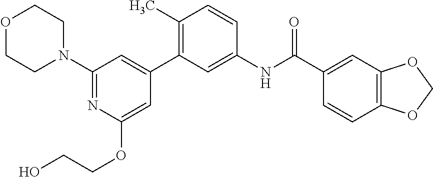 [SMILES: Cc1ccc(NC(=O)c2ccc3c(c2)OCO3)cc1-c1cc(OCCO)nc(N2CCOCC2)c1] | 9 |
| | N-(3-(2-(2-hydroxyethoxy)-6-morpholinopyridin-4-yl)-4- | |
| | methylphenyl)benzo[d][1,3]dioxole-5-carboxamide 9 | |
| 10 | 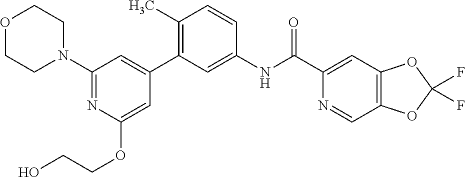 [SMILES: Cc1ccc(NC(=O)c2cc3c(cn2)OC(F)(F)O3)cc1-c1cc(OCCO)nc(N2CCOCC2)c1] | 10 |
| | 2,2-difluoro-N-(3-(2-(2-hydroxyethoxy)-6-morpholinopyridin-4-yl)- | |
| | 4-methylphenyl)-[1,3]dioxolo[4,5-c]pyridine-6-carboxamide 10 | |
| 11 | 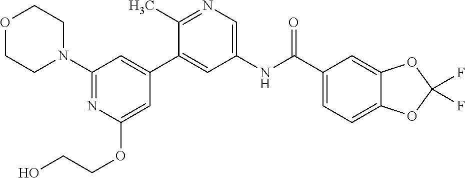 [SMILES: Cc1ncc(NC(=O)c2ccc3c(c2)OC(F)(F)O3)cc1-c1cc(OCCO)nc(N2CCOCC2)c1] | 11 |
| | 2,2-difluoro-N-(2′-(2-hydroxyethoxy)-2-methyl-6′-morpholino-[3,4′- | |
| | bipyridin]-5-yl)benzo[d][1,3]dioxole-5-carboxamide 11 | |
| 12 | 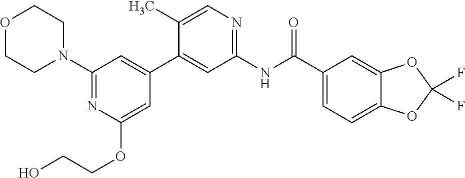 [SMILES: Cc1cnc(NC(=O)c2ccc3c(c2)OC(F)(F)O3)cc1-c1cc(OCCO)nc(N2CCOCC2)c1] | 12 |
| | 2,2-difluoro-N-(2′-(2-hydroxyethoxy)-5-methyl-6′-morpholino-[4,4′- | |
| | bipyridin]-2-yl)benzo[d][1,3]dioxole-5-carboxamide 12 | |
| 13 | 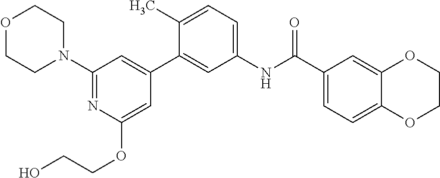 [SMILES: Cc1ccc(NC(=O)c2ccc3c(c2)OCCO3)cc1-c1cc(OCCO)nc(N2CCOCC2)c1] | |

N-(3-(2-(2-hydroxyethoxy)-6-morpholinopyridin-4-yl)-4-
methylphenyl)-2,3-dihydrobenzo[b][1,4]dioxine-6-carboxamide 13

In another aspect, this disclosure provides a compound of formula (IIIA):

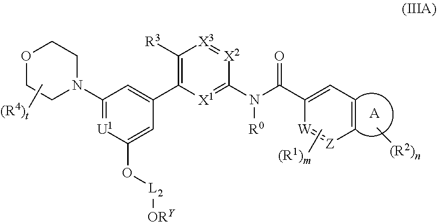

or a tautomer, racemate, enantiomer, diastereomer thereof, or mixture thereof, or a pharmaceutically acceptable salt, solvate or prodrug thereof, wherein:

L₂ is alkylene; preferably L₂ is C₁₋₆ alkylene; more preferably L₂ is CH₂;

R^{Y} is a hydroxyl protecting group; preferably R^{Y} is selected from tert-butyldimethylsilyl (TBS) and ethoxyethyl, 2-tetrahydropyranyl (THP); and

W, Z, R⁰, X¹, X², X³, U¹, ring A, R¹ to R⁴, m, n and t are as defined in formula (III).

In another aspect, this disclosure provides a compound of formula (IVA-1):

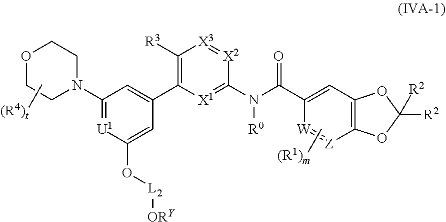

or a tautomer, racemate, enantiomer, diastereomer thereof, or mixture thereof, or a pharmaceutically acceptable salt, solvate or prodrug thereof, wherein:

L₂ is alkylene; preferably L₂ is C₁₋₆ alkylene; more preferably L₂ is CH₂;

R^{Y} is a hydroxyl protecting group; preferably R^{Y} is selected from TBS and THP; and

W, Z, R⁰, X¹, X², X³, U¹, R¹ to R⁴, m and t are as defined in formula (IV-1).

In another aspect, this disclosure provides a compound of formula (IVA-2):

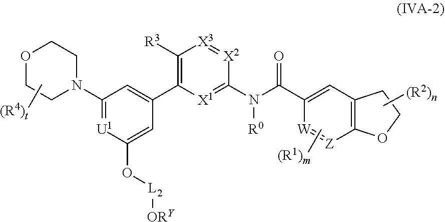

or a tautomer, racemate, enantiomer, diastereomer thereof, or mixture thereof, or a pharmaceutically acceptable salt, solvate or prodrug thereof, wherein:

L₂ is alkylene; preferably L₂ is C₁₋₆ alkylene; more preferably L₂ is CH₂;

R^{Y} is a hydroxyl protecting group; preferably R^{Y} is selected from TBS and THP; and

W, Z, R⁰, X¹, X², X³, U¹, R¹ to R⁴, m, n and t are as defined in formula (IV-2).

In another aspect, this disclosure provides a compound of formula (IVA-3):

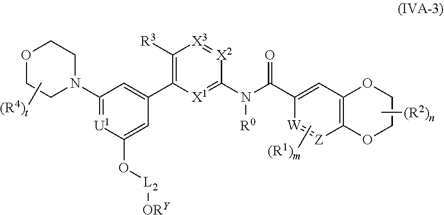

or a tautomer, racemate, enantiomer, diastereomer thereof, or mixture thereof, or a pharmaceutically acceptable salt, solvate or prodrug thereof, wherein:

L₂ is alkylene; preferably L₂ is C₁₋₆ alkylene; more preferably L₂ is CH₂;

R^{Y} is a hydroxyl protecting group; preferably R^{Y} is selected from TBS and THP; and

W, Z, R⁰, X¹, X², X³, U¹, R¹ to R⁴, m, n and t are as defined in formula (IV-3).

In another aspect, this disclosure provides a compound of formula (IVA-4):

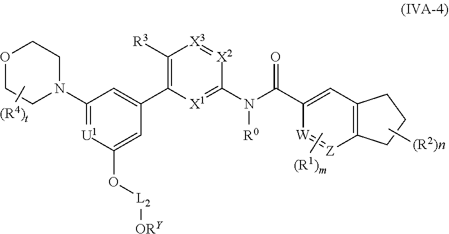

or a tautomer, racemate, enantiomer, diastereomer thereof, or mixture thereof, or a pharmaceutically acceptable salt, solvate or prodrug thereof, wherein:

L₂ is alkylene; preferably L₂ is C₁₋₆ alkylene; more preferably L₂ is CH₂;

R^{Y} is a hydroxyl protecting group; preferably R^{Y} is selected from TBS and THP; and

W, Z, R⁰, X¹, X², X³, U¹, R¹ to R⁴, m, n and t are as defined in formula (IV-4).

Exemplified compounds of the disclosure include, but are not limited to:

| Compound | | |
|---|---|---|
| No. | Compound structure and name | |
| 1a | 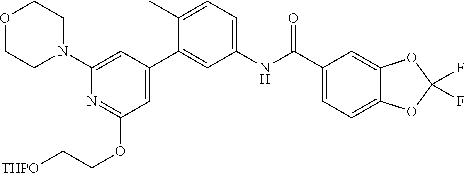 | 1a |
| | 2,2-difluoro-N-(4-methyl-3-(2-morpholino-6-(2-((tetrahydro-2H- | |
| | pyran-2-yl)oxy)ethoxy)pyridin-4-yl)phenyl)benzo[d][1,3]dioxole-5- | |
| | carboxamide 1a | |
| 2b | 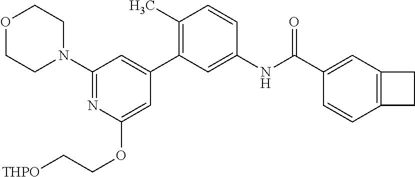 | 2b |
| | N-(4-methyl-3-(2-morpholino-6-(2-((tetrahydro-2H-pyran-2- | |
| | yl)oxy)ethoxy)pyridin-4-yl)phenyl)bicyclo[4.2.0]octa-1(6),2,4- | |
| | triene-3-carboxamide 2b | |
| 3g | 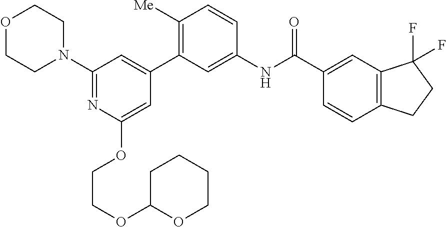 [SMILES: Cc1ccc(NC(=O)c2ccc3c(c2)C(F)(F)CC3)cc1-c1cc(OCCOC2CCCCO2)nc(N2CCOCC2)c1] | 3g |
| | 3,3-difluoro-N-(4-methyl-3-(2-morpholino-6-(2-((tetrahydro-2H- | |
| | pyran-2-yl)oxy)ethoxy)pyridin-4-yl)phenyl)-2,3-dihydro-1H-indene- | |
| | 5-carboxamide 3g | |
| 4d | 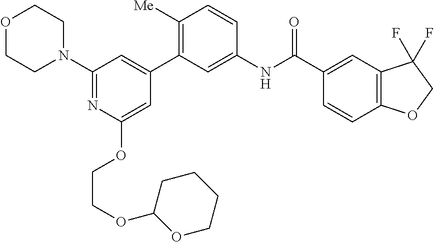 [SMILES: Cc1ccc(NC(=O)c2ccc3c(c2)C(F)(F)CO3)cc1-c1cc(OCCOC2CCCCO2)nc(N2CCOCC2)c1] | 4d |
| | 3,3-difluoro-N-(4-methyl-3-(2-morpholino-6-(2-((tetrahydro-2H- | |
| | pyran-2-yl)oxy)ethoxy)pyridin-4-yl)phenyl)-2,3-dihydrobenzofuran- | |
| | 5-carboxamide 4d | |
| 5g | 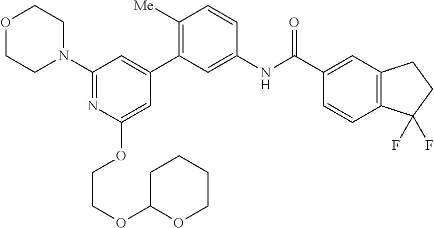 [SMILES: Cc1ccc(NC(=O)c2ccc3c(c2)CCC3(F)F)cc1-c1cc(OCCOC2CCCCO2)nc(N2CCOCC2)c1] | 5g |
| | 1,1-difluoro-N-(4-methyl-3-(2-morpholino-6-(2-((tetrahydro-2H- | |
| | pyran-2-yl)oxy)ethoxy)pyridin-4-yl)phenyl)-2,3-dihydro-1H-indene- | |
| | 5-carboxamide 5g | |
| 6f | 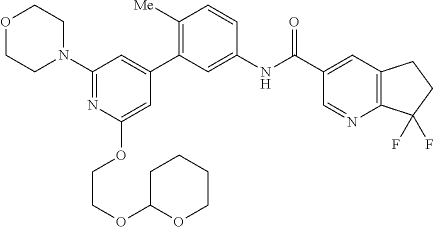 [SMILES: Cc1ccc(NC(=O)c2cnc3c(c2)CCC3(F)F)cc1-c1cc(OCCOC2CCCCO2)nc(N2CCOCC2)c1] | 6f |
| | 7,7-difluoro-N-(4-methyl-3-(2-morpholino-6-(2-((tetrahydro-2H- | |
| | pyran-2-yl)oxy)ethoxy)pyridin-4-yl)phenyl)-6,7-dihydro-5H- | |
| | cyclopenta[b]pyridine-3-carboxamide 6f | |
| 7f | 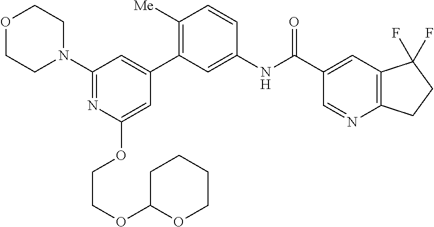 [SMILES: Cc1ccc(NC(=O)c2cnc3c(c2)C(F)(F)CC3)cc1-c1cc(OCCOC2CCCCO2)nc(N2CCOCC2)c1] | 7f |
| | 5,5-difluoro-N-(4-methyl-3-(2-morpholino-6-(2-((tetrahydro-2H- | |
| | pyran-2-yl)oxy)ethoxy)pyridin-4-yl)phenyl)-6,7-dihydro-5H- | |
| | cyclopenta[b]pyridine-3-carboxamide 7f | |
| 8e | 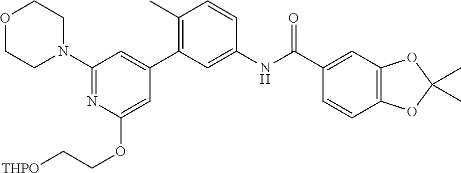 | 8e |
| | 2,2-dimethyl-N-(4-methyl-3-(2-morpholino-6-(2-((tetrahydro-2H- | |
| | pyran-2-yl)oxy)ethoxy)pyridin-4-yl)phenyl)benzo[d][1,3]dioxole-5- | |
| | carboxamide 8e | |
| 9b | 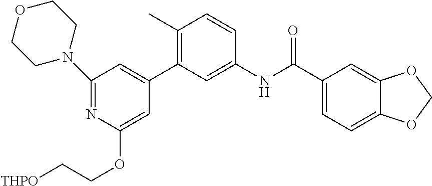 | 9b |
| | N-(4-methyl-3-(2-morpholino-6-(2-((tetrahydro-2H-pyran-2- | |
| | yl)oxy)ethoxy)pyridin-4-yl)phenyl)benzo[d][1,3]dioxole-5- | |
| | carboxamide 9b | |
| 10h | 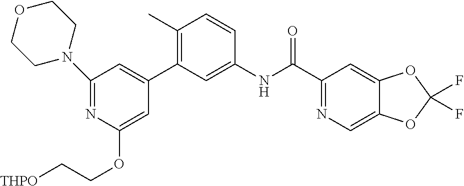 | 10h |
| | 2,2-difluoro-N-(4-methyl-3-(2-morpholino-6-(2-((tetrahydro-2H- | |
| | pyran-2-yl)oxy)ethoxy)pyridin-4-yl)phenyl)-[1,3]dioxolo[4,5- | |
| | c]pyridine-6-carboxamide 10h | |
| 11e | 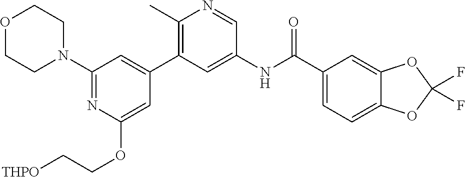 | 11e |
| | 2,2-difluoro-N-(2-methyl-2′-morpholino-6′-(2-((tetrahydro-2H- | |
| | pyran-2-yl)oxy)ethoxy)-[3,4′-bipyridin]-5-yl)benzo[d][1,3]dioxole- | |
| | 5-carboxamide 11e | |
| 12e | 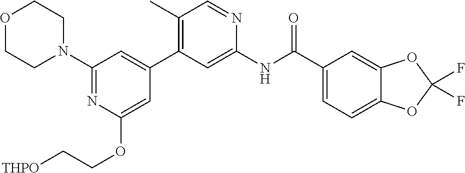 | 12e |
| | 2,2-difluoro-N-(5-methyl-2′-morpholino-6′-(2-((tetrahydro-2H- | |
| | pyran-2-yl)oxy)ethoxy)-[4,4′-bipyridin]-2-yl)benzo[d][1,3]dioxole- | |
| | 5-carboxamide 12e | |
| 13a | 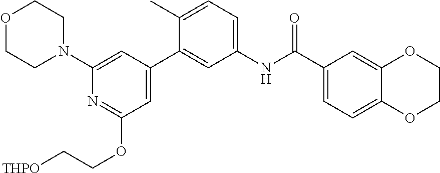 | 13a |

N-(4-methyl-3-(2-morpholino-6-(2-((tetrahydro-2H-pyran-2-
yl)oxy)ethoxy)pyridin-4-yl)phenyl)-2,3-
dihydrobenzo[b][1,4]dioxine-6-carboxamide 13a

As a person of ordinary skill in the art would understand, any and all reasonable combinations of the embodiments disclosed herein, especially with regard to the definitions of any substituents, e.g., W, Z, Y, L₁, X¹, X², X³, U¹, U², u³, U⁴ ring A, R¹ to R⁴, R⁰, m, n and t, or the like, in the compounds of formulae (I) to (IV-4), (IIIA), and (IVA-1) to (IVA-4), or the like, are all encompassed by the present invention.

In another aspect, this disclosure provides a process of preparing the compound of formula (III), or a tautomer, racemate, enantiomer, diastereomer thereof, or mixture thereof, or a pharmaceutically acceptable salt, solvate or prodrug thereof, comprising a step of:

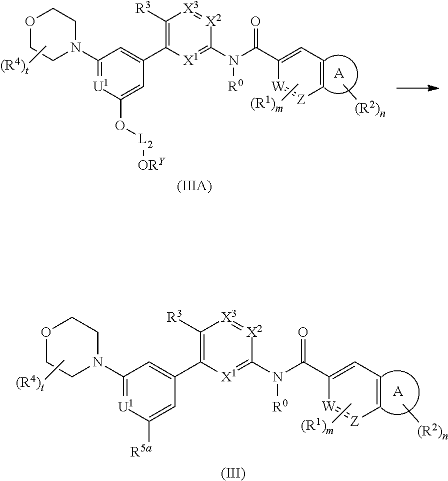

removing the hydroxyl protecting group of Formula (IIIA) or a tautomer, racemate, enantiomer, diastereomer thereof, or mixture thereof, or a pharmaceutically acceptable salt, solvate or prodrug thereof, to obtain the compound of formula (III) or a tautomer, racemate, enantiomer, diastereomer thereof, or mixture thereof, or a pharmaceutically acceptable salt, solvate or prodrug thereof, wherein:

L₂ is alkylene; preferably L₂ is C₁₋₆ alkylene; more preferably L₂ is CH₂;

R^{Y} is a hydroxyl protecting group; preferably R^{Y} is selected from TBS and THP;

R^{5a} is —O-L₂-OH; preferably R^{5a} is —O—(CH₂)₁₋₆—OH; more preferably R^{5a} is —O—(CH₂)₂—OH; and

W, Z, R⁰, X¹, X², X³, U¹, ring A, R¹ to R⁴, m, n and t are as defined in formula (III).

In another aspect, this disclosure provides a process of preparing the compound of formula (III), or a tautomer, racemate, enantiomer, diastereomer thereof, or mixture thereof, or a pharmaceutically acceptable salt, solvate or prodrug thereof, comprising a step of:

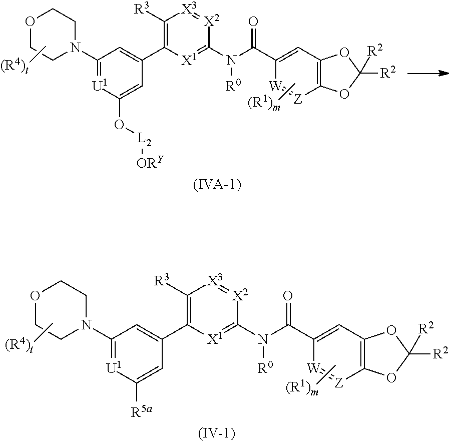

removing the hydroxyl protecting group of Formula (IVA-1) or a tautomer, racemate, enantiomer, diastereomer thereof, or mixture thereof, or a pharmaceutically acceptable salt, solvate or prodrug thereof, to obtain the compound of formula (IV-1) or a tautomer, racemate, enantiomer, diastereomer thereof, or mixture thereof, or a pharmaceutically acceptable salt, solvate or prodrug thereof, wherein:

L₂ is alkylene; preferably L₂ is C₁₋₆ alkylene; more preferably L₂ is CH₂;

R^{Y} is a hydroxyl protecting group; preferably R^{Y} is selected from TBS and THP;

R^{5a} is —O-L₂-OH; preferably R^{5a} is —O—(CH₂)₁₋₆—OH; more preferably R^{5a} is —O—(CH₂)₂—OH; and

W, Z, R⁰, X¹, X², X³, U¹, R¹ to R⁴, m and t are as defined in formula (IV-1).

In another aspect, this disclosure provides a process of preparing the compound of formula (IV-2), or a tautomer, racemate, enantiomer, diastereomer thereof, or mixture thereof, or a pharmaceutically acceptable salt, solvate or prodrug thereof, comprising a step of:

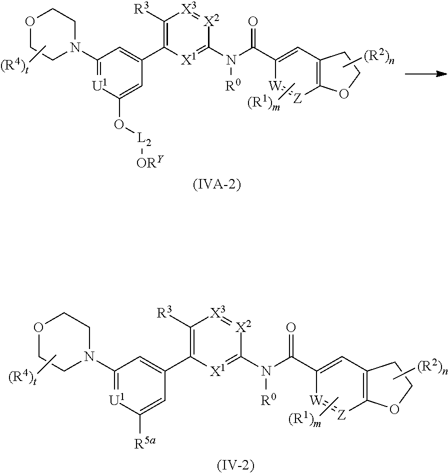

removing the hydroxyl protecting group of Formula (IVA-2) or a tautomer, racemate, enantiomer, diastereomer thereof, or mixture thereof, or a pharmaceutically acceptable salt, solvate or prodrug thereof, to obtain the compound of formula (IV-2) or a tautomer, racemate, enantiomer, diastereomer thereof, or mixture thereof, or a pharmaceutically acceptable salt, solvate or prodrug thereof, wherein:

L₂ is alkylene; preferably L₂ is C₁₋₆ alkylene; more preferably L₂ is CH₂;

R^{Y} is a hydroxyl protecting group; preferably R^{Y} is selected from TBS and THP;

R^{5a} is —O-L₂-OH; preferably R^{5a} is —O—(CH₂)₁₋₆—OH; more preferably R^{5a} is —O—(CH₂)₂—OH; and

W, Z, R⁰, X¹, X², X³, U¹, R¹ to R⁴, m, n and t are as defined in formula (IV-2).

In another aspect, this disclosure provides a process of preparing the compound of formula (IV-3), or a tautomer, racemate, enantiomer, diastereomer thereof, or mixture thereof, or a pharmaceutically acceptable salt, solvate or prodrug thereof, comprising a step of:

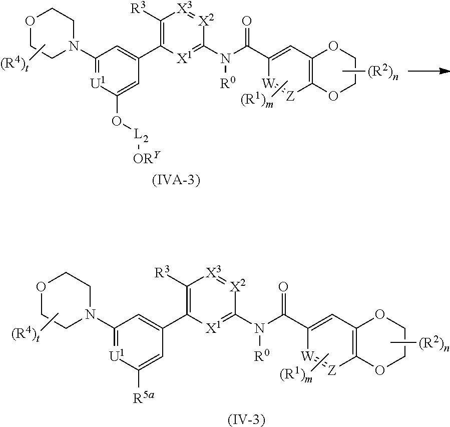

removing the hydroxyl protecting group of Formula (IVA-3) or a tautomer, racemate, enantiomer, diastereomer thereof, or mixture thereof, or a pharmaceutically acceptable salt, solvate or prodrug thereof, to obtain the compound of formula (IV-3) or a tautomer, racemate, enantiomer, diastereomer thereof, or mixture thereof, or a pharmaceutically acceptable salt, solvate or prodrug thereof, wherein:

L₂ is alkylene; preferably L₂ is C₁₋₆ alkylene; more preferably L₂ is CH₂;

R^{Y} is a hydroxyl protecting group; preferably R^{Y} is selected from TBS and THP;

R^{5a} is —O-L₂-OH; preferably R^{5a} is —O—(CH₂)₁₋₆—OH; more preferably R^{5a} is —O—(CH₂)₂—OH; and

W, Z, R⁰, X¹, X², X³, U¹, R¹ to R⁴, m, n and t are as defined in formula (IV-3).

In another aspect, this disclosure provides a process of preparing the compound of formula (IV-4), or a tautomer, racemate, enantiomer, diastereomer thereof, or mixture thereof, or a pharmaceutically acceptable salt, solvate or prodrug thereof, comprising a step of:

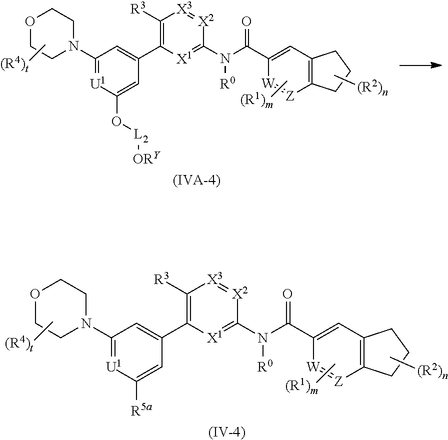

removing the hydroxyl protecting group of Formula (IVA-4) or a tautomer, racemate, enantiomer, diastereomer thereof, or mixture thereof, or a pharmaceutically acceptable salt, solvate or prodrug thereof, to obtain the compound of formula (IV-4) or a tautomer, racemate, enantiomer, diastereomer thereof, or mixture thereof, or a pharmaceutically acceptable salt, solvate or prodrug thereof, wherein:

L₂ is alkylene; preferably L₂ is C₁₋₆ alkylene; more preferably L₂ is CH₂;

R^{Y} is a hydroxyl protecting group; preferably R^{Y} is selected from TBS and THP;

R^{5a} is —O-L₂-OH; preferably R^{5a} is —O—(CH₂)₁₋₆—OH; more preferably R^{5a} is —O—(CH₂)₂—OH; and

W, Z, R⁰, X¹, X², X³, U¹, R¹ to R⁴, m, n and t are as defined in formula (IV-4).

The present disclosure also provides a pharmaceutical composition, comprising a compound of formula (I), or a tautomer, racemate, enantiomer, diastereomer thereof, or mixture thereof, or a pharmaceutically acceptable salt, solvate or prodrug thereof, and one or more pharmaceutically acceptable carriers, diluents and/or other excipients.

The present disclosure also provides a method of inhibiting RAF, comprising a step of administering to a subject in need thereof a therapeutically effective amount of a compound of formula (I), or a tautomer, racemate, enantiomer, diastereomer thereof, or mixture thereof, or a pharmaceutically acceptable salt, solvate or prodrug thereof, or a pharmaceutical composition thereof.

The present disclosure also provides a method of treating a RAF-mediated disease or disorder, comprising a step of administering to a subject in need thereof a therapeutically effective amount of a compound of formula (I), or a tautomer, racemate, enantiomer, diastereomer thereof, or mixture thereof, or a pharmaceutically acceptable salt, solvate or prodrug thereof, or a pharmaceutical composition thereof.

In some embodiments, the RAF-mediated disease or disorder is a cancer.

In some embodiments, the cancer is selected from lymphoma, leukemia, breast cancer, lung cancer, prostate cancer, ovarian cancer, liver cancer, melanoma, rhabdomyosarcoma, synovial sarcoma, mesothelioma, cervical cancer, colon cancer, rectal cancer, stomach cancer, pancreatic cancer, brain cancer, skin cancer, oral cancer, bone cancer, kidney cancer, bladder cancer, fallopian tube tumor, ovarian tumor, peritoneal tumor, glioma, glioblastoma, head and neck cancer, and myeloma; preferably lymphoma, leukemia, breast cancer, lung cancer, prostate cancer, ovarian cancer, liver cancer, melanoma, rhabdomyosarcoma, synovial sarcoma and mesothelioma.

In another aspect, the present disclosure also relates to use of a compound of formula (I), or a tautomer, mesomer, racemate, enantiomer, diastereomer, or mixture thereof, or a pharmaceutically acceptable salt, solvate or prodrug thereof, or a pharmaceutical composition thereof, in the preparation of a medicament for inhibition of RAF.

In another aspect, the present disclosure also relates to use of a compound of formula (I) or a tautomer, mesomer, racemate, enantiomer, diastereomer, or mixture thereof, or a pharmaceutically acceptable salt thereof, solvate or prodrug thereof, or a pharmaceutical composition thereof, in the preparation of a medicament for treating RAF mediated disease or disorder; preferably, wherein the RAF-mediated disease or disorder is cancer.

In some embodiments, the cancer is selected from lymphoma, leukemia, breast cancer, lung cancer, prostate cancer, ovarian cancer, liver cancer, melanoma, rhabdomyosarcoma, synovial sarcoma, mesothelioma, cervical cancer, colon cancer, rectal cancer, stomach cancer, pancreatic cancer, brain cancer, skin cancer, oral cancer, bone cancer, kidney cancer, bladder cancer, fallopian tube tumor, ovarian tumor, peritoneal tumor, glioma, glioblastoma, head and neck cancer, and myeloma; preferably lymphoma, leukemia, breast cancer, lung cancer, prostate cancer, ovarian cancer, liver cancer, melanoma, rhabdomyosarcoma, synovial sarcoma and mesothelioma.

The present disclosure further relates to the compound of formula (I), or a tautomer, mesomer, racemate, enantiomer, diastereomer, or mixture thereof, or a pharmaceutically acceptable salt thereof, solvate or prodrug thereof, or a pharmaceutical composition thereof, for use as a medicament.

The present disclosure also relates to the compound of formula (I), or a tautomer, mesomer, racemate, enantiomer, diastereomer, or mixture thereof, or a pharmaceutically acceptable salt thereof, solvate or prodrug thereof, or a pharmaceutical composition thereof, for use in inhibiting RAF.

The present disclosure also relates to a combination of a compound of formula (I), or a tautomer, mesomer, racemate, enantiomer, diastereomer, or mixture thereof, or a pharmaceutically acceptable salt, solvate or prodrug thereof, or a pharmaceutical composition thereof, with a second agent for use in treating a RAF-mediated disease or disorder; preferably, wherein the RAF-mediated disease or disorder is cancer. The cancer is selected from lymphoma, leukemia, breast cancer, lung cancer, prostate cancer, ovarian cancer, liver cancer, melanoma, rhabdomyosarcoma, synovial sarcoma, mesothelioma, cervical cancer, colon cancer, rectal cancer, stomach cancer, pancreatic cancer, brain cancer, skin cancer, oral cancer, bone cancer, kidney cancer, bladder cancer, fallopian tube tumor, ovarian tumor, peritoneal tumor, glioma, glioblastoma, head and neck cancer, and myeloma; preferably lymphoma, leukemia, breast cancer, lung cancer, prostate cancer, ovarian cancer, liver cancer, melanoma, rhabdomyosarcoma, synovial sarcoma and mesothelioma.

The compositions of this disclosure can be formulated by conventional methods using one or more pharmaceutically acceptable carriers. Thus, the active compounds of this disclosure can be formulated as various dosage forms for oral, buccal, intranasal, parenteral (e.g., intravenous, intramuscular or subcutaneous), rectal administration, inhalation or insufflation administration. The compounds of this disclosure can also be formulated as sustained release dosage forms.

Common formulations include a tablet, troche, lozenge, aqueous or oily suspension, dispersible powder or granule, emulsion, hard or soft capsule, or syrup or elixir. Oral compositions can be prepared according to any known method in the art for the preparation of pharmaceutical compositions. Such compositions can contain one or more additives selected from sweeteners, flavoring agents, colorants and preservatives, in order to provide a pleasing and palatable pharmaceutical preparation. Tablets contain the active ingredient and nontoxic pharmaceutically acceptable excipients suitable for the manufacture of tablets. These excipients can be inert excipients, granulating agents, disintegrating agents, and lubricants. The tablet can be uncoated or coated by means of a known technique to mask the taste of the drug or delay the disintegration and absorption of the drug in the gastrointestinal tract, thereby providing sustained release over an extended period. For example, water soluble taste masking materials can be used.

Oral formulations can also be provided as soft gelatin capsules in which the active ingredient is mixed with an inert solid diluent, or the active ingredient is mixed with a water soluble carrier.

An aqueous suspension contains the active ingredient in admixture with excipients suitable for the manufacture of an aqueous suspension. Such excipients are suspending agents, dispersants or humectants, and can be naturally occurring phospholipids. The aqueous suspension can also contain one or more preservatives, one or more colorants, one or more flavoring agents, and one or more sweeteners.

An oil suspension can be formulated by suspending the active ingredient in a vegetable oil, or in a mineral oil. The oil suspension can contain a thickener. The aforementioned sweeteners and flavoring agents can be added to provide a palatable preparation. These compositions can be preserved by adding an antioxidant.

The active ingredient and the dispersants or wetting agents, suspending agent or one or more preservatives can be prepared as a dispersible powder or granule suitable for the preparation of an aqueous suspension by adding water. Suitable dispersants or wetting agents and suspending agents are exemplified by those already mentioned above. Additional excipients, such as sweeteners, flavoring agents and colorants, can also be added. These compositions can be preserved by adding an antioxidant such as ascorbic acid.

The present pharmaceutical composition can also be in the form of an oil-in-water emulsion. The oil phase can be a vegetable oil, or a mineral oil, or mixture thereof. Suitable emulsifying agents can be naturally occurring phospholipids. Sweeteners can be used. Such formulations can also contain moderators, preservatives, colorants and antioxidants.

The pharmaceutical composition can be in the form of a sterile injectable aqueous solution. The acceptable vehicles and solvents that can be employed are water, Ringer's solution and isotonic sodium chloride solution. The sterile injectable preparation can also be a sterile injectable oil-in-water microemulsion in which the active ingredient is dissolved in the oil phase. The injectable solution or microemulsion can be introduced into an individual's bloodstream by local bolus injection. Alternatively, it can be advantageous to administer the solution or microemulsion in such a way as to maintain a constant circulating concentration of the present compound. In order to maintain such a constant concentration, a continuous intravenous delivery device can be utilized. An example of such a device is Deltec CADD-PLUS™ 5400 intravenous injection pump.

The pharmaceutical composition can be in the form of a sterile injectable aqueous or oily suspension for intramuscular and subcutaneous administration. Such a suspension can be formulated with suitable dispersants or wetting agents and suspending agents as described above according to known techniques. The sterile injectable preparation can also be a sterile injectable solution or suspension prepared in a nontoxic parenterally acceptable diluent or solvent. Moreover, sterile fixed oils can easily be used as a solvent or suspending medium, and fatty acids can also be used to prepare injections.

The present compound can be administered in the form of a suppository for rectal administration. These pharmaceutical compositions can be prepared by mixing the drug with a suitable non-irritating excipient that is solid at ordinary temperatures, but liquid in the rectum, thereby melting in the rectum to release the drug.

For buccal administration, the compositions can be formulated as tablets or lozenges by conventional means.

For intranasal administration or administration by inhalation, the active compounds of the present disclosure are conveniently delivered in the form of a solution or suspension released from a pump spray container that is squeezed or pumped by the patient, or as an aerosol spray released from a pressurized container or nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. The pressurized container or nebulizer can contain a solution or suspension of the active compound. Capsules or cartridges (for example, made from gelatin) for use in an inhaler or insufflator can be formulated containing a powder mix of the present disclosure and a suitable powder base such as lactose or starch.

It is well known to those skilled in the art that the dosage of a drug depends on a variety of factors, including but not limited to, the following factors: activity of the specific compound, age, weight, general health, behavior, diet of the patient, administration time, administration route, excretion rate, drug combination and the like. In addition, the best treatment, such as treatment mode, daily dose of the compound of formula (I) or the type of pharmaceutically acceptable salt thereof can be verified by traditional therapeutic regimens.

Unless otherwise stated, the terms used in the specification and claims have the meanings described below.

“Alkyl” refers to a saturated aliphatic hydrocarbon group including C₁-C₁₂ straight chain and branched chain groups. In some embodiments, sometimes preferably, an alkyl group is an alkyl having 1 to 8 carbon atom(s) (such as 1, 2, 3, 4, 5, 6, 7 and 8 carbon atom(s)). Representative examples include, but are not limited to methyl, ethyl, n-propyl, isopropyl, n-butyl, isobutyl, tert-butyl, sec-butyl, n-pentyl, 1,1-dimethyl propyl, 1,2-dimethyl propyl, 2,2-dimethyl propyl, 1-ethyl propyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl, n-heptyl, 2-methylhexyl, 3-methylhexyl, 4-methylhexyl, 5-methylhexyl, 2,3-dimethylpentyl, 2,4-dimethylpentyl, 2,2-dimethylpentyl, 3,3-dimethylpentyl, 2-ethylpentyl, 3-ethylpentyl, n-octyl, 2,3-dimethylhexyl, 2,4-dimethylhexyl, 2,5-dimethylhexyl, 2,2-dimethylhexyl, 3,3-dimethylhexyl, 4,4-dimethylhexyl, 2-ethylhexyl, 3-ethylhexyl, 4-ethylhexyl, 2-methyl-2-ethylpentyl, 2-methyl-3-ethylpentyl, n-nonyl, 2-methyl-2-ethylhexyl, 2-methyl-3-ethylhexyl, 2,2-diethylpentyl, n-decyl, 3,3-diethylhexyl, 2,2-diethylhexyl, and the isomers of branched chain thereof. In some embodiments, sometimes more preferably an alkyl group is a lower alkyl having 1 to 6 carbon atom(s), and sometimes more preferably 1 to 4 carbon atom(s). Representative examples include, but are not limited to, methyl, ethyl, n-propyl, isopropyl, n-butyl, isobutyl, tert-butyl, sec-butyl, n-pentyl, 1,1-dimethylpropyl, 1,2-dimethylpropyl, 2,2-dimethylpropyl, 1-ethylpropyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl, etc. The alkyl group can be substituted or unsubstituted. When substituted, the substituent group(s) can be substituted at any available connection point, preferably the substituent group(s) is one or more, sometimes preferably 1 to 5, and sometimes more preferably 1 to 3, group(s) independently selected from alkyl, halogen, alkoxy, alkenyl, alkynyl, alkylthio, alkylamino, thiol, hydroxy, nitro, cyano, amino, cycloalkyl, heterocyclyl, aryl, heteroaryl, cycloalkoxy, cycloalkylthio, heterocyclylthio and oxo group.

“Alkenyl” refers to an alkyl defined as above that has at least two carbon atoms and at least one carbon-carbon double bond, for example, vinyl, 1-propenyl, 2-propenyl, 1-, 2-, or 3-butenyl, etc. preferably C₂₋₁₂ alkenyl, more preferably C₂₋₈ alkenyl, and sometimes more preferably C₂₋₆ alkenyl, and sometimes even more preferably C₂₋₄ alkenyl. The alkenyl group can be substituted or unsubstituted. When substituted, the substituent group(s) is preferably one or more, sometimes preferably 1 to 5, and sometimes more preferably 1 to 3, group(s) independently selected from alkyl, halogen, alkoxy, alkenyl, alkynyl, alkylthio, alkylamino, thiol, hydroxy, nitro, cyano, amino, cycloalkyl, heterocyclyl, aryl, heteroaryl, cycloalkoxy, cycloalkylthio, heterocyclylthio and oxo group.

“Alkynyl” refers to an alkyl defined as above that has at least two carbon atoms and at least one carbon-carbon triple bond, for example, ethynyl, 1-propynyl, 2-propynyl, 1-, 2-, or 3-butynyl etc., preferably C₂₋₁₂ alkynyl, sometimes more preferably C₂₋₈ alkynyl, sometimes more preferably C₂₋₆ alkynyl, and sometimes even more preferably C₂₋₄ alkynyl. The alkynyl group can be substituted or unsubstituted. When substituted, the substituent group(s) is preferably one or more, preferably 1 to 5, and sometimes more preferably 1 to 3, group(s) independently selected from alkyl, alkenyl, alkynyl, alkoxy, alkylthio, alkylamino, halogen, thiol, hydroxy, nitro, cyano, cycloalkyl, heterocyclyl, aryl, heteroaryl, cycloalkoxy, heterocyclyloxy, cycloalkylthio, heterocyclylthio and oxo group.

“Alkylene” refers to a saturated linear or branched divalent aliphatic hydrocarbon group, derived by removing two hydrogen atoms from the same carbon atom or two different carbon atoms of the parent alkane. The straight or branched chain group containing 1 to 12 carbon atom(s) (such as 1, 2, 3, 4, 5, 6, 7, 8, 9, 10, 11 and 12 carbon atom(s)), preferably has 1 to 8 carbon atom(s), more preferably 1 to 6 carbon atom(s), and sometimes more preferably 1 to 4 carbon atom(s). Non-limiting examples of alkylene groups include, but are not limited to, methylene (—CH₂—), 1,1-ethylene (—CH(CH₃)—), 1,2-ethylene (—CH₂CH₂)—, 1,1-propylene (—CH(CH₂CH₃)—), 1,2-propylene (—CH₂CH(CH₃)—), 1,3-propylene (—CH₂CH₂CH₂—), 1,4-butylidene (—CH₂CH₂CH₂CH₂—) etc. The alkylene group can be substituted or unsubstituted. When substituted, the substituent group(s) is preferably one or more, sometimes preferably 1 to 5, and sometimes more preferably 1 to 3, group(s) independently selected from selected from alkyl, alkenyl, alkynyl, alkoxy, alkylthio, alkylamino, halogen, thiol, hydroxy, nitro, cyano, cycloalkyl, heterocyclyl, aryl, heteroaryl, cycloalkoxy, heterocyclyloxy, cycloalkylthio, heterocyclylthio and oxo group.

“Alkenylene” refers to an alkylene defined as above that has at least two carbon atoms and at least one carbon-carbon double bond, preferably C₂₋₁₂ alkenylene, more preferably C₂₋₈ alkenylene, sometimes more preferably C₂₋₆ alkenylene, and sometimes even more preferably C₂₋₄ alkenylene. Non-limiting examples of alkenylene groups include, but are not limited to, —CH═CH—, —CH═CHCH₂—, —CH═CHCH₂CH₂—, —CH₂CH═CHCH₂— etc. The alkenylene group can be substituted or unsubstituted. When substituted, the substituent group(s) is preferably one or more, sometimes preferably 1 to 5, and sometimes more preferably 1 to 3, group(s) independently selected from selected from alkyl, alkenyl, alkynyl, alkoxy, alkylthio, alkylamino, halogen, thiol, hydroxy, nitro, cyano, cycloalkyl, heterocyclyl, aryl, heteroaryl, cycloalkoxy, heterocyclyloxy, cycloalkylthio, heterocyclylthio and oxo group.

“Cycloalkyl” refers to a saturated and/or partially unsaturated monocyclic or polycyclic hydrocarbon group having 3 to 12 carbon atoms (such as 3, 4, 5, 6, 7, 8, 9, 10, 11 and 12 carbon atoms), sometimes more preferably 3 to 8 carbon atoms, and sometimes even more preferably 3 to 6 carbon atoms. Representative examples of monocyclic cycloalkyls include, but are not limited to, cyclopropyl, cyclobutyl, cyclopentyl, cyclopentenyl, cyclohexyl, cyclohexenyl, cyclohexadienyl, cycloheptyl, cycloheptatrienyl, cyclooctyl, etc. Polycyclic cycloalkyl includes a cycloalkyl having a spiro ring, fused ring or bridged ring.

“Spiro Cycloalkyl” refers to a 5 to 20 membered polycyclic group with rings connected through one common carbon atom (called a spiro atom), wherein one or more rings can contain one or more, preferably one to three, double bonds, it can be aryl and heteroaryl. Preferably a spiro cycloalkyl is 6 to 14 membered, and more preferably 7 to 10 membered (such as 7, 8, 9 and 10 membered). According to the number of common spiro atoms, a spiro cycloalkyl is divided into mono-spiro cycloalkyl, di-spiro cycloalkyl, or poly-spiro cycloalkyl, and preferably refers to a mono-spiro cycloalkyl or di-spiro cycloalkyl, more preferably 4-membered/4-membered, 4-membered/5-membered, 4-membered/6-membered, 5-membered/5-membered, 5-membered/6-membered, 6-membered/6-membered mono-spiro cycloalkyl. Representative examples of spiro cycloalkyl include, but are not limited to the following groups:

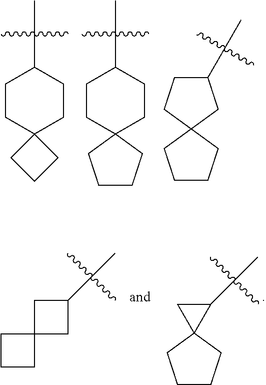

“Fused Cycloalkyl” refers to a polycyclic group, which is a cycloalkyl attached together with one or more, preferably one to five, and sometimes more preferably one to three, group(s) independently selected from cycloalkyl, heterocyclyl, aryl and heteroaryl in a fused manner, wherein cycloalkyl, heterocyclyl, aryl and heteroaryl are as defined in the present disclosure. According to the number of membered rings, fused cycloalkyl is divided into bicyclic, tricyclic, tetracyclic or polycyclic fused cycloalkyl, and preferably refers to a bicyclic or tricyclic fused cycloalkyl, more preferably refers to aryl fused C₅₋₈cycloalkyl, heteroaryl fused C₅₋₈cycloalkyl. 4-membered heterocyclyl fused C₅₋₈ cycloalkyl, 5-membered heterocyclyl fused C₅₋₈ cycloalkyl, C₆ cycloalkyl fused C₅₋₈ cycloalkyl or C₅ cycloalkyl fused C₅₋₈ cycloalkyl, Representative examples of fused cycloalkyls include, but are not limited to, the following groups:

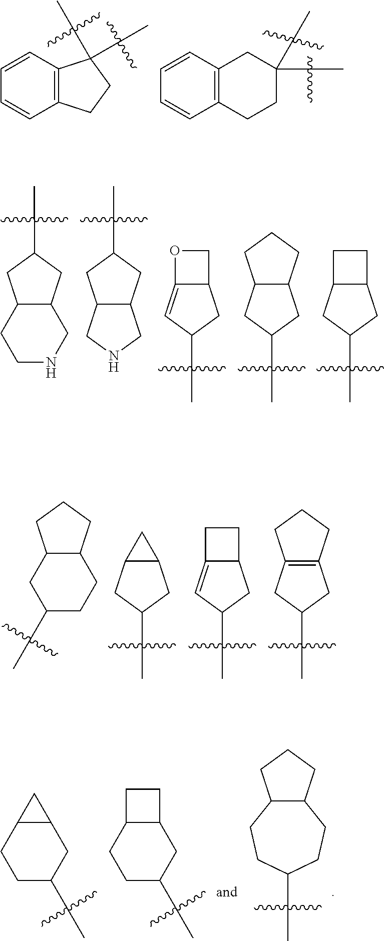

“Bridged Cycloalkyl” refers to a 5 to 20 membered polycyclic hydrocarbon group, wherein every two rings in the system share two disconnected carbon atoms. The rings can have one or more, preferably one to three, double bonds. Preferably, a bridged cycloalkyl is 6 to 14 membered, and more preferably 7 to 10 membered (such as 7, 8, 9 and 10 membered). According to the number of membered rings, bridged cycloalkyl is divided into bicyclic, tricyclic, tetracyclic or polycyclic bridged cycloalkyl, and preferably refers to a bicyclic, tricyclic or tetracyclic bridged cycloalkyl, more preferably a bicyclic or tricyclic bridged cycloalkyl. Representative examples of bridged cycloalkyls include, but are not limited to, the following groups:

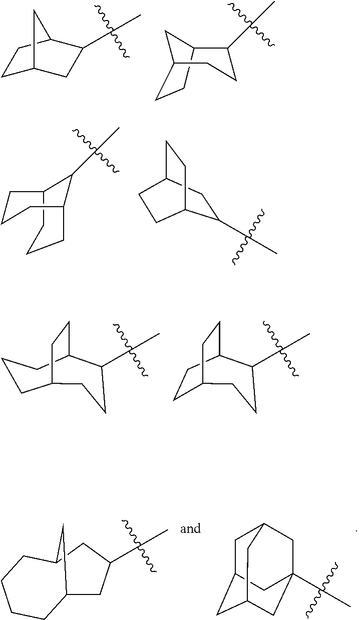

The cycloalkyl can be fused to the ring of an aryl, heteroaryl or heterocyclyl, wherein the ring bound to the parent structure is cycloalkyl. Representative examples include, but are not limited to indanylacetic, tetrahydronaphthalene, benzocycloheptyl and so on. The cycloalkyl is optionally substituted or unsubstituted. When substituted, the substituent group(s) is preferably one or more, sometimes preferably 1 to 5, and sometimes more preferably 1 to 3, groups independently selected from alkyl, halogen, alkoxy, alkenyl, alkynyl, alkylthio, alkylamino, thiol, hydroxy, nitro, cyano, amino, cycloalkyl, heterocyclyl, aryl, heteroaryl, cycloalkoxy, cycloalkylthio, heterocyclylthio and oxo group.

“Heterocyclyl” refers to a 3 to 20 membered saturated and/or partially unsaturated monocyclic or polycyclic hydrocarbon group having one or more, preferably one to five, and sometimes more preferably one to three, heteroatoms selected from N, O, S, S(O) and S(O)₂ as ring atoms, but excluding —O—O—, —O—S— or —S—S— in the ring, the remaining ring atoms being C. Preferably, heterocyclyl is a 3 to 12 membered (such as 3, 4, 5, 6, 7, 8, 9, 10, 11 and 12 membered) having 1 to 4 heteroatoms (such as 1, 2, 3 and 4 heteroatom(s)); more preferably a 3 to 8 membered (such as 3, 4, 5, 6, 7 and 8 membered) having 1 to 3 heteroatoms (such as 1, 2 and 3 heteroatom(s)); even more preferably a 3 to 6 membered (such as 3, 4, 5 and 6 membered) having 1 to 3 heteroatom(s) (such as 1, 2 and 3 heteroatom(s)); most preferably a 5 to 6 membered having 1 to 3 heteroatom(s) (such as 1, 2 and 3 heteroatom(s)). Representative examples of monocyclic heterocyclyls include, but are not limited to, pyrrolidyl, piperidyl, piperazinyl, morpholinyl, sulfo-morpholinyl, homopiperazinyl, and so on. Polycyclic heterocyclyl includes the heterocyclyl having a spiro ring, fused ring or bridged ring.

“Spiro heterocyclyl” refers to a 5 to 20 membered polycyclic heterocyclyl with rings connected through one common carbon atom (called a spiro atom), wherein said rings have one or more heteroatoms selected from N, O, S, S(O) and S(O)₂ as ring atoms, the remaining ring atoms being C, wherein one or more rings can contain one or more double bonds. Preferably a spiro heterocyclyl is 6 to 14 membered (such as 6, 7, 8, 9, 10, 11, 12, 13 and 14 membered), and more preferably 7 to 10 membered. According to the number of common spiro atoms, spiro heterocyclyl is divided into mono-spiro heterocyclyl, di-spiro heterocyclyl, or poly-spiro heterocyclyl, and preferably refers to monospiro heterocyclyl or di-spiro heterocyclyl, more preferably 4-membered/4-membered, 4-membered/5-membered, 4-membered/6-membered, 5-membered/5-membered, 5-membered/6-membered, 6-membered/6-membered mono-spiro heterocyclyl. Representative examples of spiro heterocyclyl include, but are not limited to the following groups:

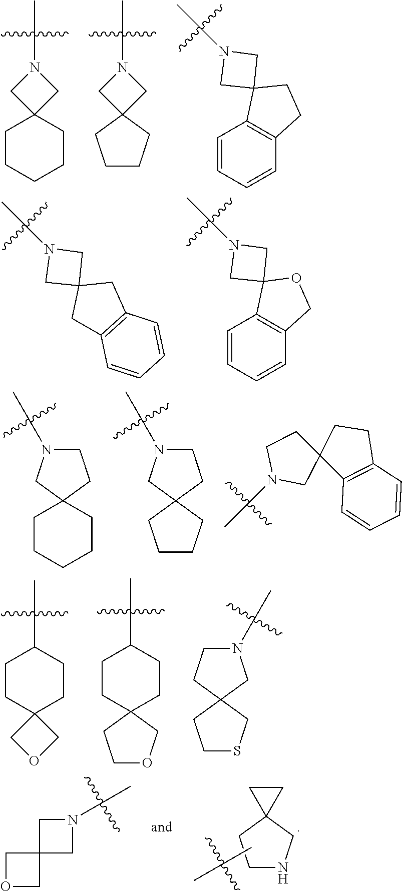

“Fused Heterocyclyl” refers to a polycyclic group, which is a heterocyclyl attached together with one or more, preferably one to three, group(s) selected from cycloalkyl, heterocyclyl, aryl and heteroaryl in a fused manner. Wherein cycloalkyl, heterocyclyl, aryl and heteroaryl are as defined in the present disclosure. According to the number of membered rings, fused heterocyclyl is divided into bicyclic, tricyclic, tetracyclic or polycyclic fused heterocyclyl, and preferably refers to a bicyclic or tricyclic fused cycloalkyl, more preferably refers to aryl fused 5 to 8-member heterocyclyl, heteroaryl fused 5 to 8-member heterocyclyl. C₅₋₈ cycloalkyl fused 4-membered heterocyclyl, C₅₋₈ cycloalkyl fused 5-membered heterocyclyl, C₅₋₈ cycloalkyl fused 6-member heterocyclyl. Representative examples of fused heterocyclyl include, but are not limited to, the following groups:

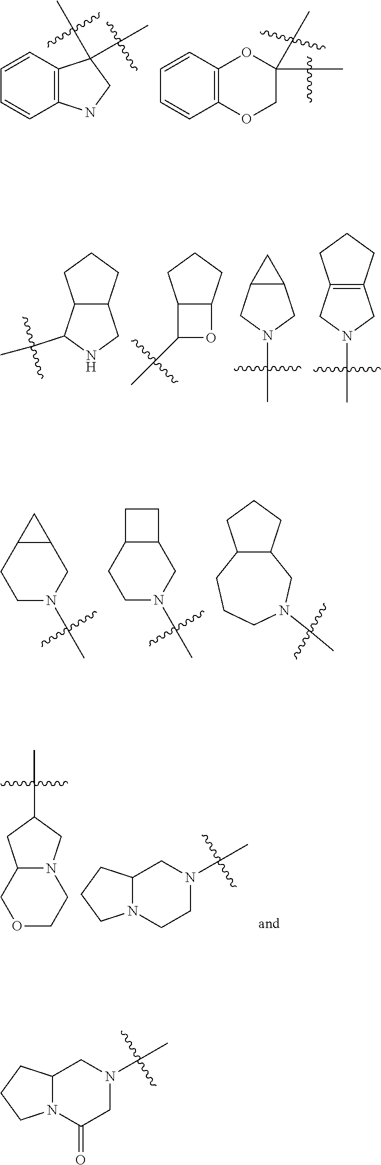

“Bridged Heterocyclyl” refers to a 5 to 14 membered (such as 5, 6, 7, 8, 9, 10, 11, 12, 13 and 14 membered) polycyclic heterocyclyl group, wherein every two rings in the system share two disconnected atoms, the rings can have one or more, preferably one to three, double bonds, and the rings have one or more, preferably one to five, and sometimes more preferably one to three, heteroatoms independently selected from N, O, S, S(O) and S(O)₂ as ring atoms, the remaining ring atoms being C. Preferably a bridged heterocyclyl is 6 to 14 membered (such as 6, 7, 8, 9, 10, 11, 12, 13 and 14 membered), and more preferably 7 to 10 membered. According to the number of membered rings, bridged heterocyclyl is divided into bicyclic, tricyclic, tetracyclic or polycyclic bridged heterocyclyl, and preferably refers to bicyclic, tricyclic or tetracyclic bridged heterocyclyl, more preferably bicyclic or tricyclic bridged heterocyclyl. Representative examples of bridged heterocyclyl include, but are not limited to, the following groups:

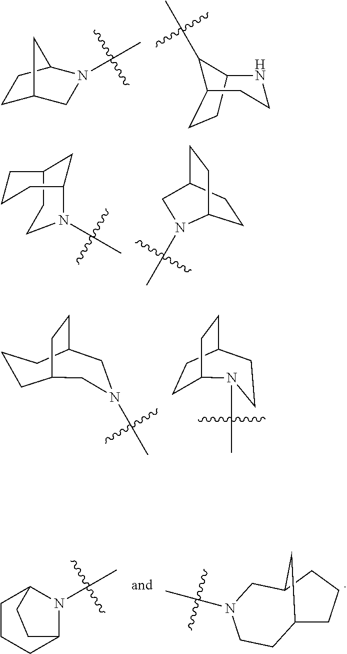

The ring of said heterocyclyl can be fused to the ring of an aryl, heteroaryl or cycloalkyl, wherein the ring bound to the parent structure is heterocyclyl. Representative examples include, but are not limited to the following groups:

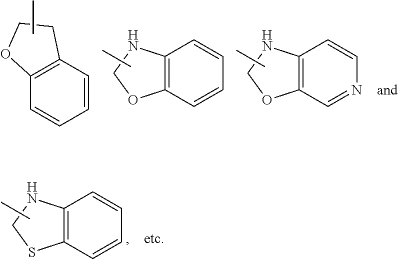

The heterocyclyl is optionally substituted or unsubstituted. When substituted, the substituent group(s) is preferably one or more, sometimes preferably 1 to 5 (such as 1, 2, 3, 4 and 5), and sometimes more preferably 1 to 3, group(s) independently selected from alkyl, alkenyl, alkynyl, alkoxy, alkylthio, alkylamino, halogen, thiol, hydroxy, nitro, cyano, cycloalkyl, heterocyclyl, aryl, heteroaryl, cycloalkoxy, heterocyclyloxy, cycloalkylthio, heterocyclylthio and oxo group.

“Aryl” refers to a 6 to 14 membered (such as 6, 7, 8, 9, 10, 11, 12, 13 and 14 membered) all-carbon monocyclic ring or a polycyclic fused ring (a “fused” ring system means that each ring in the system shares an adjacent pair of carbon atoms with another ring in the system) group, and has a completely conjugated pi-electron system. Preferably aryl is 6 to 10 membered (such as 6, 7, 8, 9 and 10 membered), such as phenyl and naphthyl, most preferably phenyl. The aryl can be fused to the ring of heteroaryl, heterocyclyl or cycloalkyl, wherein the ring bound to parent structure is aryl. Representative examples include, but are not limited to, the following groups:

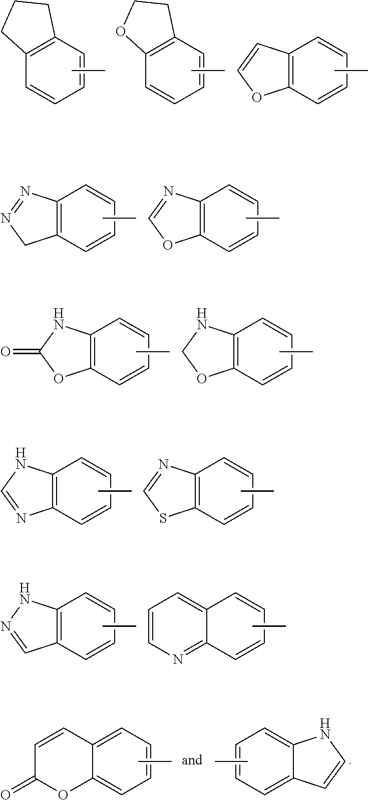

The aryl group can be substituted or unsubstituted. When substituted, the substituent group(s) is preferably one or more, sometimes preferably 1 to 5, and sometimes more preferably 1 to 3, group(s) independently selected from alkyl, alkenyl, alkynyl, alkoxy, alkylthio, alkylamino, halogen, thiol, hydroxy, nitro, cyano, cycloalkyl, heterocyclyl, aryl, heteroaryl, cycloalkoxy, heterocyclyloxy, cycloalkylthio, heterocyclylthio and oxo group.

“Heteroaryl” refers to an aryl system having 1 to 4 heteroatom(s) (such as 1, 2, 3 and 4 heteroatom(s)) selected from O, S and N as ring atoms and having 5 to 14 annular atoms (such as 5, 6, 7, 8, 9, 10, 11, 12, 13 and 14). Preferably a heteroaryl is 5- to 10-membered (such as 5, 6, 7, 8, 9 and 10 membered), more preferably 5- or 6-membered, for example, thiadiazolyl, pyrazolyl, oxazolyl, oxadiazolyl, imidazolyl, triazolyl, thiazolyl, furyl, thienyl, pyridyl, pyrrolyl, N-alkyl pyrrolyl, pyrimidinyl, pyrazinyl, imidazolyl, tetrazolyl, and the like. The heteroaryl can be fused with the ring of an aryl, heterocyclyl or cycloalkyl, wherein the ring bound to parent structure is heteroaryl. Representative examples include, but are not limited to, the following groups:

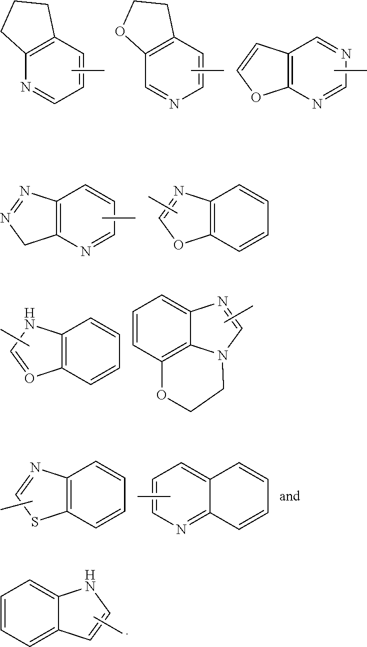

The heteroaryl group can be substituted or unsubstituted. When substituted, the substituent group(s) is preferably one or more, sometimes preferably 1 to 5 (such as 1, 2, 3, 4 and 5), and sometimes more preferably 1 to 3, group(s) independently selected from alkyl, alkenyl, alkynyl, alkoxy, alkylthio, alkylamino, halogen, thiol, hydroxy, nitro, cyano, cycloalkyl, heterocyclyl, aryl, heteroaryl, cycloalkoxy, heterocyclyloxy, cycloalkylthio, heterocyclylthio and oxo group.

“Alkoxy” refers to both an —O-(alkyl) and an —O-(unsubstituted cycloalkyl) group, wherein the alkyl is defined as above. Representative examples include, but are not limited to, methoxy, ethoxy, propoxy, butoxy, cyclopropyloxy, cyclobutyloxy, cyclopentyloxy, cyclohexyloxy, and the like. The alkoxyl can be substituted or unsubstituted. When substituted, the substituent is preferably one or more, sometimes preferably 1 to 5 (such as 1, 2, 3, 4 and 5), and sometimes more preferably 1 to 3, group(s) independently selected from alkyl, alkenyl, alkynyl, alkoxy, alkylthio, alkylamino, halogen, thiol, hydroxy, nitro, cyano, cycloalkyl, heterocyclyl, aryl, heteroaryl, cycloalkoxy, heterocyclyloxy, cycloalkylthio, heterocyclylthio and oxy group.

“Amino protecting group” refers to a derivative of the groups commonly employed to block or protect an amino group while reactions are carried out on other functional groups on the compound. Examples of such protecting groups include carbamates, amides, alkyl and aryl groups, and imines, as well as many N-heteroatom derivatives which can be removed to regenerate the desired amine group. Non-limiting examples include (trimethylsilyl)ethoxymethyl (SEM), tetrahydropyranyl, tert-butoxycarbonyl (Boc), benzyloxycarbonyl (Cbz), (9-Fluorenylmethyloxycarbonyl) (Fmoc), acetyl, benzyl, allyl Group and p-methoxybenzyl (Pmb), etc.

“Hydroxyl protecting group” refers to a derivative of the hydroxy group commonly employed to block or protect the hydroxy group while reactions are carried out on other functional groups on the compound. Examples of such protecting groups include triethylsilyl, triisopropylsilyl, tert-butyldimethylsilyl Group (TBS), tert-butyldiphenylsilyl, etc.; or C₁₋₁₀ alkyl or substituted alkyl, preferably alkoxy or aryl substituted alkyl, more preferably C₁₋₆ alkoxy substituted C₁₋₆ alkyl or phenyl substituted C₁₋₆ alkyl, most preferably C₁₋₄ alkoxy substituted C₁₋₄ alkyl, for example: methyl, tert-butyl, allyl, benzyl, methoxy Methyl (MOM), ethoxyethyl, 2-tetrahydropyranyl (THP), etc.; or (C₁₋₁₀ alkyl or aryl) acyl, such as formyl, acetyl, benzoyl, P-nitrobenzoyl, etc.; (C₁₋₆ alkyl or C₆₋₁₀ aryl)sulfonyl; or (C₁₋₆ alkoxy or C₆₋₁₀ aryloxy) carbonyl.

“Bond” refers to a covalent bond using a sign of “—”.

“deuterated alkyl” refers to an alkyl group substituted by a or more deuterium atom, wherein alkyl is as defined above.

“Hydroxyalkyl” refers to an alkyl group substituted by a hydroxy group, wherein alkyl is as defined above.

“Hydroxy” refers to an —OH group.

“Halogen” refers to fluoro, chloro, bromo or iodo atoms.

“Amino” refers to a —NH₂ group.

“Cyano” refers to a —CN group.

“Nitro” refers to a —NO₂ group.

“Oxo group” refers to a ═O group.

“Carboxyl” refers to a —C(O)OH group.

“Alkoxycarbonyl” refers to a —C(O)O(alkyl) or (cycloalkyl) group, wherein the alkyl and cycloalkyl are defined as above.

“Optional” or “optionally” means that the event or circumstance described subsequently can, but need not, occur, and the description includes the instances in which the event or circumstance may or may not occur. For example, “the heterocyclyl group optionally substituted by an alkyl” means that an alkyl group can be, but need not be, present, and the description includes the case of the heterocyclyl group being substituted with an alkyl and the heterocyclyl group being not substituted with an alkyl.

“Substituted” refers to one or more hydrogen atoms in the group, preferably up to 5, more preferably 1 to 3 hydrogen atom(s), independently substituted with a corresponding number of substituents. The person skilled in the art is able to determine if the substitution is possible or impossible without paying excessive efforts by experiment or theory. For example, the combination of amino or hydroxyl group having free hydrogen and carbon atoms having unsaturated bonds (such as olefinic) may be unstable.

A “pharmaceutical composition” refers to a mixture of one or more of the compounds described in the present disclosure or physiologically/pharmaceutically acceptable salts or prodrugs thereof and other chemical components such as physiologically/pharmaceutically acceptable carriers and excipients. The purpose of a pharmaceutical composition is to facilitate administration of a compound to an organism, which is conducive to the absorption of the active ingredient and thus displaying biological activity.

“Pharmaceutically acceptable salts” refer to salts of the compounds of the disclosure, such salts being safe and effective when used in a mammal and have corresponding biological activity.

The salts can be prepared during the final isolation and purification of the compounds or separately by reacting a suitable nitrogen atom with a suitable acid. Acids commonly employed to form pharmaceutically acceptable salts include inorganic acids such as hydrochloric acid, hydrobromic acid, hydroiodic acid, sulfuric acid, phosphoric acid, hydrogen bisulfide as well as organic acids, such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid, acetic acid, and related inorganic and organic acids.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of pharmaceutically acceptable salts include, but are not limited to, lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, and N-methylmorpholine.

As a person skilled in the art would understand, the compounds of formula (I) or Pharmaceutically acceptable salts thereof disclosed herein may exist in prodrug or solvate forms, which are all encompassed by the present disclosure.

“Prodrug” refers to compounds that can be transformed in vivo to yield the active parent compound under physiological conditions, such as through hydrolysis in blood. Common examples include, but are not limited to, ester and amide forms of a compound having an active form bearing a carboxylic acid moiety. Amides and esters of the compounds of the present disclosure may be prepared according to conventional methods. In particular, in the present disclosure, a prodrug may also be formed by acylation of an amino group or a nitrogen atom in a heterocyclyl ring structure, which acyl group can be hydrolyzed in vivo. Such acyl group includes, but is not limited to, a C₁-C₆ acyl, preferably C₁-C₄ acyl, and more preferably C₁-C₂ (formyl or acetyl) group, or benzoyl.

The term “solvate,” as used herein, means a physical association of a compound of this disclosure with one or more, preferably one to three, solvent molecules, whether organic or inorganic. This physical association includes hydrogen bonding. In certain instances the solvate will be capable of isolation, for example, when one or more, preferably one to three, solvent molecules are incorporated in the crystal lattice of the crystalline solid. Exemplary solvates include, but are not limited to, hydrates, ethanolates, methanolates, and isopropanolates. Methods of solvation are generally known in the art.

The compounds of the present disclosure may also contain unnatural proportions of atomic isotopes at one or more of the atoms that constitute such compounds. Unnatural proportions of an isotope may be defined as ranging from the amount found in nature to an amount consisting of 100% of the atom in question. For example, the compounds may incorporate radioactive isotopes, such as for example tritium (³H), iodine-125 (¹²⁵I) or carbon-14 (¹⁴C), or non-radioactive isotopes, such as deuterium (D) or carbon-13 (¹³C). Such isotopic variations can provide additional utilities to those described elsewhere within this application. For instance, isotopic variants of the compounds of the disclosure may find additional utility, including but not limited to, as diagnostic and/or imaging reagents, or as cytotoxic/radiotoxic therapeutic agents.

The phrase “therapeutically effective amount” refers to the administration of an agent to a subject, either alone or as part of a pharmaceutical composition and either in a single dose or as part of a series of doses, in an amount capable of having any detectable, positive effect on any symptom, aspect, or characteristic of a disease, disorder or condition when administered to the subject. The therapeutically effective amount can be ascertained by measuring relevant physiological effects, and it can be adjusted in connection with the dosing regimen and diagnostic analysis of the subject's condition, and the like. By way of example, measurement of the serum level of a RAF inhibitor (or, e.g., a metabolite thereof) at a particular time post-administration may be indicative of whether a therapeutically effective amount has been used.

The term “pharmaceutically acceptable,” as used herein, refers to those compounds, materials, compositions, and/or dosage forms which are, within the scope of sound medical judgment, suitable for use in contact with the tissues of patients without excessive toxicity, irritation, allergic response, or other problem or complication commensurate with a reasonable benefit/risk ratio, and are effective for their intended use.

The term “treat”, “treating”, “treatment”, or the like, refers to: (i) inhibiting the disease, disorder, or condition, i.e., arresting its development; and (ii) relieving the disease, disorder, or condition, i.e., causing regression of the disease, disorder, and/or condition. In addition, the compounds of present disclosure may be used for their prophylactic effects in preventing a disease, disorder or condition from occurring in a subject that may be predisposed to the disease, disorder, and/or condition but has not yet been diagnosed as having it.

The term “subject” or “patient” refers to a mammalian animal.

The term “mammal” or “mammalian animal” includes, but is not limited to, humans, dogs, cats, horses, pigs, cows, monkeys, rabbits and mice. The preferred mammals are humans.

As used herein, the singular forms “a”, “an”, and “the” include plural reference, and vice versa, unless the context clearly dictates otherwise.

When the term “about” is applied to a parameter, such as pH, concentration, temperature, or the like, it indicates that the parameter can vary by ±10%, and sometimes more preferably within ±5%. As would be understood by a person skilled in the art, when a parameter is not critical, a number is often given only for illustration purpose, instead of being limiting.

### Synthesis Method of the Compound of the Present Disclosure

In order to complete the purpose of the disclosure, the present disclosure applies, but is not limited to, the following technical solution:

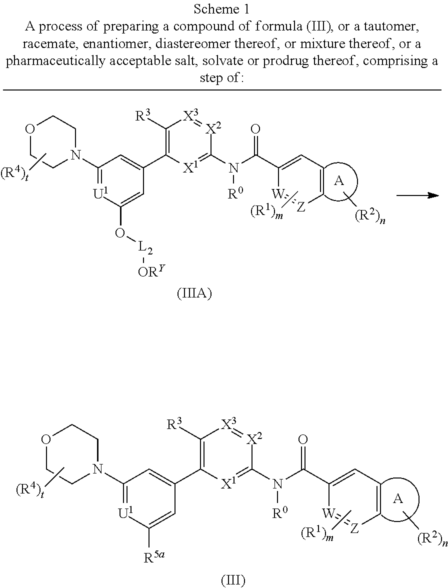

removing the hydroxyl protecting group of Formula (IIIA) or a tautomer, racemate, enantiomer, diastereomer thereof, or mixture thereof, or a pharmaceutically acceptable salt, solvate or prodrug thereof, to obtain the compound of formula (III) or a tautomer, racemate, enantiomer, diastereomer thereof, or mixture thereof, or a pharmaceutically acceptable salt, solvate or prodrug thereof, under acidic or alkaline conditions, wherein:

L₂ is alkylene; preferably L₂ is C₁₋₆ alkylene; more preferably L₂ is CH₂;

R^{Y} is a hydroxyl protecting group; preferably R^{Y} is selected from TBS and THP;

R^{5a} is —O-L₂-OH; preferably R^{5a} is —O—(CH₂)₁₋₆—OH; more preferably R^{5a} is —O—(CH₂)₂—OH; and

W, Z, R⁰, X¹, X², X³, U¹, ring A, R¹ to R⁴, m, n and t are as defined in formula (III).

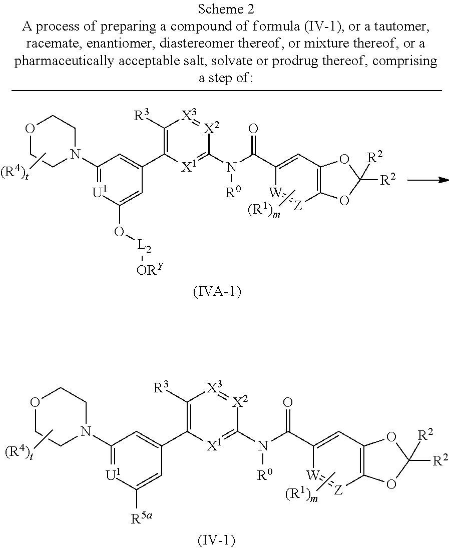

removing the hydroxyl protecting group of Formula (IVA-1) or a tautomer, racemate, enantiomer, diastereomer thereof, or mixture thereof, or a pharmaceutically acceptable salt, solvate or prodrug thereof, to obtain the compound of formula (IV-1) or a tautomer, racemate, enantiomer, diastereomer thereof, or mixture thereof, or a pharmaceutically acceptable salt, solvate or prodrug thereof, under acidic or alkaline conditions;
wherein:

L₂ is alkylene; preferably L₂ is C₁₋₆ alkylene; more preferably L₂ is CH₂;

R^{Y} is a hydroxyl protecting group; preferably R^{Y} is selected from TBS and THP;

R^{5a} is —O-L₂-OH; preferably R^{5a} is —O—(CH₂)₁₋₆—OH; more preferably R^{5a} is —O—(CH₂)₂—OH; and

W, Z, R⁰, X¹, X², X³, U¹, R¹ to R⁴, m and t are as defined in formula (IV-1).

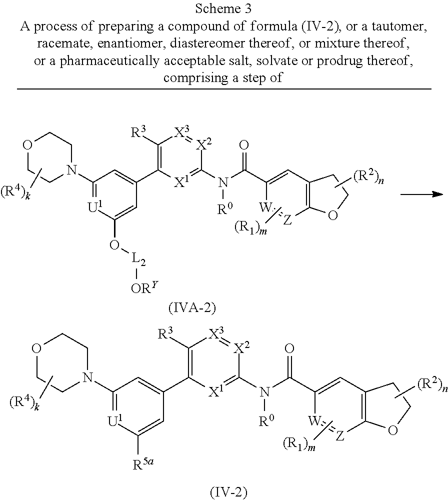

removing the hydroxyl protecting group of Formula (IVA-2) or a tautomer, racemate, enantiomer, diastereomer thereof, or mixture thereof, or a pharmaceutically acceptable salt, solvate or prodrug thereof, to obtain the compound of formula (IV-2) or a tautomer, racemate, enantiomer, diastereomer thereof, or mixture thereof, or a pharmaceutically acceptable salt, solvate or prodrug thereof, under acidic or alkaline conditions, wherein:

L₂ is alkylene; preferably L₂ is C₁₋₆ alkylene; more preferably L₂ is CH₂;

R^{Y} is a hydroxyl protecting group; preferably R^{Y} is selected from TBS and THP;

R^{5a} is —O-L₂-OH; preferably R^{5a} is —O—(CH₂)₁₋₆—OH; more preferably R^{5a} is —O—(CH₂)₂—OH; and

W, Z, R⁰, X¹, X², X³, R¹ to R⁴, m, n and t are as defined in formula (IV-2).

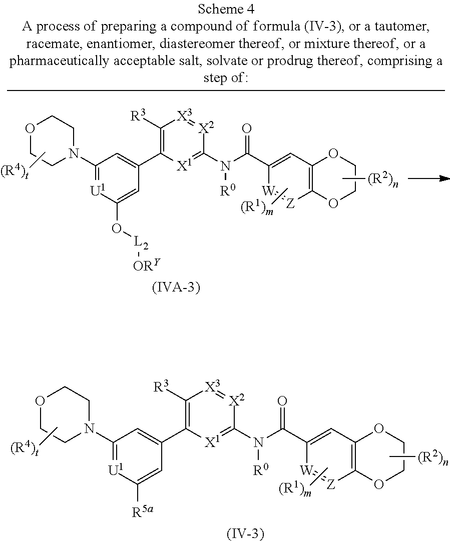

removing the hydroxyl protecting group of Formula (IVA-3) or a tautomer, racemate, enantiomer, diastereomer thereof, or mixture thereof, or a pharmaceutically acceptable salt, solvate or prodrug thereof, to obtain the compound of formula (IV-3) or a tautomer, racemate, enantiomer, diastereomer thereof, or mixture thereof, or a pharmaceutically acceptable salt, solvate or prodrug thereof, under acidic or alkaline conditions, wherein:

L₂ is alkylene; preferably L₂ is C₁₋₆ alkylene; more preferably L₂ is CH₂;

R^{Y} is a hydroxyl protecting group; preferably R^{Y} is selected from TBS and THP;

R^{5a} is —O-L₂-OH; preferably R^{5a} is —O—(CH₂)₁₋₆—OH; more preferably R^{5a} is —O—(CH₂)₂—OH; and

W, Z, R⁰, X¹, X², X³, R¹ to R⁴, m, n and t are as defined in formula (IV-3).

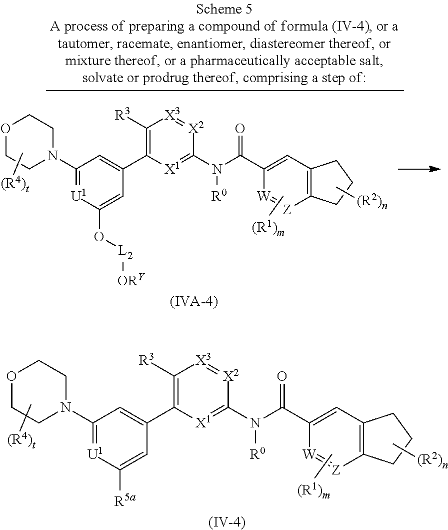

removing the hydroxyl protecting group of Formula (IVA-4) or a tautomer, racemate, enantiomer, diastereomer thereof, or mixture thereof, or a pharmaceutically acceptable salt, solvate or prodrug thereof, to obtain the compound of formula (IV-4) or a tautomer, racemate, enantiomer, diastereomer thereof, or mixture thereof, or a pharmaceutically acceptable salt, solvate or prodrug thereof, under acidic or alkaline conditions, wherein:

L₂ is alkylene; preferably L₂ is C₁₋₆ alkylene; more preferably L₂ is CH₂;

R^{Y} is a hydroxyl protecting group; preferably R^{Y} is selected from TBS and THP;

R^{5a} is —O-L₂-OH; preferably R^{5a} is —O—(CH₂)₁₋₆—OH; more preferably R^{5a} is —O—(CH₂)₂—OH; and

W, Z, R⁰, X¹, X², X³, U¹, R¹ to R⁴, m, n and t are as defined in formula (IV-4).

The agent which provides the acidic condition includes organic acids and inorganic acids, wherein the organic acid includes, but is not limited to, trifluoroacetic acid, formic acid, acetic acid, methanesulfonic acid, p-toluenesulfonic acid, Me₃SiCl and TMSOTf, preferably trifluoroacetic acid; the inorganic acid includes, but not limited to hydrogen chloride, 1,4-dioxane solution of hydrogen chloride, hydrochloric acid, sulfuric acid, nitric acid and phosphoric acid, preferably hydrochloric acid.

The agent which provides the alkaline condition includes organic bases and inorganic bases, wherein the organic base includes, but is not limited to, triethylamine, N,N-diisopropylethylamine (DIEA), n-butyllithium, lithium diisopropylamide, potassium acetate, sodium tert-butoxide and potassium tert-butoxide, and wherein the inorganic base includes, but is not limited to, sodium hydride, potassium phosphate, sodium carbonate, potassium carbonate and cesium carbonate, preferably sodium hydride.

The reaction is preferably in solvent, wherein solvent used herein includes, but is not limited to, acetic acid, methanol, ethanol, toluene, acetone, tetrahydrofuran, dichloromethane, dichloroethane, dim ethyl sulfoxide, 1,4-dioxane, water, N,N-dimethylformamide, trimethylphosphate, methyl tert-butyl ether, pyridine and the mixture thereof.

### EXAMPLES

The following examples serve to illustrate the disclosure, but the examples should not be considered as limiting the scope of the disclosure. If specific conditions for the experimental method are not specified in the examples of the present disclosure, they are generally in accordance with conventional conditions or recommended conditions of the raw materials and the product manufacturer. The reagents without a specific source indicated are commercially available, conventional reagents.

The structure of each compound was identified by nuclear magnetic resonance (NMR) and/or mass spectrometry (MS). NMR chemical shifts (δ) were given in 10-6 (ppm). NMR was determined by Bruker AVANCE-300, AVANCE-400 or AVANCE-500 machine. The solvents were deuterated-dimethyl sulfoxide (DMSO-d6), deuterated-chloroform (CDCl₃) and deuterated methanol (CD₃OD).

High performance liquid chromatography (HPLC) was determined on an Agilent 1200DAD high pressure liquid chromatography spectrometer (Sunfire C18 150×4.6 mm chromatographic column), a Waters 2695-2996 high pressure liquid chromatography spectrometer (Gimini C18 150×4.6 mm chromatographic column), or Shimadzu UFLC equipped with an Xbridge C18 (5 um 150×4.6 mm) column.

Chiral high performance liquid chromatography (HPLC) is determined on LC-10A vp (Shimadzu) or SFC-analytical (Berger Instruments Inc.) or a Waters-UPC² instrument.

MS is determined by a SHIMADZU (ESI) liquid chromatography-mass spectrometer (manufacturer: Shimadzu, type: LC-20AD, LCMS-2020), Waters UPLC-QDa equipped with an ACQUITY UPLC® BEH (2.1*50 mm 1.7 um) column, or Agilent Agilent6120 equipped with a Xbridge C18 (5 um 50×4.6 mm) column.

The average rates of kinase inhibition, and the IC50 values were determined by Microplate reader (BMG company, Germany).

The thin-layer silica gel plates used in thin-layer chromatography were Yantai Huanghai HSGF254 or Qingdao GF254 silica gel plate. The dimension of the plates used in TLC was 0.15 mm to 0.2 mm, and the dimension of the plates used in thin-layer chromatography for product purification was 0.4 mm to 0.5 mm.

Column chromatography generally used Yantai Huanghai 200 to 300 mesh silica gel as carrier.

The known starting material of the disclosure can be prepared by the conventional synthesis method in the prior art, or can be purchased from ABCR GmbH & Co. KG, Acros Organics, Aldrich Chemical Company, Accela ChemBio Inc., Dari chemical Company, Fisher Scientific or Combi-Blocks, etc.

Unless otherwise stated in the examples, the following reactions were placed under argon atmosphere or nitrogen atmosphere.

The term “argon atmosphere” or “nitrogen atmosphere” means that a reaction flask was equipped with a balloon having 1 L of argon or nitrogen.

The term “hydrogen atmosphere” means that a reaction flask was equipped with a balloon having 1 L of hydrogen.

High pressure hydrogenation reactions were performed with a Parr 3916EKX hydrogenation apparatus and clear blue QL-500 hydrogen generator or HC2-SS hydrogenation apparatus. In hydrogenation reactions, the reaction system was generally vacuumed and filled with hydrogen, and the above operation was repeated three times.

Microwave reactions were performed with a CEM Discover-S 908860 microwave reactor. Unless otherwise stated in the examples, the solution used in following reactions refers to an aqueous solution.

Unless otherwise stated in the examples, the reaction temperature in the following reactions was room temperature.

Unless otherwise stated, the reaction temperature in the reactions refers to room temperature, and the range of the temperature was 20° C. to 30° C.

The reaction process is monitored by LC-MS or thin layer chromatography (TLC), and the developing solvent system includes: A: dichloromethane and methanol, B: hexane and ethyl acetate. The ratio of the volume of the solvent was adjusted according to the polarity of the compounds. The elution system for purification of the compounds by column chromatography, thin layer chromatography and CombiFlash flash rapid preparation instrument includes: A: dichloromethane and methanol, B: hexane and ethyl acetate. The ratio of the volume of the solvent can be adjusted according to the polarity of the compounds, and sometimes a small amount of basic reagent such as ammonia or acidic reagent such as acetic acid can be added.

Final compounds are purified by Shimadzu (LC-20AD, SPD20A) Preparative HPLC (Phenomenex Gemini-NX 5 uM C18 21.2×100 mm column), Waters 2767 equipped with a Sunfire Pre C18 (10 um 19×250 mm) column, or Waters 2767-QDa equipped with an Xbridge Pre C18 (10 um 19×250 mm) column instrument, with water/MeOH or water/CH₃CN elution systems with optional additives, such as HCOOH, TFA.

Pre-SFC was performed on a Waters-SFC80 equipped with Daciel AD/OD/OJ/IC/IA/ID (10 um 20×250 mm) column instrument.

CombiFlash was performed on systems from Teledyne ISCO or Agela Technologies. The following abbreviations are used:

TEA is triethylamine;

HATU is 1-[Bis(dimethylamino)methylene]-1H-1,2,3-triazolo[4,5-b]pyridinium 3-oxide hexafluorophosphate;

HBTU is O-(Benzotriazol-1-yl)-N,N,N′,N′-tetramethyluronium hexafluorophosphate;

DCM is dichloromathene;

DMF is N,N-dimethylformamide;

DMSO is dimethyl sulfoxide;

DEAD is diethyl azodiformate;

EtOAc is ethyl acetate;

Prep HPLC is Preparative high performance liquid chromatography;

NMR is proton nuclear magnetic resonance; and

MS is mass spectroscopy with (+) referring to the positive mode which generally gives a M+H (or M+H) absorption where M=the molecular mass.

### Synthesis of Intermediate

### 3-(2-fluoro-6-morpholinopyridin-4-yl)-4-methylaniline Int-1

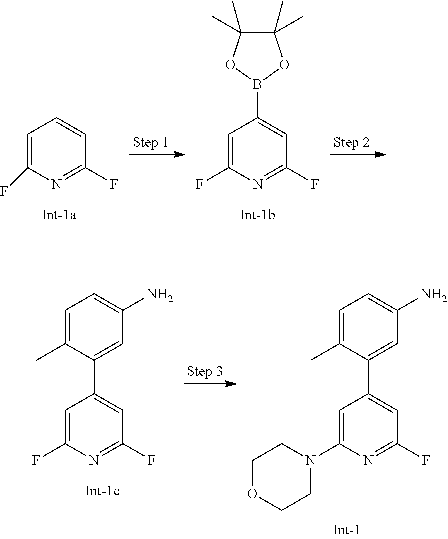 [SMILES: Fc1cccc(F)n1 ; CC1(C)OB(c2cc(F)nc(F)c2)OC1(C)C ; Cc1ccc(N)cc1-c1cc(F)nc(F)c1 ; Cc1ccc(N)cc1-c1cc(F)nc(N2CCOCC2)c1]

### Step 1

### 2,6-difluoro-4-(4,4,5,5-tetramethyl-1,3,2-dioxaborolan-2-yl)pyridine Int-1b

Under an atmosphere of nitrogen, a mixture of 2,6-difluoropyridine Int-1a (10.00 g, 86.90 mmol), bis(pinacolato)diboron (26.48 g, 104.27 mmol), chlorobis(cyclooctene)diiridium(I) dimer (466 mg, 695 μmol) and 1,10-phenanthroline (625 mg, 3.48 mmol) was treated with dichloroethane (50 mL). The mixture was heated to 100° C. for 16 h. The mixture was concentrated and the residue was purified by silica gel chromatagraphy (CH₂Cl₂) to give target Int-1b (13.00 g, 62.07% yield). MS m/z (ESI): 242.2 [M+H]⁺

### Step 2

### 3-(2,6-difluoropyridin-4-yl)-4-methylaniline Int-1c

The mixture of Int-1b (500 mg, 2.07 mmol), 3-iodo-4-methylaniline (531 mg, 2.28 mmol), Pd(dppf)Cl₂ (114 mg, 207.43 μmol) and K₂CO₃ (858 mg, 6.22 mmol) in water (2 mL) and dioxane (10 mL) was stirred under Ar at 100° C. for 1 h. The mixture was concentrated, and the residue was purified by silica gel chromatagraphy (CH₂Cl₂) to give target Int-1c (400 mg, 87.56% yield). MS m/z (ESI): 221.4 [M+H]⁺

### Step 3

### 3-(2-fluoro-6-morpholinopyridin-4-yl)-4-methylaniline Int-1

To a solution of Int-1c (4.30 g, 19.53 mmol) in DMSO (30 mL) was added morpholine (5.10 g, 58.58 mmol) and K₂CO₃ (11.23 g, 48.82 mmol). The mixture was heated to 45° C. for 6 h. The mixture was diluted with water and extracted with EtOAc, the organic solution was dried and concentrated to give crude Int-1 (4.5 g, 80.21% yield). MS m/z (ESI): 288.4 [M+H]⁺

### 4-methyl-3-(2-morpholino-6-(2-((tetrahydro-2H-pyran-2-yl)oxy)ethoxy)pyridin-4-yl)aniline

### Int-2

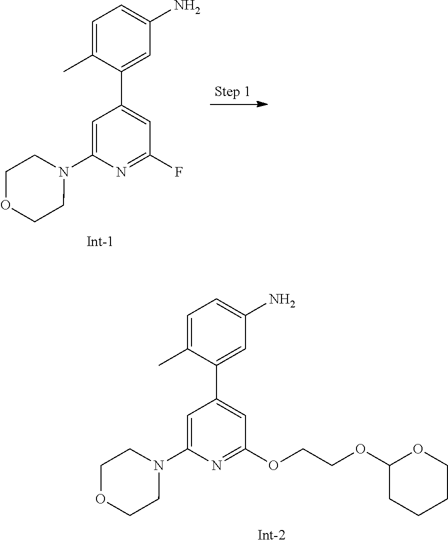 [SMILES: Cc1ccc(N)cc1-c1cc(F)nc(N2CCOCC2)c1 ; Cc1ccc(N)cc1-c1cc(OCCOC2CCCCO2)nc(N2CCOCC2)c1]

### Step 1

To a solution of Int-1 (478 mg, 1.67 mmol) and 2-(tetrahydro-2H-pyran-2-yloxy)ethanol (0.49 mL, 3.34 mmol) in 1,4-dioxane (5 mL), NaH (60% wt, 268 mg, 6.68 mmol) was slowly added at RT. After 30 min, the mixture was heated to 70° C. overnight. The mixture was diluted with water and extracted with EtOAc, the organic solution was dried and purified by silica gel chromatagraphy (EtOAc/hexane) to give target compound Int-2 (496 mg, 72% yield). MS m/z (ESI): 414.2 [M+H]⁺

### Example 1

### 2,2-difluoro-N-(3-(2-(2-hydroxyethoxy)-6-morpholinopyridin-4-yl)-4-methylphenyl)benzo[d][1,3]dioxole-5-carboxamide 1

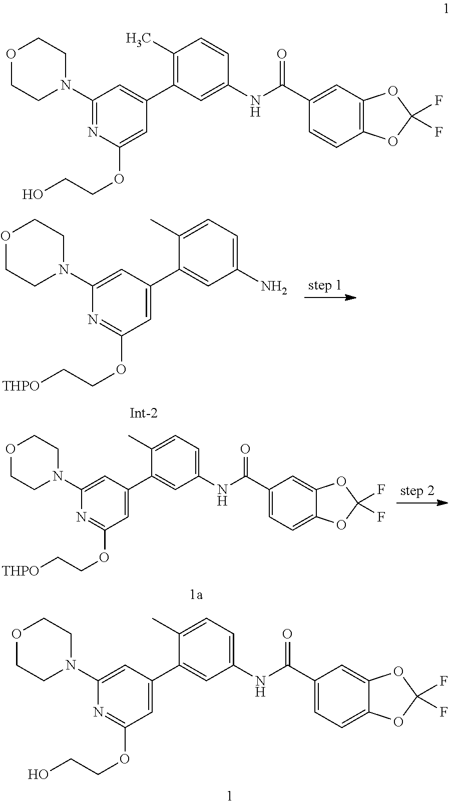

### Step 1

### 2,2-difluoro-N-(4-methyl-3-(2-morpholino-6-(2-((tetrahydro-2H-pyran-2-yl)oxy)ethoxy)pyridin-4-yl)phenyl)benzo[d][1,3]dioxole-5-carboxamide 1a

To a solution of Int-2 (827 mg, 2.00 mmol), 2,2-difluoro-1,3-benzodioxole-5-carboxylic acid (405 mg, 2.00 mmol, Combi-Blocks) and DIPEA (0.70 mL, 4 mmol) in DMF (5 mL) was added HATU (760 mg, 2.00 mmol), then the mixture was stirred at RT for 2 hr. The mixture was diluted with water and extracted with EtOAc. The organic solution was concentrated, and the residue was purified by silica gel chromatagraphy (EtOAc/hexane) to give target 6 (1010 mg, 84.6% yield). MS m/z (ESI): 598.2 [M+H]⁺

### Step 2

### 2,2-difluoro-N-(3-(2-(2-hydroxyethoxy)-6-morpholinopyridin-4-yl)-4-methylphenyl)benzo[d][1,3]dioxole-5-carboxamide 1

To a solution of 1a (825 mg, 1.38 mmol) in THF (10 mL) was added 1.2N aq. HCl (5.5 mL, 6.6 mmol) slowly at 0° C. Then the mixture was stirred at RT for 2 hr. The mixture was cooled at 0° C. again and carefully neutralized by saturated aq. NaHCO₃, then extracted with EtOAc. The organic solution was dried and concentrated. The resulted residue was purified by silica gel chromatagraphy (0-60% EtOAc/hexane) to give target 1 (350 mg, 84.6% yield). ¹H NMR (400 MHz, DMSO-d₆): δ 10.28 (s, 1H), 7.97 (d, 1H), 7.87 (dd, 1H), 7.69 (dd, 1H), 7.63 (d, 1H), 7.58 (d, 1H), 7.27 (d, 1H), 6.25 (s, 1H), 6.03 (s, 1H), 4.80 (t, 1H), 4.26 (t, 2H), 3.73-3.68 (m, 6H), 3.47-3.44 (m, 4H), 2.22 (s, 3H). ¹⁹F NMR (376.5 MHz, DMSO-d₆): δ −48.81. MS m/z (ESI): 514.1 [M+H]⁺.

### Example 2

### N-(3-(2-(2-hydroxyethoxy)-6-morpholinopyridin-4-yl)-4-methylphenyl)bicyclo[4.2.0]octa-1(6),2,4-triene-3-carboxamide 2

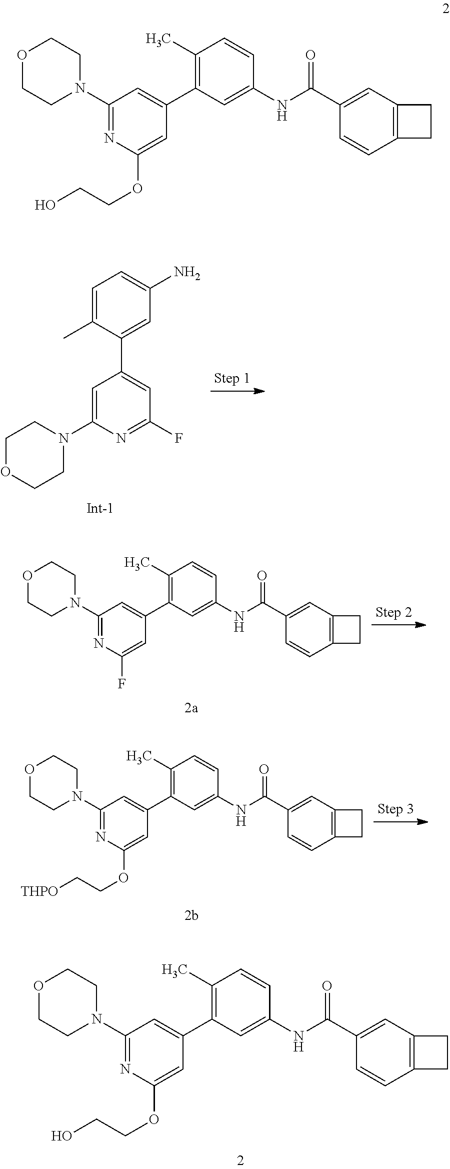

### Step 1

### N-(3-(2-fluoro-6-morpholinopyridin-4-yl)-4-methylphenyl)bicyclo[4.2.0]octa-1(6),2,4-triene-3-carboxamide 2a

The solution of Int-1 (100 mg, 348.03 μmol), bicyclo[4.2.0]octa-1,3,5-triene-3-carboxylic acid (57 mg, 382.83 μmol, Aldrich), HATU (199 mg, 522.05 μmol) and DIEA (135 mg, 1.04 mmol) in DMF (3 mL) was stirred at RT for 1 h. The mixture was diluted with water and extracted with EtOAc, the organic solution was dried and concentrated, the residue was purified by prep-TLC (EtOAc/hexane=1/1) to give target 2a (100 mg, 68.83% yield). MS m/z (ESI): 418.1 [M+H]⁺.

### Step 2

### N-(4-methyl-3-(2-morpholino-6-(2-((tetrahydro-2H-pyran-2-yl)oxy)ethoxy)pyridin-4-yl)phenyl)bicyclo[4.2.0]octa-1(6),2,4-triene-3-carboxamide 2b

To a solution of 2a (100 mg, 239.5 μmol) and 2-(tetrahydro-2H-pyran-2-yloxy)ethanol (210 mg, 1.43 mmol, Aldrich) in DMF (5 mL) was added NaH (58 mg, 2.39 mmol), the reaction was stirred at RT for 1 h and then at 80° C. for 16 h under Ar. The reaction was cooled and diluted with water, the mixture was extracted with EtOAc, and the organic solution was dried and concentrated. The residue was purified by prep-TLC (EtOAc/hexane=2/1) to give target 2b (30 mg, 65.3 μmol, 27.25% yield). MS m/z (ESI): 544.2 [M+H]⁺.

### Step 3

### N-(3-(2-(2-hydroxyethoxy)-6-morpholinopyridin-4-yl)-4-methylphenyl)bicyclo[4.2.0]octa-1(6),2,4-triene-3-carboxamide 2

To a solution of 2b (50 mg, 91.97 μmol) in CH₂Cl₂ (3 mL) was added TFA (53 mg, 459.85 μmol). The mixture was stirred at RT for 2 h. The mixture was concentrated, and pH was adjusted to 8 with the addition of aq. NaHCO₃, the mixture was extracted with EtOAc, the organic solution was dried and concentrated. The residue was purified by prep-HPLC to give target 2 (10 mg, 23.66% yield). 41 NMR (400 MHz, DMSO-d₆): δ 10.11 (s, 1H), 7.79 (d, 1H), 7.71 (dd, 1H), 7.66 (s, 2H), 7.23 (t, 2H), 6.24 (s, 1H), 6.03 (s, 1H), 4.26 (t, 1H), 3.73-3.69 (m, 6H), 3.47-3.44 (m, 4H), 3.20 (s, 4H), 2.21 (s, 3H). MS m/z (ESI): 460.6 [M+H]⁺.

### Example 3

### 3,3-difluoro-N-(3-(2-(2-hydroxyethoxy)-6-morpholinopyridin-4-yl)-4-methylphenyl)-2,3-dihydro-1H-indene-5-carboxamide 3

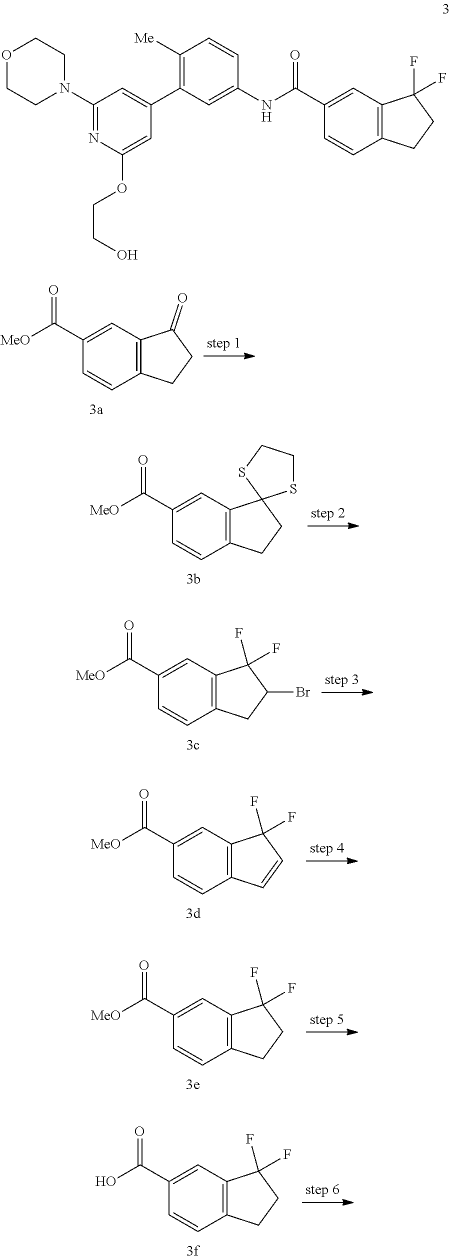

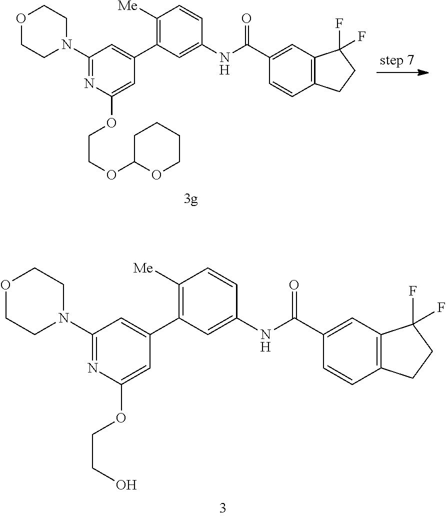 [SMILES: Cc1ccc(NC(=O)c2ccc3c(c2)C(F)(F)CC3)cc1-c1cc(OCCOC2CCCCO2)nc(N2CCOCC2)c1 ; Cc1ccc(NC(=O)c2ccc3c(c2)C(F)(F)CC3)cc1-c1cc(OCCO)nc(N2CCOCC2)c1]

### Step 1

### methyl 2′,3′-dihydrospiro[[1,3]dithiolane-2,1′-indene]-6′-carboxylate 3b

Boron trifluoride-diethyl ether (1.32 ml, 10.5 mmol) was added to a mixture of methyl 3-oxo-2,3-hydro-1H-indene-5-carboxylate 3a (500 mg, 2.6 mmol, Synthonix) and ethane-1,2-dithiol (0.33 mL, 4.0 mmol) in DCM (5.0 mL) at 0° C. The mixture was stirred overnight while the temperature was slowly warm to RT. The solution was slowly added in a 2N NaOH aqueous. The mixture was extracted with DCM (2×50 mL). The combined organic phase was washed with brine, dried over MgSO₄, filtered and concentrated by rotavapor. The residue was purified by silica gel chromatography using 0-30% DCM in hexane as eluent to afford the title compound 3b (640 mg, yield: 91%). MS m/z (ESI): 267 [M+H]⁺.

### Step 2

### methyl 2-bromo-3,3-difluoro-2,3-dihydro-1H-indene-5-carboxylate 3c

A 70% solution of HF in pyridine (2.75 mL, 106.0 mmol) was added to a suspension of 1,3-dibromo-5,5-dimethylimidazolidine-2,4-dione (2.48 g, 9.6 mmol) in DCM (25 mL) at −78° C. followed by stirring for 30 min. To it was added dropwise a solution of methyl 2,3-dihydrospiro[indene-1,2′-[1,3]dithiolane]-6-carboxylate 3b (0.64 g, 2.4 mmol) in DCM (5 mL) and stirred for 1 h at −78° C. and for 30 min at RT respectively. The reaction mixture was quenched with ice-cooled 2N NaOH (aq) and sat. Na2S₂O₃ (aq) and extracted with DCM. The organic phase was dried on MgSO₄, filtered and concentrated. The residue was purified by flash chromatography on a silica-gel column using 0-30% DCM in hexane as eluent to afford the title compound 3c (460 mg, yield: 66%). MS m/z (ESI): 292 [M+H]⁺.

### Step 3

### methyl 1,1-difluoro-1H-indene-6-carboxylate 3d

DBU (0.38 g, 2.47 mmol) was added to a solution of methyl 2-bromo-3,3-difluoro-2,3-dihydro-1H-indene-5-carboxylate 3c (0.48 g, 1.65 mmol) in DCM (7 mL) at 0° C. followed by stirring at RT for 18 h. The solution was purified by flash chromatography on a silica-gel column using 0-30% DCM in hexane as eluent to afford the title compound 3d (0.32 g, yield: 92%). MS m/z (ESI): 211 [M+H]⁺.

### Step 4

### methyl 3,3-difluoro-2,3-dihydro-1H-indene-5-carboxylate 3e

K₃PO₄ (42 mg, 0.20 mmol) and hydrazine hydrate (60 mg, 1.20 mmol) were slowly added to a solution of methyl 1,1-difluoro-1H-indene-6-carboxylate 3d (210 mg, 1.00 mmol) and 2-nitrobenzenesulfonyl chloride (222 mg, 1.00 mmol) in acetonitrile/DMSO (5/1 mL, v/v) at RT followed by stirring overnight. The mixture was diluted with EtOAc and washed with water. The organic phase was separated and dried over MgSO₄, filtered and concentrated. The residue was purified by flash chromatography on a silica-gel column using 0-25% DCM in hexane as eluent to afford the title compound 3e (125 mg, yield: 59%). MS m/z (ESI): 213 [M+H]⁺.

### Step 5

### 3,3-difluoro-2,3-dihydro-1H-indene-5-carboxylic acid 3f

A mixture of methyl 3,3-difluoro-2,3-dihydro-1H-indene-5-carboxylate 3e (62 mg, 0.29 mmol) and lithium hydroxide monohydrate (61 mg, 1.46 mmol) in MeOH/THF/H₂O (1:1:0.5 mL) was stirred at RT for 3 h. The mixture was concentrated and the residue was taken up in EtOAc and neutralized with 1.0 M HCl (aq). The aqueous phase was extracted with EtOAc. The combined organic phase was dried over MgSO₄, filtered, concentrated and dried in vacuum to afford the title compound 3f (21 mg, yield: 37%). MS m/z (ESI): 199 [M+H]⁺.

### Step 6

### 3,3-difluoro-N-(4-methyl-3-(2-morpholino-6-(2-((tetrahydro-2H-pyran-2-yl)oxy)ethoxy)pyridin-4-yl)phenyl)-2,3-dihydro-1H-indene-5-carboxamide 3g

iPrNEt (18 ul, 0.1 mmol) was added to a solution of 4-methyl-3-(2-morpholino-6-(2-((tetrahydro-2H-pyran-2-yl)oxy)ethoxy)pyridin-4-yl)aniline Int-2 (21 mg, 0.05 mmol), 3,3-difluoro-2,3-dihydro-1H-indene-5-carboxylic acid 3f (10 mg, 0.05 mmol) and HATU (24 mg, 0.06) in DMF (0.5 mL) at RT followed by stirring at RT for 3 h. The mixture was diluted with water, extracted with EtOAc. The organic phase was dried over MgSO₄, filtered, concentrated and dried in vacuum to afford the title compound 3g (26 mg, yield: 87%). MS m/z (ESI): 594 [M+H]⁺.

### Step 7

### 3,3-difluoro-N-(3-(2-(2-hydroxyethoxy)-6-morpholinopyridin-4-yl)-4-methylphenyl)-2,3-dihydro-1H-indene-5-carboxamide 3

A mixture of 3,3-difluoro-N-(4-methyl-3-(2-morpholino-6-(2-((tetrahydro-2H-pyran-2-yl)oxy)ethoxy)pyridin-4-yl)phenyl)-2,3-dihydro-1H-indene-5-carboxamide 3g (26 mg, 0.04 mmol) in 1.25 mol/L aq. HCl in MeOH (0.16 mL, 0.20 mmol) was stirred at RT for 1 h. The mixture was purified by HPLC with 10-60% MeCN in H₂O+0.1% TFA to afford the title compound 3 (8.8 mg, yield: 43.2%). ¹H NMR (400 MHz, Methanol-d₄) δ 8.02 (m, 2H), 7.96 (d, 1H), 7.50 (m, 1H), 7.39 (d, 1H), 7.17 (d, 1H), 6.18 (s, 1H), 6.06 (s, 1H), 4.27 (t, 2H), 3.78 (m, 2H), 3.70 (t, 4H), 3.42 (t, 4H), 3.02 (m, 2H), 2.54 (m, 2H), 2.16 (s, 3H). MS m/z (ESI): 510 [M+H]⁺.

### Example 4

### 3,3-difluoro-N-(3-(2-(2-hydroxyethoxy)-6-morpholinopyridin-4-yl)-4-methylphenyl)-2,3-dihydrobenzofuran-5-carboxamide 4

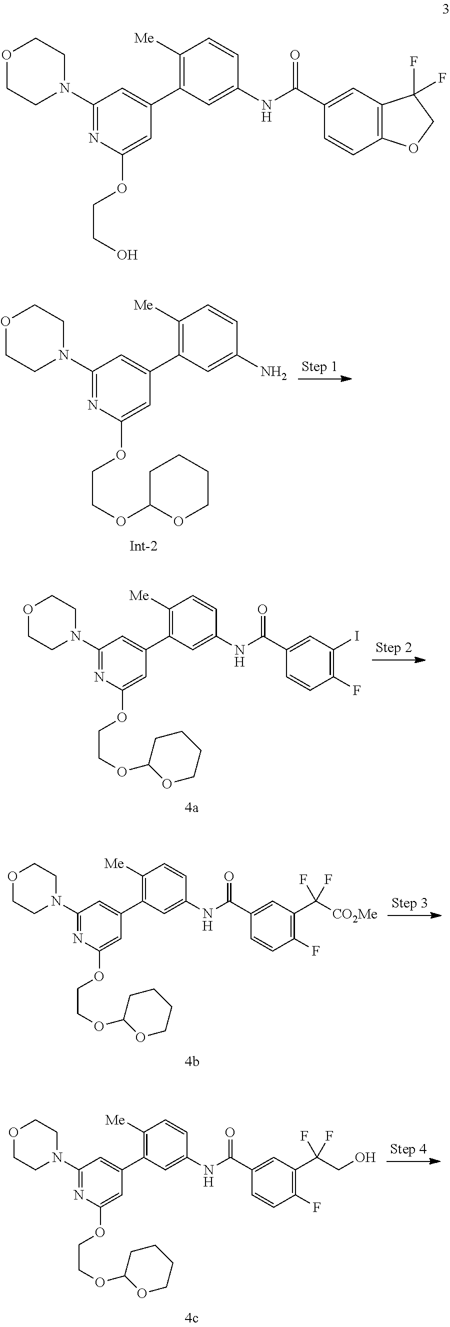 [SMILES: Cc1ccc(NC(=O)c2ccc3c(c2)C(F)(F)CO3)cc1-c1cc(OCCO)nc(N2CCOCC2)c1 ; Cc1ccc(N)cc1-c1cc(OCCOC2CCCCO2)nc(N2CCOCC2)c1 ; Cc1ccc(NC(=O)c2ccc(F)c(I)c2)cc1-c1cc(OCCOC2CCCCO2)nc(N2CCOCC2)c1 ; COC(=O)C(F)(F)c1cc(C(=O)Nc2ccc(C)c(-c3cc(OCCOC4CCCCO4)nc(N4CCOCC4)c3)c2)ccc1F ; Cc1ccc(NC(=O)c2ccc(F)c(C(F)(F)CO)c2)cc1-c1cc(OCCOC2CCCCO2)nc(N2CCOCC2)c1]

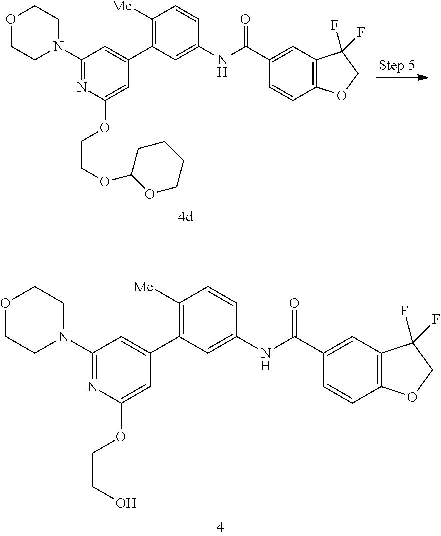 [SMILES: Cc1ccc(NC(=O)c2ccc3c(c2)C(F)(F)CO3)cc1-c1cc(OCCOC2CCCCO2)nc(N2CCOCC2)c1 ; Cc1ccc(NC(=O)c2ccc3c(c2)C(F)(F)CO3)cc1-c1cc(OCCO)nc(N2CCOCC2)c1]

### Step 1

### 4-fluoro-3-iodo-N-(4-methyl-3-(2-morpholino-6-(2-((tetrahydro-2H-pyran-2-yl)oxy)ethoxy)pyridin-4-yl)phenyl)benzamide 4a

iPrNEt (0.10 ml, 0.48 mmol) was added to a solution of 4-methyl-3-(2-morpholino-6-(2-((tetrahydro-2H-pyran-2-yl)oxy)ethoxy)pyridin-4-yl)aniline Int-2 (160 mg, 0.39 mmol), 4-fluoro-3-iodobenzoic acid (103 mg, 0.39 mmol, Combi-Blocks) and HATU (185 mg, 0.48) in DMF (2.5 mL) at RT followed by stirring at RT for 3 h. The mixture was diluted with water, extracted with EtOAc. The organic phase was dried over MgSO₄, filtered, concentrated. The residue was purified by flash chromatography on a silica-gel column using 0-75% EtOAc in DCM as eluent to afford the title compound 4a (110 mg, yield: 43%). MS m/z (ESI): 662 [M+H]⁺.

### Step 2

### methyl 2,2-difluoro-2-(2-fluoro-5-((4-methyl-3-(2-morpholino-6-(2-((tetrahydro-2H-pyran-2-yl)oxy)ethoxy)pyridin-4-yl)phenyl)carbamoyl)phenyl)acetate 4b

Cu powder (33 mg, 0.51 mmol) was added to a solution of 4-fluoro-3-iodo-N-(4-methyl-3-(2-morpholino-6-(2-((tetrahydro-2H-pyran-2-yl)oxy)ethoxy)pyridin-4-yl)phenyl)benzamide 4a (110 mg, 0.17 mmol) in DMSO (2.0 mL) at RT. The mixture was stirred for 1 h. To it was added methyl 2-bromo-2,2-difluoroacetate (104 mg, 0.51 mmol) followed by stirring at 80° C. for 18 h. After cooling, diluted with EtOAc, washed with water. The aqueous was extracted with EtOAc. The organic phase was dried over MgSO₄, filtered, concentrated. The residue was purified by flash chromatography on a silica-gel column using 0-80% EtOAc in DCM as eluent to afford the title compound 4b (102 mg, yield: 96%). MS m/z (ESI): 645 [M+H]⁺.

### Step 3

### 3-(1,1-difluoro-2-hydroxyethyl)-4-fluoro-N-(4-methyl-3-(2-morpholino-6-(2-((tetrahydro-2H-pyran-2-yl)oxy)ethoxy)pyridin-4-yl)phenyl)benzamide 4c

Sodium borohydride (17 mg, 0.45 mmol) was added to a solution of methyl 2,2-difluoro-2-(2-fluoro-5-((4-methyl-3-(2-morpholino-6-(2-((tetrahydro-2H-pyran-2-yl)oxy)ethoxy)pyridin-4-yl)phenyl)carbamoyl)phenyl)acetate 4b (100 mg, 0.15 mmol) in THF/H₂O (2.0:0.05 mL) at −78° C. The mixture was warmed to RT and stirred for 5 h. To it was added ice-water and the mixture was extracted with EtOAc. The organic phase was dried over MgSO₄, filtered, concentrated. The residue was purified by flash chromatography on a silica-gel column using 0-10% MeOH in DCM as eluent to afford the title compound 4c (25 mg, yield: 27%). MS m/z (ESI): 616 [M+H]⁺.

### Step 4

### 3,3-difluoro-N-(4-methyl-3-(2-morpholino-6-(2-((tetrahydro-2H-pyran-2-yl)oxy)ethoxy)pyridin-4-yl)phenyl)-2,3-dihydrobenzofuran-5-carboxamide 4d

A mixture of 3-(1,1-difluoro-2-hydroxyethyl)-4-fluoro-N-(4-methyl-3-(2-morpholino-6-(2-((tetrahydro-2H-pyran-2-yl)oxy)ethoxy)pyridin-4-yl)phenyl)benzamide 4c (25 mg, 0.04 mmol), 18-crown-6 (5 mg, 0.02 mmol) and Cs₂CO₃ (39 mg, 0.12 mmol) in THF (2 mL) was stirred at 80° C. for 18 h. After cooling, the mixture was purified by HPLC using 10-75% acetonitrile in H₂O+0.1% TFA to afford the title compound 4d (12 mg, yield: 50%). MS m/z (ESI): 596 [M+H]⁺.

### Step 5

### 3,3-difluoro-N-(3-(2-(2-hydroxyethoxy)-6-morpholinopyridin-4-yl)-4-methylphenyl)-2,3-dihydrobenzofuran-5-carboxamide 4

A mixture of 3,3-difluoro-N-(4-methyl-3-(2-morpholino-6-(2-((tetrahydro-2H-pyran-2-yl)oxy)ethoxy)pyridin-4-yl)phenyl)-2,3-dihydrobenzofuran-5-carboxamide 4d (12 mg, 0.02 mmol) in 1.25 M HCl in MeOH (0.08 ml, 0.10 mmol) was stirred at RT for 1 h. The mixture was purified by HPLC with 10-60% MeCN in H₂O+0.1% TFA to afford the title compound 4. MS m/z (ESI): 512 [M+H]⁺.

### Example 5

### 1,1-difluoro-N-(3-(2-(2-hydroxyethoxy)-6-morpholinopyridin-4-yl)-4-methylphenyl)-2,3-dihydro-1H-indene-5-carboxamide 5

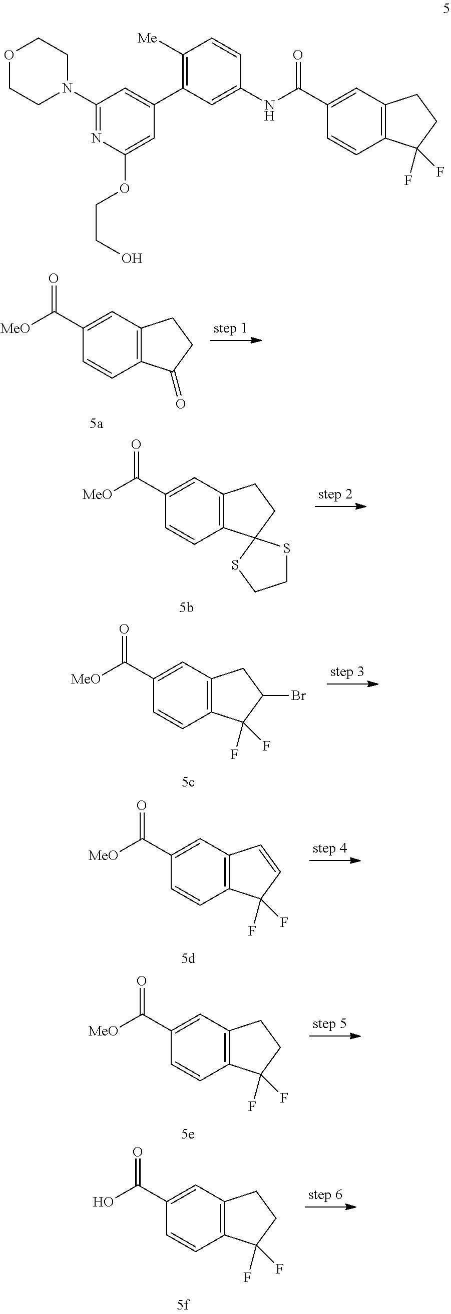

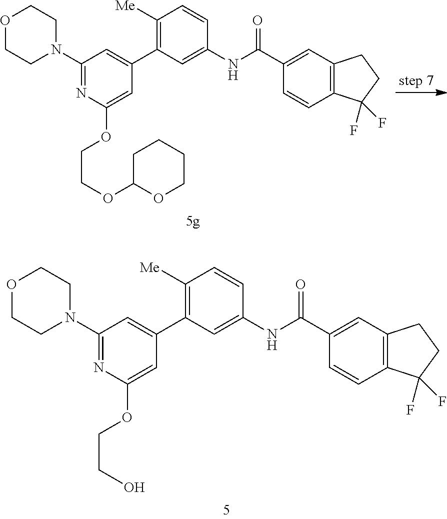 [SMILES: Cc1ccc(NC(=O)c2ccc3c(c2)CCC3(F)F)cc1-c1cc(OCCOC2CCCCO2)nc(N2CCOCC2)c1 ; Cc1ccc(NC(=O)c2ccc3c(c2)CCC3(F)F)cc1-c1cc(OCCO)nc(N2CCOCC2)c1]

### Step 1

### methyl 2,3-dihydrospiro[indene-1,2′-[1,3]dithiolane]-5-carboxylate 5b

The title compound was prepared from the reaction between methyl 1-oxo-2,3-dihydro-1H-indene-5-carboxylate 5a (AstaTech) and ethane-1,2-dithiol by following Step 1 experimental procedure for Example 3. MS m/z (ESI): 267 [M+H]⁺.

### Step 2

### methyl 2-bromo-1,1-difluoro-2,3-dihydro-1H-indene-5-carboxylate 5c

The title compound was prepared from the reaction between methyl 2,3-dihydrospiro[indene-1,2′-[1,3]dithiolane]-5-carboxylate 5b, 1,3-dibromo-5,5-dimethylimidazolidine-2,4-dione and 70% solution of HF in pyridine by following Step 2 experimental procedure for Example 3. MS m/z (ESI): 292 [M+H]⁺.

### Step 3

### methyl 1,1-difluoro-1H-indene-5-carboxylate 5d

The title compound was prepared from the reaction between methyl 2-bromo-1,1-difluoro-2,3-dihydro-1H-indene-5-carboxylate 5c and DBU by following Step 3 experimental procedure for Example 3. MS m/z (ESI): 211 [M+H]⁺.

### Step 4

### methyl 1,1-difluoro-2,3-dihydro-1H-indene-5-carboxylate 5e

The title compound was prepared from the reaction between methyl 1,1-difluoro-1H-indene-5-carboxylate 5d, hydrazine hydrate and 2-nitrobenzenesulfonyl chloride by following Step 4 experimental procedure for Example 3. MS m/z (ESI): 213 [M+H]⁺.

### Step 5

### 1,1-difluoro-2,3-dihydro-1H-indene-5-carboxylic acid 5f

The title compound was prepared from the reaction between methyl 1,1-difluoro-2,3-dihydro-1H-indene-5-carboxylate 5e and lithium hydroxide monohydrate by following Step 5 experimental procedure for Example 3. MS m/z (ESI): 199 [M+H]⁺.

### Step 6

### 1,1-difluoro-N-(4-methyl-3-(2-morpholino-6-(2-((tetrahydro-2H-pyran-2-yl)oxy)ethoxy)pyridin-4-yl)phenyl)-2,3-dihydro-1H-indene-5-carboxamide 5g

The title compound was prepared from the reaction between 4-methyl-3-(2-morpholino-6-(2-((tetrahydro-2H-pyran-2-yl)oxy)ethoxy)pyridin-4-yl)aniline Int-2 and 3,3-difluoro-2,3-dihydro-1H-indene-5-carboxylic acid 5f by following Step 6 experimental procedure for Example 3. MS m/z (ESI): 594 [M+H]⁺.

### Step 7

### 1,1-difluoro-N-(3-(2-(2-hydroxyethoxy)-6-morpholinopyridin-4-yl)-4-methylphenyl)-2,3-dihydro-1H-indene-5-carboxamide 5

The title compound was prepared from the reaction 1,1-difluoro-N-(4-methyl-3-(2-morpholino-6-(2-((tetrahydro-2H-pyran-2-yl)oxy)ethoxy)pyridin-4-yl)phenyl)-2,3-dihydro-1H-indene-5-carboxamide 5g and 1.25 mmol/L aq. HCl in MeOH by following Step 7 experimental procedure for Example 3. ¹H NMR (400 MHz, Methanol-d₄) δ 7.83 (m, 2H), 7.52 (m, 3H), 7.20 (d, 1H), 6.17 (m, 1H), 6.03 (m, 1H), 4.30 (t, 2H), 3.80 (m, 2H), 3.73 (t, 4H), 3.43 (t, 4H), 3.06 (m, 2H), 2.57 (m, 2H), 2.19 (s, 3H). MS m/z (ESI): 510 [M+H]⁺.

### Example 6

### 7,7-difluoro-N-(3-(2-(2-hydroxyethoxy)-6-morpholinopyridin-4-yl)-4-methylphenyl)-6,7-dihydro-5H-cyclopenta[b]pyridine-3-carboxamide 6

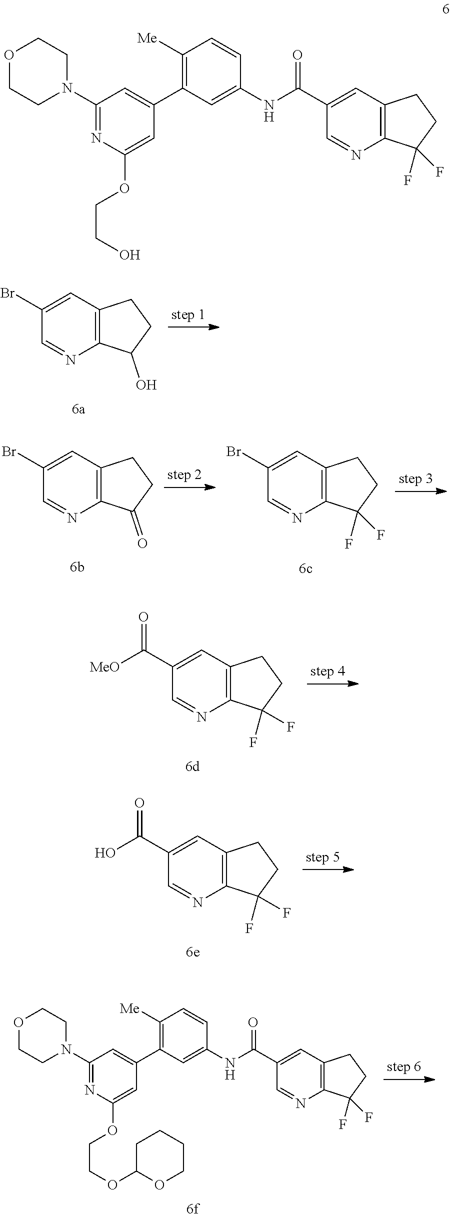

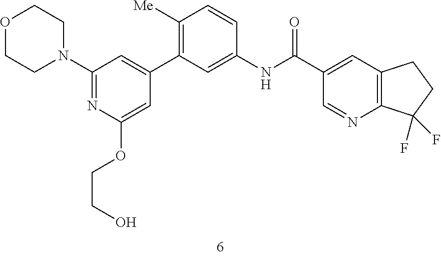 [SMILES: Cc1ccc(NC(=O)c2cnc3c(c2)CCC3(F)F)cc1-c1cc(OCCO)nc(N2CCOCC2)c1]

### Step 1

### 3-bromo-5,6-dihydro-7H-cyclopenta[b]pyridin-7-one 6b

Dess-Martin periodinane (1.85 g, 4.4 mmol) was added partially to a solution of 3-bromo-6,7-dihydro-5H-cyclopenta[b]pyridin-7-ol 6a (0.75 g, 3.5 mmol) in DCM at 0° C. The mixture was stirred for 3 h while the temperature was slowly warm to RT. The mixture was quenched with 1N NaOH aq. and extracted with DCM (2×50 mL). The combined organic phase was washed with brine, dried over MgSO₄, filtered and concentrated by rotavapor. The residue was purified by silica gel chromatography using 0-55% EtOAc in hexane as eluent to afford the title compound 6b (0.74 g, yield: 68%). MS m/z (ESI): 212 [M+H]⁺.

### Step 2

### 3-bromo-7,7-difluoro-6,7-dihydro-5H-cyclopenta[b]pyridine 6c

Deoxo-Fluor (563 mg, 2.54 mmol) was added dropwise to a solution of 3-bromo-5,6-dihydro-7H-cyclopenta[b]pyridin-7-one 6b (270 mg, 1.27 mmol) in DCM (5 mL) at 0° C., followed by stirring at RT for 3 days. The reaction mixture was purified by flash chromatography on a silica-gel column using 0-60% EtOAc in hexane as eluent to afford the title compound 6c (72 mg, yield: 24%). MS m/z (ESI): 235 [M+H]⁺.

### Step 3

### methyl 7,7-difluoro-6,7-dihydro-5H-cyclopenta[b]pyridine-3-carboxylate 6d

A vial was charged with 3-bromo-7,7-difluoro-6,7-dihydro-5H-cyclopenta[b]pyridine 6c (60 mg, 0.26 mmol) and Pd(dppf)Cl₂ (19 mg, 0.026 mmol) and capped. The mixture was degassed by vacuum/refilling CO(g). Then MeOH (1.0 ml) and Et₃N (0.072 ml, 0.52 mmol) was added. The reaction was run at 65° C. under CO balloon. After cooling, the mixture was concentrated and the residue was purified by flash chromatography on a silica-gel column using 0-60% EtOAc in hexane as eluent to afford the title compound 6d (24 mg, yield: 47%). MS m/z (ESI): 214 [M+H]⁺.

### Step 4

### 7,7-difluoro-6,7-dihydro-5H-cyclopenta[b]pyridine-3-carboxylic acid 6e

The title compound was prepared from the reaction between methyl 7,7-difluoro-6,7-dihydro-5H-cyclopenta[b]pyridine-3-carboxylate 6d and lithium hydroxide monohydrate by following Step 5 experimental procedure for Example 3. MS m/z (ESI): 200 [M+H]⁺.

### Step 5

### 7,7-difluoro-N-(4-methyl-3-(2-morpholino-6-(2-((tetrahydro-2H-pyran-2-yl)oxy)ethoxy)pyridin-4-yl)phenyl)-6,7-dihydro-5H-cyclopenta[b]pyridine-3-carboxamide 6f

The title compound was prepared from the reaction between 4-methyl-3-(2-morpholino-6-(2-((tetrahydro-2H-pyran-2-yl)oxy)ethoxy)pyridin-4-yl)aniline Int-2 and 7,7-difluoro-6,7-dihydro-5H-cyclopenta[b]pyridine-3-carboxylic acid 6e by following Step 6 experimental procedure for Example 3. MS m/z (ESI): 595 [M+H]⁺.

### Step 6

### 7,7-difluoro-N-(3-(2-(2-hydroxyethoxy)-6-morpholinopyridin-4-yl)-4-methylphenyl)-6,7-dihydro-5H-cyclopenta[b]pyridine-3-carboxamide 6

The title compound was prepared from the reaction 7,7-difluoro-N-(4-methyl-3-(2-morpholino-6-(2-((tetrahydro-2H-pyran-2-yl)oxy)ethoxy)pyridin-4-yl)phenyl)-6,7-dihydro-5H-cyclopenta[b]pyridine-3-carboxamide 6f and 1.25 mmol/L aq. HCl in MeOH by following Step 7 experimental procedure for Example 3. ¹H NMR (400 MHz, Methanol-d₄) δ 9.06 (s, 1H), 8.34 (s, 1H), 7.60 (m, 2H), 7.27 (d, 1H), 6.23 (s, 1H), 6.11 (s, 1H), 4.38 (t, 2H), 3.89 (m, 2H), 3.80 (t, 4H), 3.51 (t, 4H), 3.15 (m, 2H), 2.72 (m, 2H), 2.26 (s, 3H). MS m/z (ESI): 511 [M+H]⁺.

### Example 7

### 5,5-difluoro-N-(3-(2-(2-hydroxyethoxy)-6-morpholinopyridin-4-yl)-4-methylphenyl)-6,7-dihydro-5H-cyclopenta[b]pyridine-3-carboxamide 7

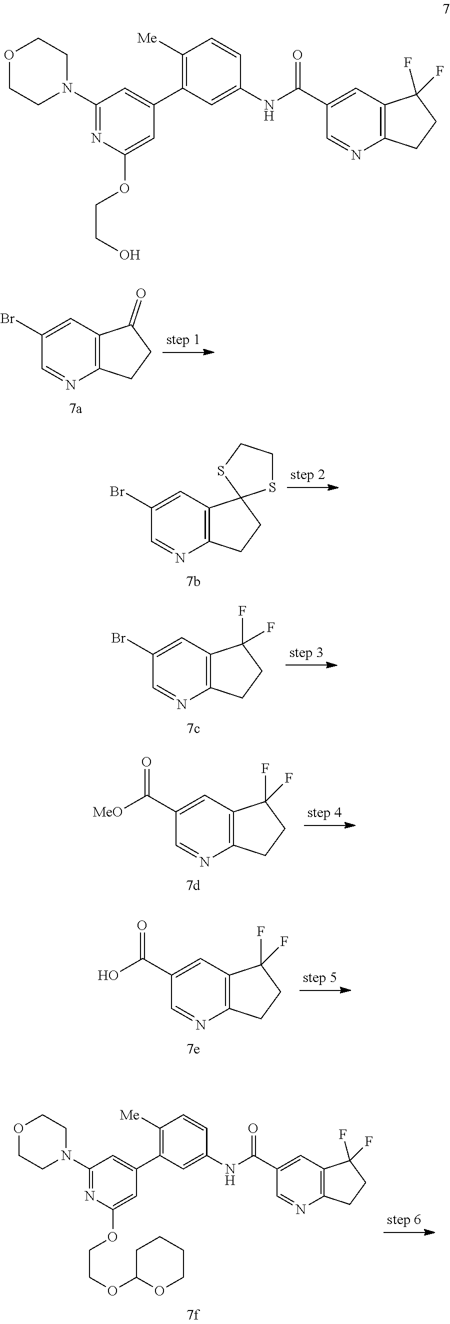

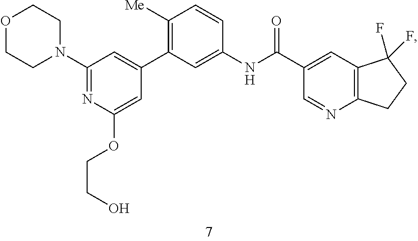 [SMILES: Cc1ccc(NC(=O)c2cnc3c(c2)C(F)(F)CC3)cc1-c1cc(OCCO)nc(N2CCOCC2)c1]

### Step 1

### 3-bromo-6,7-dihydrospiro[cyclopenta[b]pyridine-5,2′-[1,3]dithiolane] 7b

The title compound was prepared from the reaction between 3-bromo-6,7-dihydro-5H-cyclopenta[b]pyridin-5-one and ethane-1,2-dithiol by following Step 1 experimental procedure for Example 3. MS m/z (ESI): 289 [M+H]⁺.

### Step 2

### 3-bromo-5,5-difluoro-6,7-dihydro-5H-cyclopenta[b]pyridine 7c

The title compound was prepared from the reaction between 3-bromo-6,7-dihydrospiro[cyclopenta[b]pyridine-5,2′-[1,3]dithiolane] 7b, 1,3-dibromo-5,5-dimethylimidazolidine-2,4-dione and 70% solution of HF in pyridine by following Step 2 experimental procedure for Example 3. MS m/z (ESI): 235 [M+H]⁺.

### Step 3

### methyl 5,5-difluoro-6,7-dihydro-5H-cyclopenta[b]pyridine-3-carboxylate 7d

The title compound was prepared from the reaction between 3-bromo-5,5-difluoro-6,7-dihydro-5H-cyclopenta[b]pyridine 7c, MeOH and CO (g) by following Step 3 experimental procedure for Example 6. MS m/z (ESI): 214 [M+H]⁺.

### Step 4

### 5,5-difluoro-6,7-dihydro-5H-cyclopenta[b]pyridine-3-carboxylic acid 7e

The title compound was prepared from the reaction between methyl 5,5-difluoro-6,7-dihydro-5H-cyclopenta[b]pyridine-3-carboxylate 7d and lithium hydroxide monohydrate by following Step 5 experimental procedure for Example 3. MS m/z (ESI): 200 [M+H]⁺.

### Step 5

### 5,5-difluoro-N-(4-methyl-3-(2-morpholino-6-(2-((tetrahydro-2H-pyran-2-yl)oxy)ethoxy)pyridin-4-yl)phenyl)-6,7-dihydro-5H-cyclopenta[b]pyridine-3-carboxamide 7f

The title compound was prepared from the reaction between 4-methyl-3-(2-morpholino-6-(2-((tetrahydro-2H-pyran-2-yl)oxy)ethoxy)pyridin-4-yl)aniline Int-2 and 5,5-difluoro-6,7-dihydro-5H-cyclopenta[b]pyridine-3-carboxylic acid 7e by following Step 6 experimental procedure for Example 3. MS m/z (ESI): 595 [M+H]⁺.

### Step 6

### 5,5-difluoro-N-(3-(2-(2-hydroxyethoxy)-6-morpholinopyridin-4-yl)-4-methylphenyl)-6,7-dihydro-5H-cyclopenta[b]pyridine-3-carboxamide 7

The title compound was prepared from the reaction 5,5-difluoro-N-(4-methyl-3-(2-morpholino-6-(2-((tetrahydro-2H-pyran-2-yl)oxy)ethoxy)pyridin-4-yl)phenyl)-6,7-dihydro-5H-cyclopenta[b]pyridine-3-carboxamide 7f and 1.25 mmol/L aq. HCl in MeOH by following Step 7 experimental procedure for Example 3. ¹H NMR (400 MHz, Methanol-d₄) δ 9.22 (s, 1H), 8.54 (d, J=2.0 Hz, 1H), 7.63 (m, 2H), 7.31 (d, 1H), 6.33 (s, 1H), 6.20 (s, 1H), 4.40 (t, 2H), 3.90 (m, 2H), 3.82 (t, 4H), 3.55 (t, 4H), 3.27 (m, 2H), 2.77 (m, 2H), 2.29 (s, 3H). MS m/z (ESI): 511 [M+H]⁺.

### Example 8

### N-(3-(2-(2-hydroxyethoxy)-6-morpholinopyridin-4-yl)-4-methylphenyl)-2,2-dimethylbenzo[d][1,3]dioxole-5-carboxamide 8

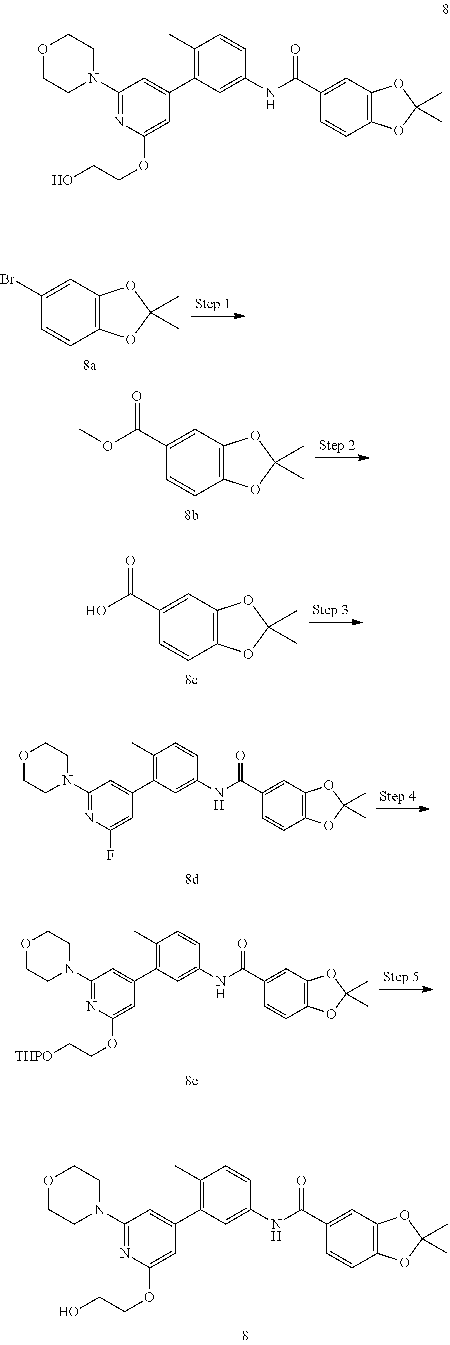

### Step 1

### Methyl 2,2-dimethylbenzo[d][1,3]dioxole-5-carboxylate 8b

To a solution of 5-bromo-2,2-dimethylbenzo[d][1,3]dioxole 8a (300 mg, 1.31 mmol, Bidepharm) and (C₂H₅)₃N (1.33 g, 13.10 mmol) in DMSO (2 mL) was added Pd(dppf)Cl₂ (191.47 mg, 261.93 μmol), the reaction was filled with CO(g) and stirred at 120° C. for 16 h. The mixture was diluted with EtOAc and washed with brine, the organic solution was concentrated, and the residue was purified by prep-TLC (EtOAc/hexane=1/10) to give target 8b (100 mg, 480.28 μmol, 36.67% yield). LCMS: MS m/z (ESI): 209.1 [M+H]⁺.

### Step 2

### 2,2-dimethylbenzo[d][1,3]dioxole-5-carboxylic acid 8c

To a solution of 8b (100 mg, 480.28 μmol) in H₂O (2 mL) and THF (2 mL) was added LiOH (115.03 mg, 4.80 mmol), the mixture was stirred at RT for 16 h. The pH was adjusted to 5 and the mixture was extracted with EtOAc, the organic solution was concentrated to give crude target 8c (50 mg, 257.49 μmol, 53.61% yield). LCMS: MS m/z (ESI): 195.4 [M+H]⁺.

### Step 3

### N-(3-(2-fluoro-6-morpholinopyridin-4-yl)-4-methylphenyl)-2,2-dimethylbenzo[d][1,3]dioxole-5-carboxamide 8d

To a solution of Int-1 (100.00 mg, 348.03 μmol) and 8c (67.58 mg, 348.03 μmol) in DMF (3 mL) was added DIEA (134.69 mg, 1.04 mmol) and HATU (158.80 mg, 417.64 μmol). The mixture was stirred at RT for 1 h. The mixture was diluted with EtOAc and washed with brine. The organic solution was concentrated, and the residue was purified by prep-TLC (MeOH/DCM=1/10) to give target 8d (80 mg, 172.60 μmol, 49.59% yield). LCMS: MS m/z (ESI): 464.0 [M+H]⁺.

### Step 4

### 2,2-dimethyl-N-(4-methyl-3-(2-morpholino-6-(2-((tetrahydro-2H-pyran-2-yl)oxy)ethoxy)pyridin-4-yl)phenyl)benzo[d][1,3]dioxole-5-carboxamide 8e

To a solution of 8d (80 mg, 172.60 μmol) and 2-(tetrahydro-2H-pyran-2-yloxy)ethanol (126.16 mg, 863.00 μmol) in dry DMF (3 mL) was added NaH (34.52 mg, 863 μmol, 60% wt), then the mixture was stirred at RT for 30 min under Ar and at 80° C. for 3 h. The mixture was cooled and quenched with water, then the mixture was extracted with EA, the organic solution was dried and concentrated, the residue was purified by prep-TLC (MeOH/DCM=1/8) to give target 8e (60 mg, 101.75 μmol, 58.95% yield). LCMS: MS m/z (ESI): 590.1 [M+H]⁺.

### Step 5

### N-(3-(2-(2-hydroxyethoxy)-6-morpholinopyridin-4-yl)-4-methylphenyl)-2,2-dimethylbenzo[d][1,3]dioxole-5-carboxamide 8

To a solution of 8e (88.05 mg, 149.32 μmol) in DCM (2 mL) was added TFA (170.26 mg, 1.49 mmol), the mixture was stirred at RT for 1 h. The mixture was concentrated, and the residue was purified by prep-HPLC to give target 8 (15 mg, 29.67 μmol, 19.87% yield). ¹H NMR (400 MHz, DMSO-d₆): δ 10.02 (s, 1H), 7.69 (d, 1H), 7.62 (d, 1H), 7.51 (d, 9.2 Hz, 1H), 7.41 (s, 1H), 7.24 (d, 1H), 6.96 (d, 8.4 Hz, 1H), 6.24 (s, 1H), 6.02 (s, 1H), 4.82 (t, 5.2 Hz, 1H), 4.25 (t, 2H), 3.72-3.68 (m, 6H), 3.54-3.49 (m, 4H), 2.21 (s, 3H), 1.68 (s, 6H). LCMS: MS m/z (ESI): 506.1 [M+H]⁺.

### Example 9

### N-(3-(2-(2-hydroxyethoxy)-6-morpholinopyridin-4-yl)-4-methylphenyl)benzo[d][1,3]dioxole-5-carboxamide 9

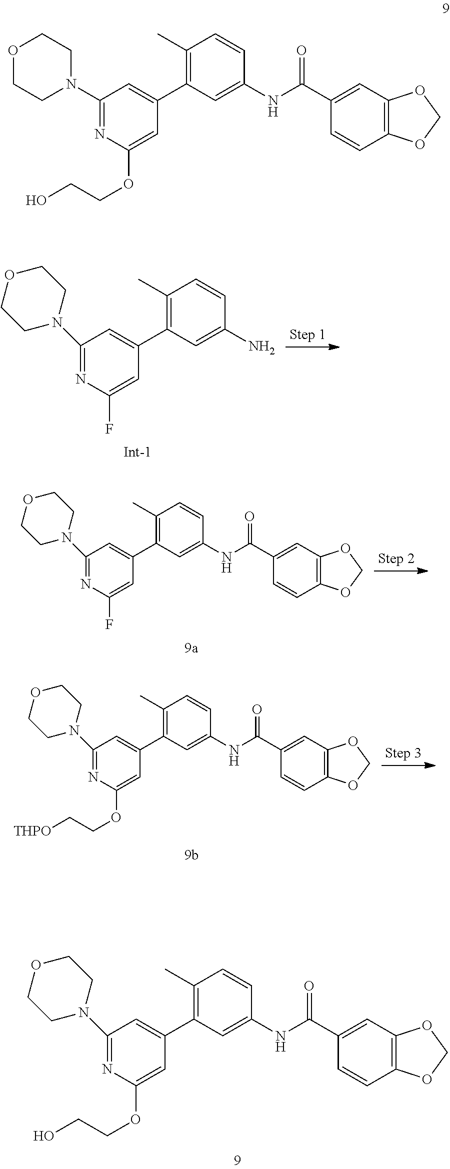

### Step 1

### N-(3-(2-fluoro-6-morpholinopyridin-4-yl)-4-methylphenyl)benzo[d][1,3]dioxole-5-carboxamide 9a

To a solution of Int-1 (100 mg, 348.03 μmol), 3,4-(methylenedioxy)benzoic acid (63.60 mg, 382.83 μmol) and DIEA (134.94 mg, 1.04 mmol) in DMF (1 mL) was added HATU (145.56 mg, 382.83 μmol) at room temperature. Then the resulting solution was stirred at room temperature for 4 hours. Water (50 mL) was added and the mixture was extracted with EtOAc (50 mL×3). The organic phase was combined, dried over anhydrous Na₂SO₄, filtered and concentrated. The residue was purified by silica-gel column chromatography (hexane:EtOAc=10:1) to afford target product 9a (120 mg, 275.58 μmol, 79.18% yield). LCMS: MS m/z (ESI): 436.1 [M+H]⁺.

### Step 2

### N-(4-methyl-3-(2-morpholino-6-(2-((tetrahydro-2H-pyran-2-yl)oxy)ethoxy)pyridin-4-yl)phenyl)benzo[d][1,3]dioxole-5-carboxamide 9b

To a solution of 9a (110 mg, 252.61 μmol) in DMF (2 mL) was added 2-(tetrahydro-2H-pyran-2-yloxy)ethanol (184.64 mg, 1.26 mmol, Aldrich) to give a yellow solution. NaH (12.13 mg, 505.23 μmol) was added carefully and the reaction was stirred at RT for 30 min, and then the mixture was warmed to 60° C. overnight. The reaction mixture was cool to room temperature, quenched with aqueous sodium bicarbonate (50 mL), extracted with EtOAc (50 mL×3). The organic phase was combined, dried over anhydrous Na₂SO₄, filtered and concentrated. The residue was purified by silica-gel column chromatography (hexane: EtOAc=10:1) to afford target product 9b (85 mg, 151.35 μmol, 59.91% yield). LCMS: MS m/z (ESI): 562.2 [M+H]⁺.

### Step 3

### N-(3-(2-(2-hydroxyethoxy)-6-morpholinopyridin-4-yl)-4-methylphenyl)benzo[d][1,3]dioxole-5-carboxamide 9

To a solution of 9b (85 mg, 151.35 μmol) in DCM (1 mL) was added TFA (1 mL) at room temperature. Then the resulting solution was stirred at RT for 2 hours. The mixture was diluted with saturated sodium bicarbonate (50 mL), extracted with EtOAc (50 mL×3). The organic phase was combined, dried over anhydrous Na₂SO₄, filtered and concentrated. The residue was purified by prep-HPLC to afford target product 9 (35 mg, 73.30 μmol, 48.43% yield). ¹H NMR (400 MHz, DMSO-d₆): δ 10.06 (s, 1H), 7.70 (dd, 1H), 7.63 (d, 1H), 7.57 (dd, 1H), 7.51 (d, 1H), 7.24 (d, 1H), 7.06 (d, 1H), 6.25 (s, 1H), 6.13 (s, 2H), 6.03 (s, 1H), 4.54 (br, 5H), 4.26 (t, 2H), 3.73-3.68 (m, 6H), 2.21 (s, 3H). LCMS: MS m/z (ESI): 478.1 [M+H]⁺.

### Example 10

### 2,2-difluoro-N-(3-(2-(2-hydroxyethoxy)-6-morpholinopyridin-4-yl)-4-methylphenyl)-[1,3]dioxolo[4,5-c]pyridine-6-carboxamide 10

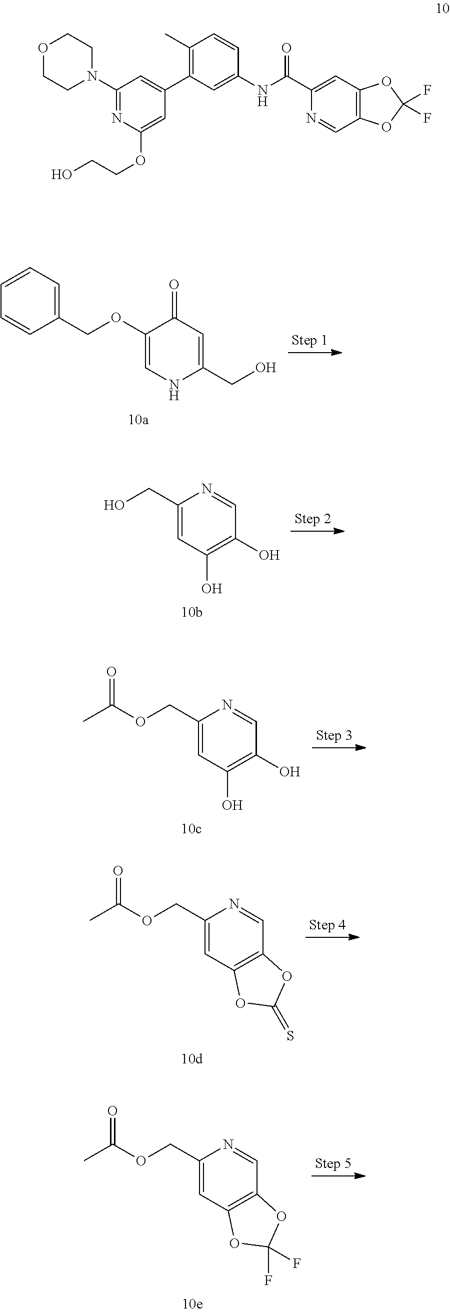 [SMILES: Cc1ccc(NC(=O)c2cc3c(cn2)OC(F)(F)O3)cc1-c1cc(OCCO)nc(N2CCOCC2)c1 ; O=c1cc(CO)[nH]cc1OCc1ccccc1 ; OCc1cc(O)c(O)cn1 ; CC(=O)OCc1cc(O)c(O)cn1 ; CC(=O)OCc1cc2oc(=S)oc2cn1 ; CC(=O)OCc1cc2c(cn1)OC(F)(F)O2]

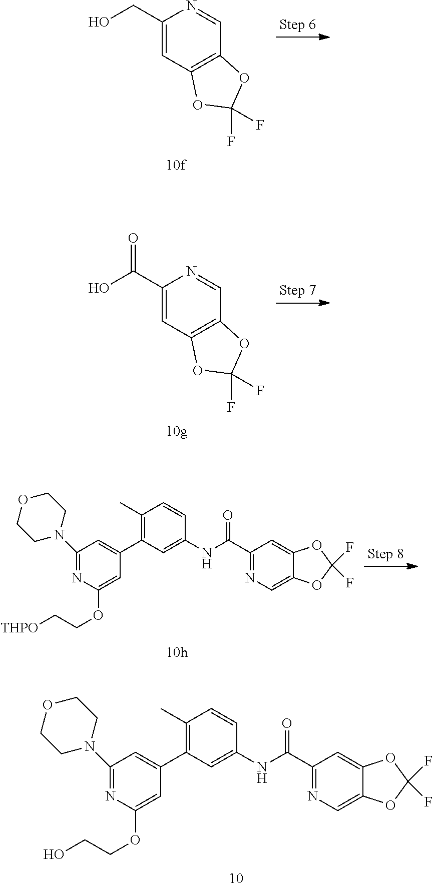

### Step 1

### 6-(hydroxymethyl)pyridine-3,4-diol 10b

A solution of 5-(benzyloxy)-2-(hydroxymethyl)pyridin-4(1H)-one 10a (2.500 g, 10.81 mmol, Bidepharm) in MeOH (20 mL) was degassed with H₂ three times, then the reaction was stirred at RT under H₂ for 1 h. The mixture was filtered and the filtrate was concentrated to give target 10b (1.000 g, 7.09 mmol, 65.54% yield). LCMS: MS m/z (ESI): 142.2 [M+H]⁺.

### Step 2

### (4,5-dihydroxypyridin-2-yl)methyl acetate 10c

A solution of 10b (1.00 g, 7.09 mmol) in HOAc (21 mL) was stirred at 120° C. for 16 h. The mixture was concentrated to give crude target 10c (1.00 g, 5.46 mmol, 77.05% yield) for next step. LCMS: MS m/z (ESI): 184.0 [M+H]⁺.

### Step 3

### (2-thioxo-[1,3]dioxolo[4,5-c]pyridin-6-yl)methyl acetate 10d

Thiocarbonyl dichloride (376.66 mg, 3.28 mmol, Energy) was added slowly to a stirred suspension of 10c (400 mg, 2.18 mmol) and DMAP (533.61 mg, 4.37 mmol) in DCM at 0° C. under N₂. During the addition, the formation of a light red precipitate was immediately observed. The reaction was allowed to warm to RT and stirred for 2 h, then the solution was diluted with water. The organic layer was separated and the aqueous layer was extracted with DCM (100 mL×3). The combined organic layers were washed with brine, dried over MgSO₄, filtered and concentrated. The residue was purified by silica gel chromatography (EtOAc/hexane=1/10) to give the product 10d (300 mg, 1.30 mmol, 59.43% yield). LCMS: MS m/z (ESI): 226.1 [M+H]⁺.

### Step 4

### (2,2-difluoro-[1,3]dioxolo[4,5-c]pyridin-6-yl)methyl acetate 10e

HF-pyridine (737.54 mg, 9.32 mmol) was added dropwise to a stirred solution of 10d (300 mg, 1.33 mmol) in DCM (6 mL) under nitrogen at −78° C. in a polypropylene vessel by using a plastic syringe. Then 1,3-dibromo-5,5-dimethylhydantoin (61 mg, 213 mmol) was subsequently added portion-wise and the mixture was stirred at −78° C. for 20 min. Then the cooling bath was replaced by ice-NaCl bath and reaction was stirred for 1 h. The cooled mixture was quenched carefully by the addition of 50% aq. NaOH solution until neutral. Then Na₂S₂O₃ (10% aq. solution, 40 mL) was added. The mixture was filtered to remove the white solid, which was washed with DCM. The filtrate was extracted with DCM. The combined organic layers were washed with water, dried (MgSO₄), filtered and concentrated. The residue was co-distilled with toluene (50 mL) to remove pyridine. Then the crude product was purified by silica gel column chromatography (EtOAc in heptane 0/100 to 30/70) to give 10e (180 mg, 778.71 μmol, 58.46% yield). LCMS: MS m/z (ESI): 232.0 [M+H]⁺.

### Step 5

### (2,2-difluoro-[1,3]dioxolo[4,5-c]pyridin-6-yl)methanol 10f

The mixture of 10e (140 mg, 605.66 μmol) and K₂CO₃ (167.16 mg, 1.21 mmol) in MeOH (8 mL) was heated to reflux for 1 h. Then the solvent was removed under reduced pressure. The residue was dissolved in DCM and water. The organic layer was separated and the aq. layer was extracted with DCM again. The combined organic layers were dried over MgSO₄, filtered and concentrated to give crude 10f (80 mg, 423.02 μmol, 69.84% yield). LCMS: MS m/z (ESI): 190.1 [M+H]⁺.

### Step 6

### 2,2-difluoro-[1,3]dioxolo[4,5-c]pyridine-6-carboxylic acid 10g

TEMPO (1.86 mg, 11.90 μmol) was added to a mixture of 10f (15 mg, 79.32 μmol) in phosphate buffer (pH 7, 4 mL) and CH₃CN (105 mL). The reaction was stirred at 35° C. NaCl (4.64 mg, 79.32 μmol) in NaClO (24.26 mg, 317.27 μmol) and water (2 mL) was added simultaneously in three slow additions each 30 min. The resulting reaction mixture was stirred at 35° C. for 16 hours. The pH was then adjusted to 8 by addition of 1 N aq. NaOH solution. Aq. saturated Na₂S₂O₃ solution was added until the mixture turned white, and stirring was continued for 30 min. The pH was adjusted to 4 by addition of a 1 N HCl solution and the solvent was evaporated under reduced pressure, (Note: the compound decomposed at pH=1). The pH was then adjusted to 2 with 1N HCl and the aqueous residue was extracted with EtOAc, dried (MgSO₄) and concentrated under reduced pressure to give crude 10g (9 mg, 44.31 μmol, 55.87% yield) for next step as is. LCMS: MS m/z (ESI): 204.1 [M+H]⁺.

### Step 7

### 2,2-difluoro-N-(4-methyl-3-(2-morpholino-6-(2-((tetrahydro-2H-pyran-2-yl)oxy)ethoxy)pyridin-4-yl)phenyl)-[1,3]dioxolo[4,5-c]pyridine-6-carboxamide 10 h

To a solution of 10g (9 mg, 44.31 μmol), Int-2 (19 mg, 44.6 μmol) and DIEA (16 mg, 124 μmol) in DMF (2 mL) was added HATU (17 mg, 44.7 μmol), then the reaction was stirred at RT for 1 h. The mixture was diluted with EtOAc and washed with brine. The organic solution was dried over Na₂SO₄ and concentrated under reduced pressure to give crude 10 h for next step. LCMS: MS m/z (ESI): 599.1 [M+H]⁺.

### Step 8

### 2,2-difluoro-N-(3-(2-(2-hydroxyethoxy)-6-morpholinopyridin-4-yl)-4-methylphenyl)-[1,3]dioxolo[4,5-c]pyridine-6-carboxamide 10

Crude 10 h from step 7 was dissolved into MeOH (0.4 mL) and 1.25 mol/L aq. HCl (0.18 mL, 0.225 mmol) was added. The reaction was stirred at RT for 1 h. The mixture was concentrated, and pH was adjusted to 8 with the addition of aq. NaHCO₃, the mixture was extracted with EtOAc, the organic solution was dried and concentrated. The residue was purified by prep-HPLC to give target target 10 (17 mg, 33.07 μmol, 74.6% yield over 2 steps). ¹H NMR (400 MHz, DMSO-d₆): δ 10.65 (s, 1H), 8.80 (s, 1H), 8.21 (s, 1H), 7.82 (d, 1H), 7.79 (d, 1H), 7.27 (d, 1H), 6.26 (s, 1H), 6.04 (s, 1H), 4.25 (t, 1H), 3.71-3.69 (m, 6H), 3.48-3.45 (m, 4H), 2.23 (s, 3H). ¹⁹F NMR (376.5 MHz, DMSO-d₆): δ −48.10. LCMS: MS m/z (ESI): 515.1 [M+H]⁺.

### Example 11

### 2,2-difluoro-N-(2′-(2-hydroxyethoxy)-2-methyl-6′-morpholino-[3,4′-bipyridin]-5-yl)benzo[d][1,3]dioxole-5-carboxamide 11

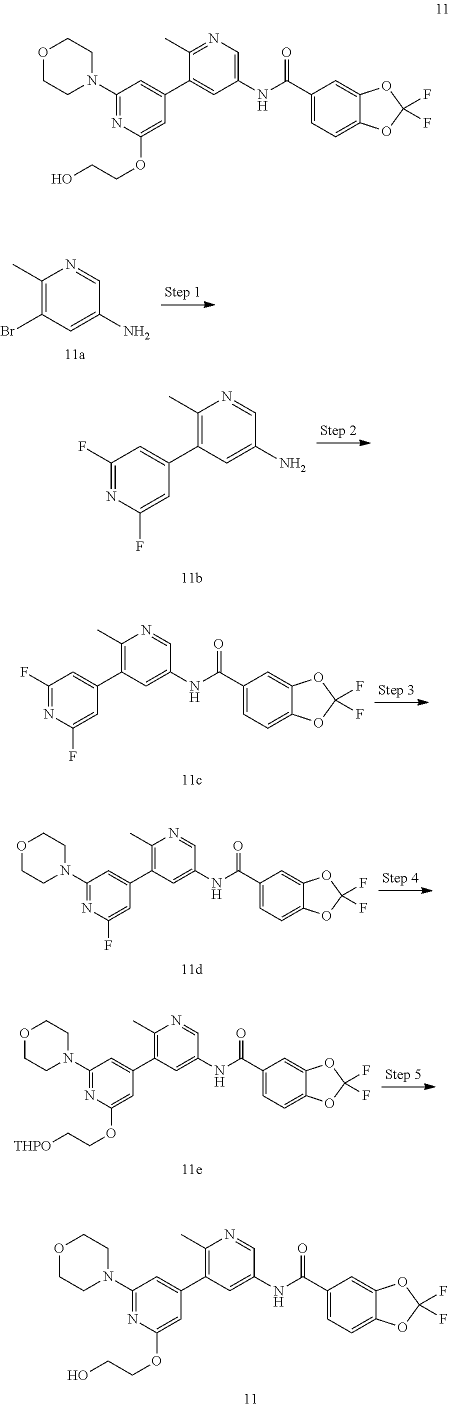

### Step 1

### 2′,6′-difluoro-2-methyl[3,4′-bipyridin]-5-amine 11b

To a solution of 2,6-Difluoropyridine-4-boronic acid, pinacol ester (250 mg, 1.03 mmol) in H₂O (1 mL) and 1,4-dioxane (5 mL) was added 5-bromo-6-methylpyridin-3-amine 11a (194 mg, 1.03 mmol), K₂CO₃ (430 mg, 3.11 mmol) and Pd(dppf)Cl₂ (85 mg, 103.7 μmol), then the reaction was stirred at 90° C. under Ar for 1.5 hrs. The reaction solution was cooled down to RT. The mixture was diluted with water and the resulting solution was extracted with EtOAc (20 mL×3). The organic phase was dried over anhydrous Na₂SO₄, filtered and evaporated. The residue was purified by silica gel column chromatography (MeOH:DCM=100:1 to 20:1) to give the title compound 11b (191 mg, 0.864 mmol, 83.9% yield). LCMS: MS m/z (ESI): 222.0 [M+H]⁺.

### Step 2

### N-(2′,6′-difluoro-2-methyl-[3,4′-bipyridin]-5-yl)-2,2-difluorobenzo[d][1,3]dioxole-5-carboxamide 11c

The solution of 11b (137 mg, 619.33 μmol) in DMF (4 mL) was cooled to 0° C. before 2,2-difluoro-1,3-benzodioxole-5-carboxylic acid (125.17 mg, 619.33 μmol), HATU (353.23 mg, 929.00 μmol) and TEA (188.01 mg, 1.86 mmol) were added. The mixture was stirred at RT for 4 h. The mixture was diluted with water and extracted with EtOAc (50 mL×2). The combined organic layer was washed with brine, dried over anhydrous Na₂SO₄ and concentrated. The residue was purified by silica gel chromatography (hexane/EtOAc=5/1) to afford 11c (200 mg, 493.46 μmol, 79.68% yield). LCMS: MS m/z (ESI): 406.0 [M+H]⁺.

### Step 3

### 2,2-difluoro-N-(2′-fluoro-2-methyl-6′-morpholino-[3,4′-bipyridin]-5-yl)benzo[d][1,3]dioxole-5-carboxamide 11d

To a solution of 11c (200 mg, 493.46 μmol) and morpholine (128.97 mg, 1.48 mmol) in DMSO (4 mL) was added K₂CO₃ (136.19 mg, 986.92 μmol), the mixture was stirred at 80° C. for 16 h. The reaction mixture was cooled to rt, diluted with water and extracted with EtOAc (50 mL×2), the combined organic layer was washed with brine, dried over anhydrous Na₂SO₄ and concentrated. The residue was purified by silica gel chromatography (hexane/EtOAc=4/1) to afford 11d (150 mg, 316.84 μmol, 64.21% yield). LCMS: MS m/z (ESI): 473.1 [M+H]⁺.

### Step 4

### 2,2-difluoro-N-(2-methyl-2′-morpholino-6′-(2-((tetrahydro-2H-pyran-2-yl)oxy)ethoxy)-[3,4′-bipyridin]-5-yl)benzo[d][1,3]dioxole-5-carboxamide 11e

To a solution of 2-((tetrahydro-2H-pyran-2-yl)oxy)ethan-1-ol (92.63 mg, 633.68 μmol) in DMF (4 mL) was added NaH (47.53 mg, 792.10 μmol, 60% wt) at 0° C. The mixture was stirred at 0° C. for 30 min, then the solution of 11d (150 mg, 316.84 μmop in DMF (1 mL) was added dropwise. The mixture was stirred at 80° C. for 16 h, then the reaction mixture was cooled to RT, diluted with water and extracted with EtOAc (50 mL×2). The combined organic layer was washed with brine, dried over anhydrous Na₂SO₄ and concentrated. The residue was purified by silica gel chromatography (hexane/EtOAc=2/1) to afford 11e (110 mg, 183.46 μmol, 57.90% yield). LCMS: MS m/z (ESI): 597.3 [M−H]⁻.

### Step 5

### 2,2-difluoro-N-(2′-(2-hydroxyethoxy)-2-methyl-6′-morpholino-[3,4′-bipyridin]-5-yl)benzo[d][1,3]dioxole-5-carboxamide 11

To a solution of 11e (110 mg, 183.76 μmol, 8126232) in DCM (2 mL, richjoint) was added TFA (1 mL, Accela) at 0° C., the mixture was stirred at rt for 1 h. TLC analysis indicated that SM was consumed. The mixture was diluted with H₂O (5 mL), adjusted pH=7-8 with saturated NaHCO₃ and extracted with EtOAc (50 mL×2), the combined organic layer was washed with brine, dried over anhydrous Na₂SO₄ and concentrated. The residue was purified by SGC (DCM/MeOH=50/1) to afford 11 (65 mg, 126.34 μmol, 68.75% yield). ¹H NMR (400 MHz, DMSO-d₆): δ 10.51 (s, 1H), 8.84 (d, 2.4 Hz, 1H), 8.03-7.99 (m, 2H), 7.92-7.89 (m, 1H), 7.61 (d, 8.4 Hz, 1H), 6.32 (s, 1H), 6.10 (s, 1H), 4.48 (br, 1H), 4.26 (t, 5.2 Hz, 1H), 3.72-3.69 (m, 6H), 3.47 (br, 4H), 2.42 (s, 3H). LCMS: MS m/z (ESI): 515.5 [M+H]⁺.

### Example 12

### 2,2-difluoro-N-(2′-(2-hydroxyethoxy)-5-methyl-6′-morpholino-[4,4′-bipyridin]-2-yl)benzo[d][1,3]dioxole-5-carboxamide 12

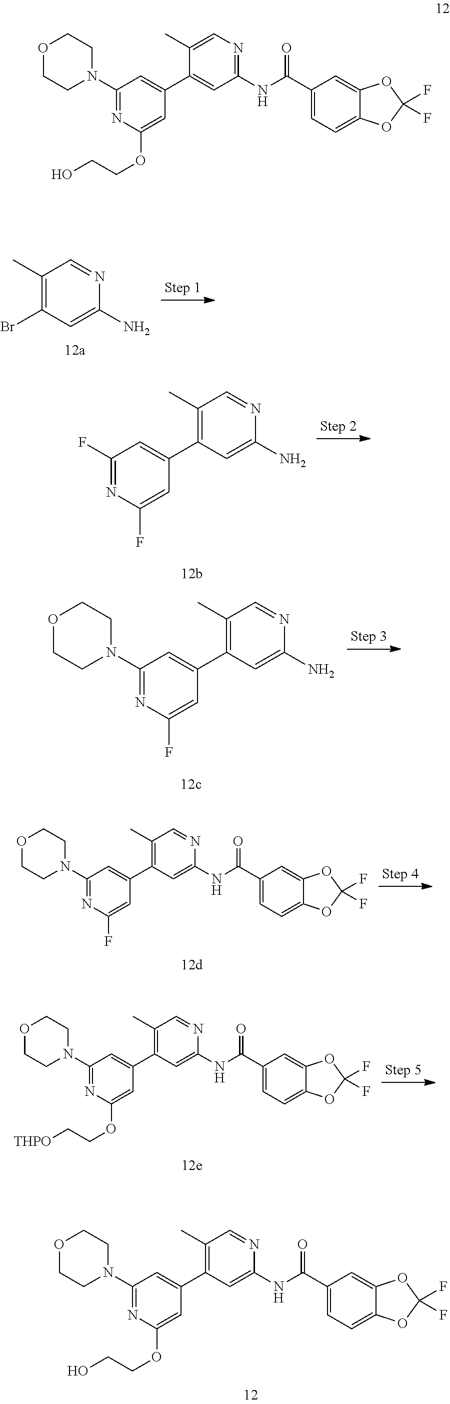

### Step 1

### 2′,6′-difluoro-5-methyl-[4,4′-bipyridin]-2-amine 12b

To a solution of 2,6-difluoropyridine-4-boronic acid, pinacol ester (322 mg, 1.34 mmol) in DMSO (3.0 mL) was added 4-bromo-5-methylpyridin-2-amine 12a (250 mg, 1.34 mmol, AstaTech), K₃PO₄ (860 mg, 4.06 mmol) and Pd(dppf)Cl₂.CH₂Cl₂ (55 mg, 67 μmol), then the reaction was stirred at 100° C. under Ar for 1.5 hrs. The reaction solution was cooled down to RT. The mixture was diluted with water and the resulting solution was extracted with EtOAc (20 mL×3). The organic phase was dried over anhydrous Na₂SO₄, filtered and evaporated. The residue was purified by silica gel column chromatography (MeOH:DCM=100:1 to 20:1) to give the title compound 12b (272 mg, 91.8% yield). LCMS: MS m/z (ESI): 222.0 [M+H]⁺.

### Step 2

### 2′-fluoro-5-methyl-6′-morpholino-[4,4′-bipyridin]-2-amine 12c

To a solution of 12b (272 mg, 1.23 mmol) in DMSO (10 mL) was added K₂CO₃ (370 mg, 2.68 mmol) and morpholine (0.47 mL, 5.35 mmol), then the mixture was stirred at 40° C. for 40 min. After the reaction complete, the reaction solution was cooled down to RT. The solution was diluted with water (10 mL) and the resulting solution was extracted with EtOAc (30 mL×3). The organic phase was dried over anhydrous Na₂SO₄, filtered and evaporated. The residue was purified by silica gel column chromatography (MeOH:DCM=100:1 to 10:1) to give the title compound 12c (309 mg, 86.9% yield). LCMS: MS m/z (ESI): 289.2 [M+H]⁺.

### Step 3

### 2,2-difluoro-N-(2′-fluoro-5-methyl-6′-morpholino-[4,4′-bipyridin]-2-yl)benzo[d][1,3]dioxole-5-carboxamide 12d

To a solution of 12c (309 mg, 1.07 mmol), 2,2-difluoro-1,3-benzodioxole-5-carboxylic acid (217 mg, 1.07 mmol), DIEA (0.4 mL, 4.52 mmol) in DMF (4 mL) was added HATU (407 mg, 1.07 mmol). The mixture was stirred at RT for 5 h, LCMS showed ˜30% of starting material was consumed. The mixture was diluted with EtOAc and the mixture was washed with brine. The organic solution was dried and concentrated. The residue was purified by silica gel column chromatography (hexane/EtOAc) to give the title compound 12d (141 mg, 27.9% yield). LCMS: MS m/z (ESI): 473.0 [M+H]⁺.

### Step 4

### 2,2-difluoro-N-(5-methyl-2′-morpholino-6′-(2-((tetrahydro-2H-pyran-2-yl)oxy)ethoxy)-[4,4′-bipyridin]-2-yl)benzo[d][1,3]dioxole-5-carboxamide 12e

To a solution of 2-((tetrahydro-2H-pyran-2-yl)oxy)ethan-1-ol (88 mg, 0.6 mmol) in 1,4-dioxane (5 mL) was added NaH (48 mg, 60% wt, 1.2 mmol) portion wisely at 0° C. After stirring at this temperature for 10 min, a solution of 12d (141 mg, 0.299 mmol) in 1,4-dioxane (2 mL) was added and the reaction was stirred at 70° C. for 16 hrs. The reaction solution was diluted with brine (15 mL). The resulting solution was extracted with EtOAc (25 mL×3). The organic phase was dried over anhydrous Na₂SO₄, filtered and evaporated to give a crude product which was purified by silica gel column chromatography (hexane/EtOAc) to give the title compound 12e (80 mg, 44.5% yield). LCMS: MS m/z (ESI): 599.2 [M+H]⁺.

### Step 5

### 2,2-difluoro-N-(2′-(2-hydroxyethoxy)-5-methyl-6′-morpholino-[4,4′-bipyridin]-2-yl)benzo[d][1,3]dioxole-5-carboxamide 12

To a solution of 12e (80 mg, 0.134 mmol) in THF (2 mL) was added 1.25N HCl (0.70 ml, 0.875 mmol). The mixture reaction was stirred at RT for 1.5 hr. The reaction solution was diluted with saturated NaHCO₃ (25 mL) and extracted with EtOAc (25 mL×3). The organic phase was dried over anhydrous Na₂SO₄, filtered and purified by prep-HPLC to give the title compound 12 (11.4 mg, 39.7% yield). ¹H NMR (400 MHz, Methanol-d4): δ 8.29 (s, 1H), 8.05 (s, 1H), 7.90-7.85 (m, 2H), 7.38 (d, 1H), 6.27 (s, 1H), 6.15 (s, 1H), 4.40 (t, 2H), 3.89 (t, 2H), 3.85-3.80 (m, 4H), 3.58-3.54 (m, 4H), 2.28 (s, 3H). ¹⁹F NMR (376.5 MHz, Methanol-d4): δ −52.05. LCMS: MS m/z (ESI): 515.1 [M+H]⁺.

### Example 13

### N-(3-(2-(2-hydroxyethoxy)-6-morpholinopyridin-4-yl)-4-methylphenyl)-2,3-dihydrobenzo[b][1,4]dioxine-6-carboxamide 13

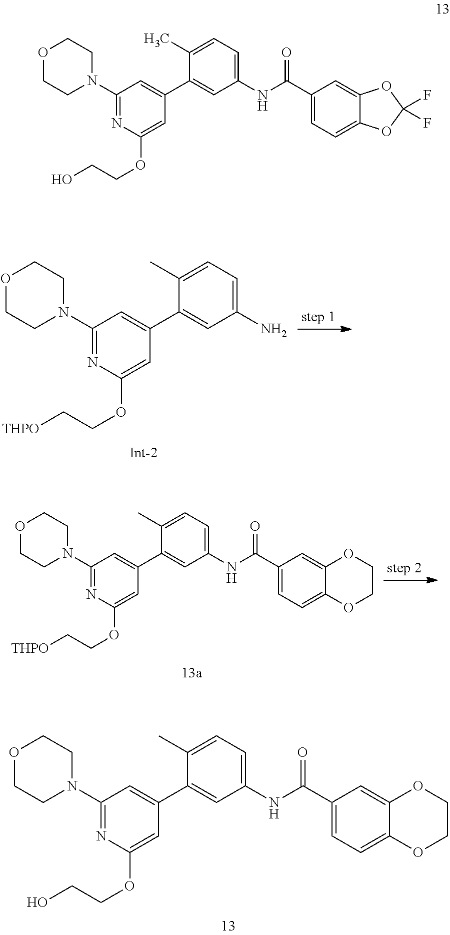

### Step 1

### N-(4-methyl-3-(2-morpholino-6-(2-((tetrahydro-2H-pyran-2-yl)oxy)ethoxy)pyridin-4-yl)phenyl)-2,3-dihydrobenzo[b][1,4]dioxine-6-carboxamide 13a

To a solution of Int-2 (86 mg, 0.208 mmol), 2,3-dihydro-1,4-benzodioxine-6-carboxylic acid (38 mg, 0.211 mmol, Combi-Blocks) and DIPEA (0.10 mL, 0.565 mmol) in DMF (2 mL) was added HATU (79 mg, 0.208 mmol), then the mixture was stirred at RT for 2 hr. The mixture was diluted with water and extracted with EtOAc. The organic solution was concentrated, and the residue was purified by silica gel chromatagraphy (EtOAc/hexane) to give target 13a (46 mg, 38.4% yield). MS m/z (ESI): 576.2 [M+H]⁺.

### Step 2

### N-(3-(2-(2-hydroxyethoxy)-6-morpholinopyridin-4-yl)-4-methylphenyl)-2,3-dihydrobenzo[b][1,4]dioxine-6-carboxamide 13

To a solution of 13a (46 mg, 0.080 mmol) in THF (2 mL) was added 6N aq. HCl (0.5 mL, 3.0 mmol) slowly at 0° C. Then the mixture was stirred at RT for 30 min. The mixture was cooled at 0° C. again and carefully neutralized by saturated aq. NaHCO₃, then extracted with EtOAc. The organic solution was dried and concentrated. The resulted residue was purified by silica gel chromatagraphy (0-100% EtOAc/hexane) to give target 13 (23.2 mg, 59.0% yield). ¹H NMR (400 MHz, Methanol-d4): δ 7.51-7.41 (m, 2H), 7.41-7.27 (m, 2H), 7.15 (d, 1H), 6.84 (d, 1H), 6.13 (d, 1H), 6.02 (d, 1H), 4.32-4.24 (m, 2H), 4.24-4.10 (m, 4H), 3.85-3.73 (m, 2H), 3.74-3.59 (m, 4H), 3.48-3.30 (m, 4H), 2.15 (s, 3H). MS m/z (ESI): 492.2 [M+H]⁺.

### Biological Assays

The present disclosure will be further described with reference to the following test examples, but the examples should not be considered as limiting the scope of the disclosure.

cRAF Inhibition Assay

Activity of human recombinant cRAF were assessed in vitro using time-resolved fluorescence resonance energy transfer (TR-FRET) assay. The assay was performed according to the kit instructions. Briefly, the cRAF enzyme (BPS Bioscience, cat #40008) was diluted to 0.027 ng/μL using the 1× kinase buffer A (Life Tech, cat #PV6135, 5× dilution with H₂O); the Fluorescein labeled-MAP2K1 (Life Tech, Cat #PV4812) was diluted to 0.5 μM; ATP (Life Tech, cat #PV3227) was diluted to 20 μM; 2.54, of each diluted reagent was added to a 384-well plate (PerkinElmer, ProxiPlate-384 Plus, 6008280) and mixed together; 2.54, serial diluted compound were added into the mixture. 104, 400-fold diluted LanthaScreen Tb-pMAP2K1 (pSer217/221) antibody (Life Tech, cat #PV4817) was then added to each kinase reaction. The reactions were incubated at Room Temperature for 1 hour. The plate was then read on PHERAstar FSX using 340/490/520 module. Inhibition data were calculated by comparison to vehicle control wells for 0% inhibition and non-stimulated control wells for 100% inhibition. Dose response curves were then generated to determine the concentration required to suppress 50% of enzymatic response (IC₅₀) as derived by non-linear regression analysis using GraphPad Prism.

BRAF^{WT} Inhibition Assay

Activity of human recombinant BRAF were assessed in vitro using time-resolved fluorescence resonance energy transfer (TR-FRET) assay. The assay was performed according to the kit instructions. Briefly, the BRAF enzyme (BPS Bioscience, cat #40065) was diluted to 0.027 ng/μL using the 1× kinase buffer A (Life Tech, cat #PV6135, 5× dilution with H₂O); the Fluorescein labeled-MAP2K1 (Life Tech, Cat #PV4812) was diluted to 0.5 μM; ATP (Life Tech, cat #PV3227) was diluted to 20 μM; 2.54, of each diluted reagent was added to a 384-well plate (PerkinElmer, ProxiPlate-384 Plus, 6008280) and mixed together; 2.54, serial diluted compound were added into the mixture. 104, 400-fold diluted LanthaScreen Tb-pMAP2K1 (pSer217/221) antibody (Life Tech, cat #PV4817) was then added to each kinase reaction. The reactions were incubated at Room Temperature for 1 hour. The plate was then read on PHERAstar FSX using 340/490/520 module. Inhibition data were calculated by comparison to vehicle control wells for 0% inhibition and non-stimulated control wells for 100% inhibition. Dose response curves were then generated to determine the concentration required to suppress 50% of enzymatic response (IC₅₀) as derived by non-linear regression analysis using GraphPad Prism.

BRAF^{V600E} Inhibition Assay

Activity of human recombinant V600E mutant BRAF were assessed in vitro using time-resolved fluorescence resonance energy transfer (TR-FRET) assay. The assay was performed according to the kit instructions. Briefly, the V600E mutant BRAF enzyme (BPS Bioscience, cat #40533) was diluted to 0.027 ng/μL using the 1× kinase buffer A (Life Tech, cat #PV6135, 5× dilution with H₂O); the Fluorescein labeled-MAP2K1 (Life Tech, Cat #PV4812) was diluted to 0.5 μM; ATP (Life Tech, cat #PV3227) was diluted to 20 μM; 2.5 μl of each diluted reagent was added to a 384-well (PerkinElmer, ProxiPlate-384 Plus, 6008280) plate and mixed together; 2.54, serial diluted compounds were added into the mixture. 104, 400-fold diluted LanthaScreen Tb-pMAP2K1 (pSer217/221) antibody (Life Tech, cat #PV4817) was then added to each kinase reaction. The reactions were incubated at Room Temperature for 1 hour. The plate was then read on PHERAstar FSX using 340/490/520 module. Inhibition data were calculated by comparison to vehicle control wells for 0% inhibition and non-stimulated control wells for 100% inhibition. Dose response curves were then generated to determine the concentration required to suppress 50% of enzymatic response (IC₅₀) as derived by non-linear regression analysis using GraphPad Prism.

NCI-H358 Cancer Cell Growth Inhibition Assay

Activity of human NCI-H358 (KRAS^{G12C}) non-small cell lung cancer cell growth inhibition was assessed in vitro using CellTiter-Glo® Luminescent Cell Viability Assay. The assay was performed according to the kit instructions (Promega, cat #G7570). Briefly, NCI-H358 cells (ATCC, #CRL-5807) were seeded in 96-well plates at a density of 2,500 cells/well in and cells were cultured overnight in a humidified, 5% CO₂ cell culture incubator at 37° C. 10 μL serial diluted compounds were added into each cell-culture well and incubated for 3 days. 100 μL CellTiter-Glo® Reagent was added into each well. The reactions were incubated at Room Temperature for 10 minutes, and luminescence signals were recorded on a Tecan Luminescence Plate Reader. Inhibition data were calculated by comparison to vehicle control wells for 0% inhibition and non-stimulated control wells for 100% inhibition. Dose response curves were then generated to determine the concentration required to suppress 50% of cellular growth response (GI₅₀) as derived by non-linear regression analysis using GraphPad Prism.

Calu6 Cancer Cell Growth Inhibition Assay

Activity of human Calu6 (KRAS^{Q16K}) cancer cell growth inhibition was assessed in vitro using CellTiter-Glo® Luminescent Cell Viability Assay. The assay was performed according to the kit instructions (Promega, cat #G7570). Briefly, Calu6 cells (ATCC, #HTB-56™) were seeded in 96-well plates at a density of 2,500 cells/well in 90 and cells were cultured overnight in a humidified, 5% CO₂ cell culture incubator at 37° C. 10 μL serial diluted compounds were added into each cell-culture well and incubated for 3 days. 100 μL CellTiter-Glo® Reagent was added into each well. The reactions were incubated at Room Temperature for 10 minutes, and luminescence signals were recorded on a Tecan Luminescence Plate Reader. Inhibition data were calculated by comparison to vehicle control wells for 0% inhibition and non-stimulated control wells for 100% inhibition. Dose response curves were then generated to determine the concentration required to suppress 50% of cellular growth response (GI₅₀) as derived by non-linear regression analysis using GraphPad Prism.

A375 Cell Growth Inhibition Assay

Activity of human A375 (BRAF^{V600E}) malignant melanoma cell growth inhibition was assessed in vitro using CellTiter-Glo® Luminescent Cell Viability Assay. The assay was performed according to the kit instructions (Promega, cat #G7570). Briefly, A375 cells (ATCC, #CRL-1619™) were seeded in 96-well plates at a density of 2,500 cells/well in 90 and cells were cultured overnight in a humidified, 5% CO₂ cell culture incubator at 37° C. 10 μL serial diluted compounds were added into each cell-culture well and incubated for 3 days. 100 CellTiter-Glo® Reagent was added into each well. The reactions were incubated at Room Temperature for 10 minutes, and luminescence signals were recorded on a Tecan Luminescence Plate Reader. Inhibition data were calculated by comparison to vehicle control wells for 0% inhibition and non-stimulated control wells for 100% inhibition. Dose response curves were then generated to determine the concentration required to suppress 50% of cellular growth response (GI₅₀) as derived by non-linear regression analysis using GraphPad Prism.

LXH254 is Novartis clinical stage highly selective RAF inhibitor with nanomolar potency for bRAF and cRAF. When compared to LXH254, Examples 1, 2, 3, 6, 7, 8, 9, 10, 11 and 13 all showed similar enzymatic potency. Furthermore, in H358 cancer line, Examples 1, 7, 8, 9, 10 and 13 demonstrated better or similar cell growth inhibition. Example 1 also exhibits excellent cellular potency among different cancer cell lines, including H358, A375 and Calu6.

TABLE 1
RAF Kinase Inhibition

| Example | cRAF | BRAF^{WT} | BRAF^{V600E} |
|---|---|---|---|
| No | IC₅₀ (nM) | IC₅₀ (nM) | IC₅₀ (nM) |
| LXH254 | 13 | 38 | 43 |
| 1 | 15 | 87 | 32.5 |
| 2 | 7.9 | 38.5 | 18 |
| 3 | 27 | 170 | 88 |
| 4 | 51 | 260 | 96 |
| 5 | 39 | 92 | 65 |
| 6 | 3.9 | 26 | 8.4 |
| 7 | 10 | 46 | 18 |
| 8 | 25 | 140 | 62 |
| 9 | 6.7 | 42 | 18 |
| 10 | 25 | 95 | 55 |
| 11 | 10 | 70 | 39 |
| 13 | 4.7 | 31 | 13 |

TABLE 2
Cancer Cell Growth Inhibition

| Example No | H358 GI₅₀ (nM) | A375 GI₅₀ (nM) | Calu6 GI₅₀ (nM) |
|---|---|---|---|
| LXH254 | 3000 | 890 | 920 |
| 1 | 480 | 150 | 64 |
| 7 | 2800 | — | — |
| 8 | 220 | — | — |
| 9 | 1600 | — | — |
| 10 | 85 | — | — |
| 13 | 1200 | — | — |

The foregoing embodiments and examples are provided for illustration only and are not intended to limit the scope of the invention. Various changes and modifications to the disclosed embodiments will be apparent to those skilled in the art based on the present disclosure, and such changes and modifications may be made without departure from the spirit and scope of the present invention. All patent or non-patent references cited are incorporated herein by reference in their entireties without admission of them as prior art.

What is claimed is:

1. A compound of formula (I): 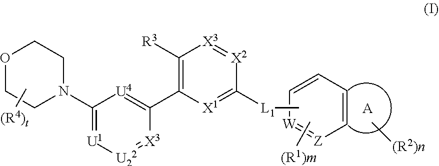
or a tautomer, racemate, enantiomer, or diastereomer thereof, or a pharmaceutically acceptable salt, solvate, or prodrug thereof, wherein:
ring A is an optionally substituted cycloalkyl or heterocyclyl fused to the adjacent aromatic ring;
W and Z are identical or different, and each is independently C or N, provided that W and Z are not both N at the same time;
R¹ at each occurrence is identical or different, and each is independently selected from hydrogen, hydroxyl, halogen, alkyl, alkenyl, alkynyl, haloalkyl, hydroxyalkyl, cyano, amino, —NHR⁶, —N(R⁶)₂, —OR⁷, —SO₂R⁸, —S(═NH)(═O)R⁸, cycloalkyl, heterocyclyl, aryl, and heteroaryl;
R² at each occurrence is identical or different, and each is independently selected from hydrogen, hydroxyl, halogen, alkyl, alkenyl, alkynyl, haloalkyl, hydroxyalkyl, cyano, amino, —NHR⁶, —N(R⁶)₂, —OR⁷, —SO₂R⁸, —S(═NH)(═O)R⁸, cycloalkyl, heterocyclyl, aryl, and heteroaryl;
L₁ is —C(═O)—NR⁰— or —NR⁰—C(═O)—, wherein R⁰ is selected from hydrogen, alkyl, haloalkyl, and hydroxyalkyl;
X¹, X² and X³ are identical or different, and each is independently selected from CH, N, and NO;
R³ is selected from hydrogen, hydroxyl, halogen, alkyl, alkoxy, haloalkyl, hydroxyalkyl, cyano, and amino;
U¹, U², U³, and U⁴ are identical or different, and each is independently CR⁵ or N, wherein R⁵ at each occurrence is independently selected from hydrogen, halogen, alkyl, alkenyl, alkynyl, alkoxy, hydroxyl, cyano, amino, —O-L₂-OH, —NHR⁶, —N(R⁶)₂, —SO₂R⁸, —NHSO₂R⁸, —NHC(O)R⁸, —NR⁶CO₂R⁸, cycloalkyl, heterocyclyl, aryl, heteroaryl, —O-cycloalkyl, —O-heterocyclyl, —O-aryl, and —O-heteroaryl; wherein the alkyl, alkoxy, cycloalkyl, heterocyclyl, aryl and heteroaryl are each optionally substituted with one or more groups selected from oxo, hydroxyl, halogen, alkyl, haloalkyl, hydroxyalkyl, cyano, amino, and alkoxy; wherein L₂ is alkylene;
Y is selected from O, S, S(═O), and SO₂;
R⁴ at each occurrence is identical or different, and each is selected from hydrogen, hydroxyl, halogen, alkyl, alkoxy, oxo, haloalkyl, hydroxyalkyl, cyano, and amino;
R⁶, R⁷ and R⁸ at each occurrence are identical or different, and each is selected from alkyl, alkenyl, alkynyl, haloalkyl, hydroxyalkyl, cycloalkyl, heterocyclyl, aryl, and heteroaryl;
m is 0, 1 or 2;
n is 0, 1, 2, 3 or 4; and
t is 0, 1, 2, 3 or 4.

2. The compound of claim 1, wherein Y is O; and ring A is a C₄-C₆ cycloalkyl or 4- to 6-membered heterocyclyl.

3. The compound of claim 1, being a compound of formula (II): 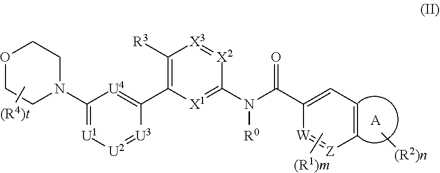
or a tautomer, racemate, enantiomer, or diastereomer thereof, or a pharmaceutically acceptable salt, solvate or prodrug thereof, wherein:
W, Z, R⁰, X¹, X², X³, U¹, U², U³, U⁴, ring A, R¹ to R⁴, m, n and t are as defined in claim 1.

4. The compound of claim 1, being a compound of formula (III): 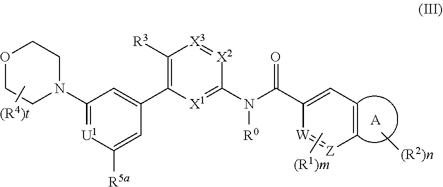
or a tautomer, racemate, enantiomer, or diastereomer thereof, or a pharmaceutically acceptable salt, solvate or prodrug thereof, wherein:
R^{5a} is —O-L₂-OH;
L₂ is alkylene; and
W, Z, R⁰, X¹, X², X³, U¹, ring A, R¹ to R⁴, m, n and t are as defined in claim 1.

5. The compound of claim 1, wherein 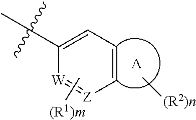
is selected from 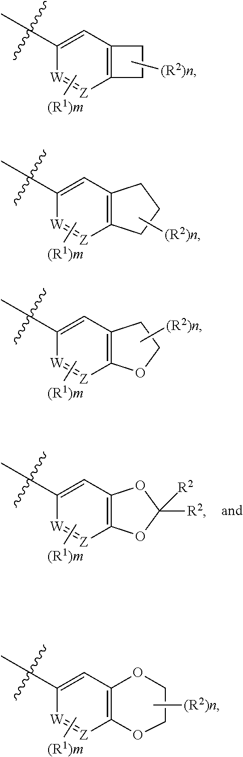
wherein W, Z, R², m and n are as defined in claim 1.

6. The compound of claim 1, being a compound of formula (IV-1): 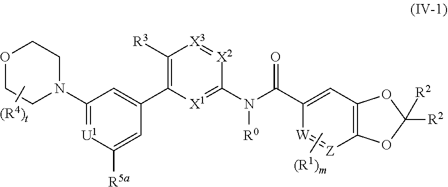
or a tautomer, racemate, enantiomer, or diastereomer thereof, or a pharmaceutically acceptable salt, solvate or prodrug thereof, wherein:
R^{5a} is —O-L₂-OH;
L₂ is alkylene; and
W, Z, R⁰, X¹, X², X³, U¹, R¹ to R⁴, m and t are as defined in claim 1.

7. The compound of claim 1, being a compound of formula (IV-2): 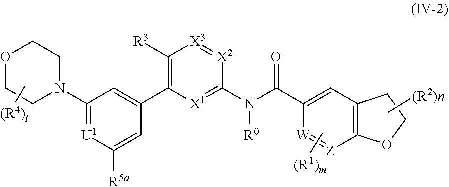
or a tautomer, racemate, enantiomer, or diastereomer thereof, or a pharmaceutically acceptable salt, solvate or prodrug thereof, wherein:
R^{5a} is —O-L₂-OH;
L₂ is alkylene; and
W, Z, R⁰, X¹, X², X³, U¹, R¹ to R⁴, m, n and t are as defined in claim 1.

8. The compound of claim 1, being a compound of formula (IV-3): 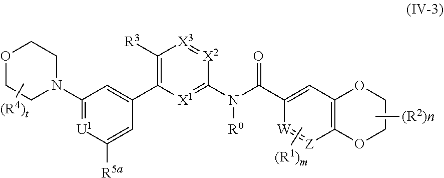
or a tautomer, racemate, enantiomer, or diastereomer thereof, or a pharmaceutically acceptable salt, solvate or prodrug thereof, wherein:
R^{5a} is —O-L₂-OH;
L₂ is alkylene; and
W, Z, R⁰, X¹, X², X³, U¹, R¹ to R⁴, m, n and t are as defined in claim 1.

9. The compound of claim 1, being a compound of formula (IV-4): 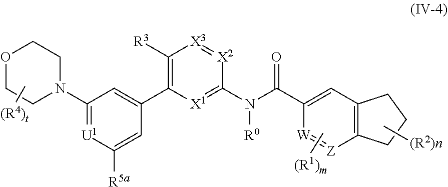
or a tautomer, racemate, enantiomer, or diastereomer thereof, or a pharmaceutically acceptable salt, solvate or prodrug thereof, wherein:
R^{5a} is —O-L₂-OH;
L₂ is alkylene; and
W, Z, R⁰, X¹, X², X³, U¹, R¹ to R⁴, m, n and t are as defined in claim 1.

10. The compound of claim 1, or a tautomer, racemate, enantiomer, or diastereomer thereof, or a pharmaceutically acceptable salt, solvate or prodrug thereof, wherein X¹ is CH, X² and X³ are identical or different, and each is independently selected from CH and N, provided that X² and X³ are not N at the same time.

11. The compound of claim 10, or a tautomer, racemate, enantiomer, or diastereomer thereof, or a pharmaceutically acceptable salt, solvate or prodrug thereof, wherein U¹ is CR⁵ or N; and R⁵ is selected from hydrogen, halogen, and alkyl.

12. The compound of claim 11, or a tautomer, racemate, enantiomer, or diastereomer thereof, or a pharmaceutically acceptable salt, solvate or prodrug thereof, wherein:
Y is O;
R⁰ is hydrogen or alkyl;
R¹ at each occurrence is independently selected from hydrogen, halogen, and alkyl;
R² at each occurrence is independently selected from hydrogen, hydroxyl, halogen, alkyl, alkenyl, alkynyl, haloalkyl, and hydroxyalkyl;
R³ is selected from hydrogen, hydroxyl, halogen, alkyl, alkenyl, alkynyl, haloalkyl and hydroxyalkyl; and
R⁴ is hydrogen.

13. The compound of claim 1, or a pharmaceutically acceptable salt thereof, wherein the compound is selected from:

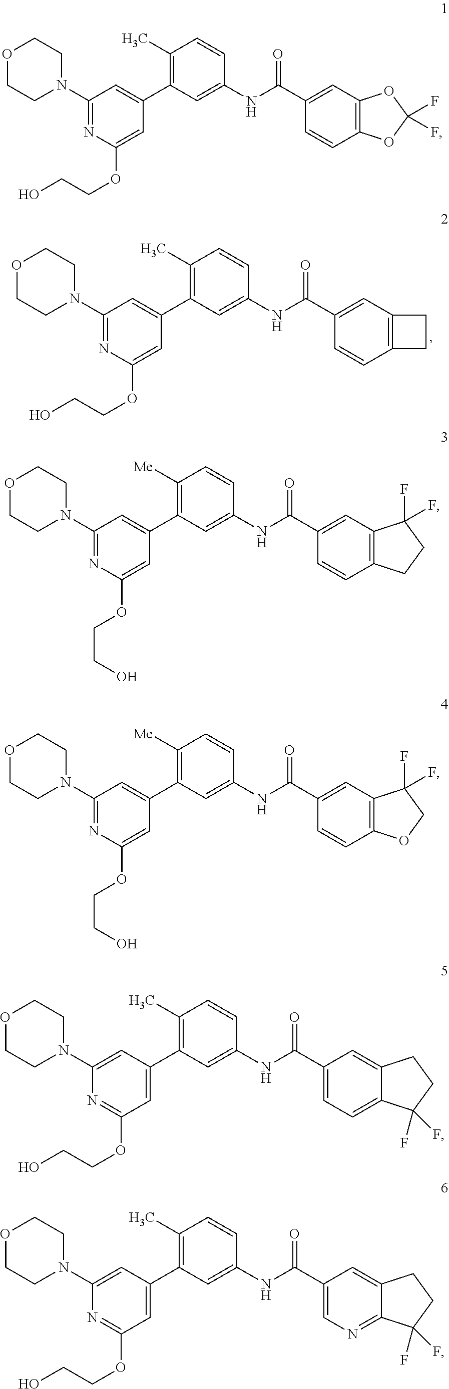 [SMILES: Cc1ccc(NC(=O)c2ccc3c(c2)OC(F)(F)O3)cc1-c1cc(OCCO)nc(N2CCOCC2)c1 ; Cc1ccc(NC(=O)c2ccc3c(c2)CC3)cc1-c1cc(OCCO)nc(N2CCOCC2)c1 ; Cc1ccc(NC(=O)c2ccc3c(c2)C(F)(F)CC3)cc1-c1cc(OCCO)nc(N2CCOCC2)c1 ; Cc1ccc(NC(=O)c2ccc3c(c2)C(F)(F)CO3)cc1-c1cc(OCCO)nc(N2CCOCC2)c1 ; Cc1ccc(NC(=O)c2ccc3c(c2)CCC3(F)F)cc1-c1cc(OCCO)nc(N2CCOCC2)c1 ; Cc1ccc(NC(=O)c2cnc3c(c2)CCC3(F)F)cc1-c1cc(OCCO)nc(N2CCOCC2)c1]

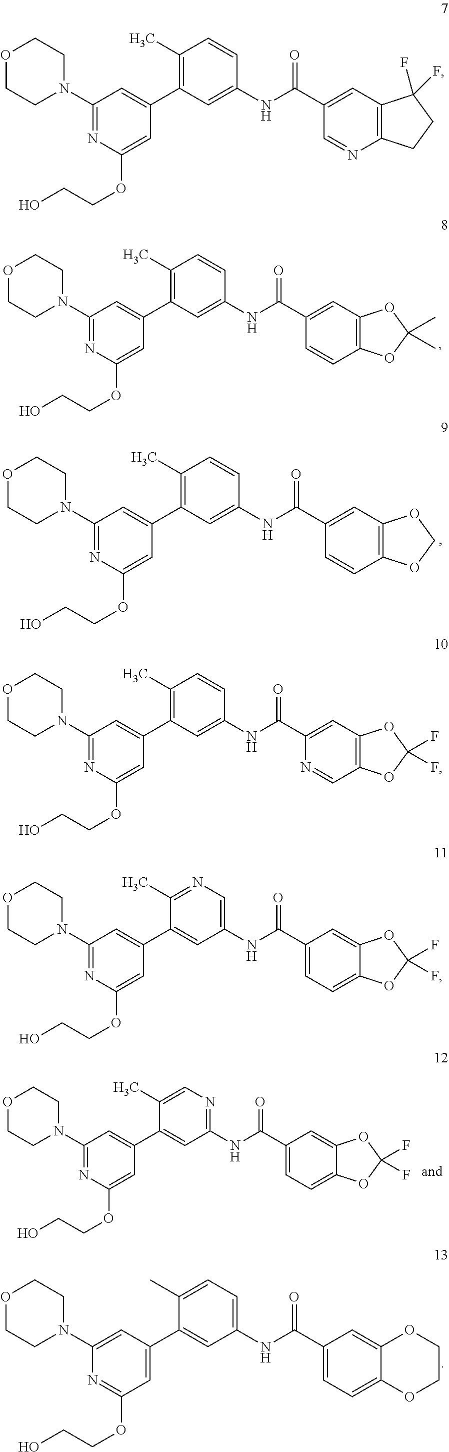

14. A compound of formula (IIIA): 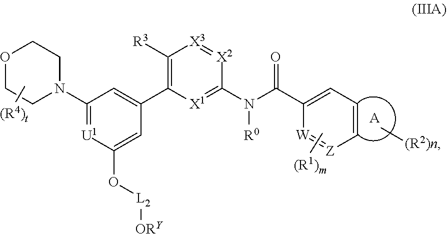
or a tautomer, racemate, enantiomer, or diastereomer thereof, or a pharmaceutically acceptable salt, solvate, or prodrug thereof, wherein:
L₂ is alkylene;
R^{Y} is a hydroxyl protecting group; and
W, Z, R⁰, X¹, X², X³, U¹, ring A, R¹ to R⁴, m, n and t are as defined in claim 4.

15. The compound of claim 14, or a tautomer, racemate, enantiomer, or diastereomer thereof, or a pharmaceutically acceptable salt, solvate or prodrug thereof, wherein the compound is selected from:

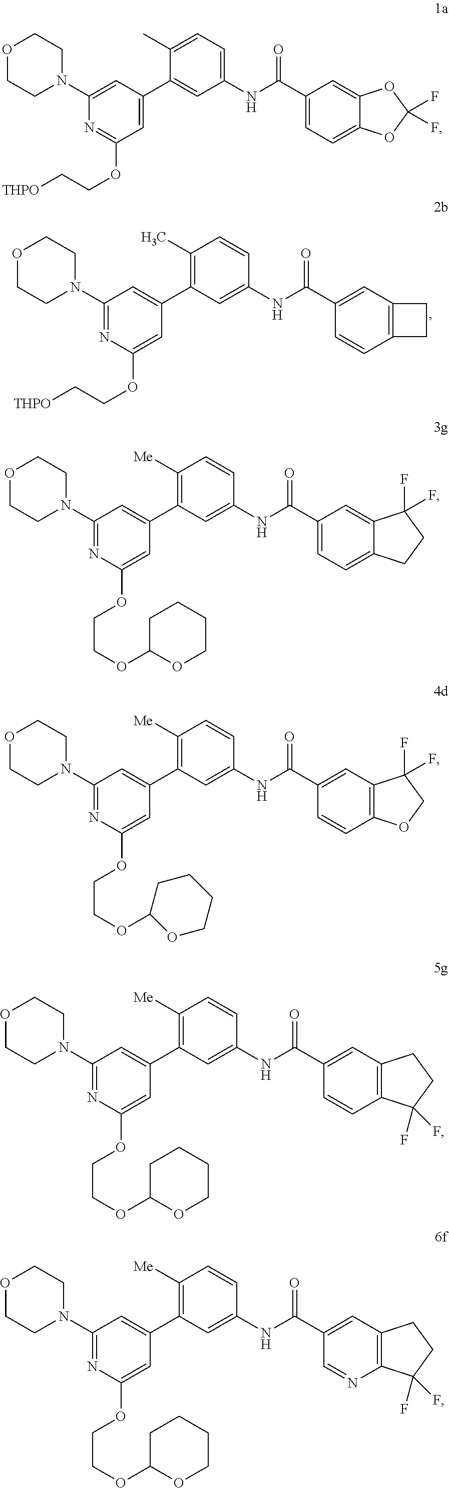

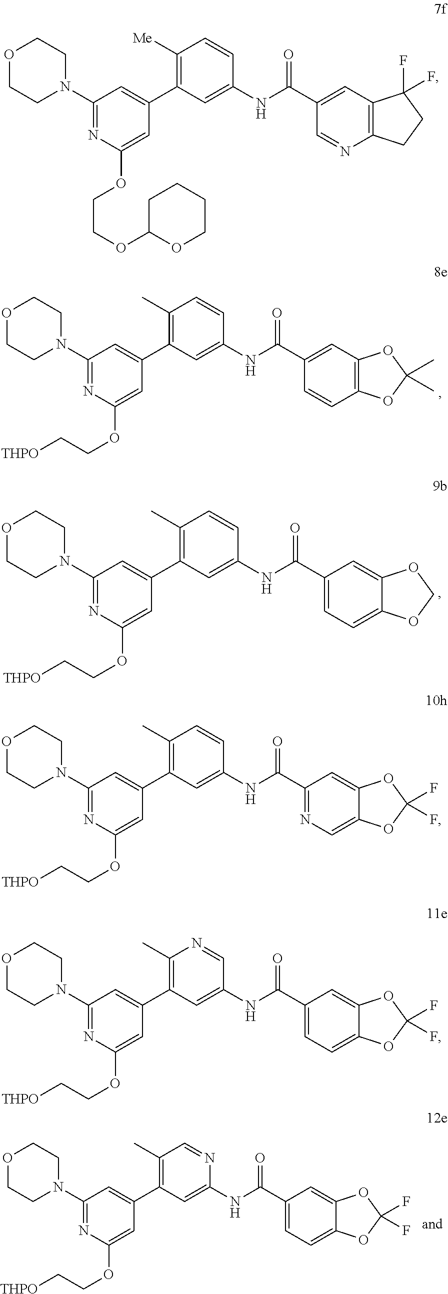

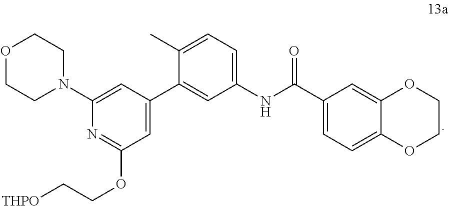

16. A pharmaceutical composition, comprising a compound of claim 1, or a tautomer, racemate, enantiomer, or diastereomer thereof, or a pharmaceutically acceptable salt, solvate or prodrug thereof, and a pharmaceutically acceptable carrier.

17. A method for treating a RAF-mediated disease or disorder, comprising administering to a subject in need thereof a therapeutically effective amount of a compound of claim 1, or a tautomer, racemate, enantiomer, or diastereomer thereof, or a pharmaceutically acceptable salt, solvate or prodrug thereof.

18. The method of claim 17, wherein the disease or disorder is a cancer selected from lymphoma, leukemia, breast cancer, lung cancer, prostate cancer, ovarian cancer, liver cancer, melanoma, rhabdomyosarcoma, synovial sarcoma, mesothelioma, cervical cancer, colon cancer, rectal cancer, stomach cancer, pancreatic cancer, brain cancer, skin cancer, oral cancer, bone cancer, kidney cancer, bladder cancer, fallopian tube tumor, ovarian tumor, peritoneal tumor, glioma, glioblastoma, head and neck cancer, and myeloma; preferably lymphoma, leukemia, breast cancer, lung cancer, prostate cancer, ovarian cancer, liver cancer, melanoma, rhabdomyosarcoma, synovial sarcoma, and mesothelioma.

19. A method for treating a RAF-associated cancer, comprising administering to a subject in need thereof a therapeutically effective amount of the pharmaceutical composition of claim 16, wherein the cancer is selected from lymphoma, leukemia, breast cancer, lung cancer, prostate cancer, ovarian cancer, liver cancer, melanoma, rhabdomyosarcoma, synovial sarcoma, mesothelioma, cervical cancer, colon cancer, rectal cancer, stomach cancer, pancreatic cancer, brain cancer, skin cancer, oral cancer, bone cancer, kidney cancer, bladder cancer, fallopian tube tumor, ovarian tumor, peritoneal tumor, glioma, glioblastoma, head and neck cancer, and myeloma; preferably lymphoma, leukemia, breast cancer, lung cancer, prostate cancer, ovarian cancer, liver cancer, melanoma, rhabdomyosarcoma, synovial sarcoma, and mesothelioma.

20. A process of preparing the compound of formula (III) according to claim 4, or a tautomer, racemate, enantiomer, or diastereomer thereof, or a pharmaceutically acceptable salt, solvate, or prodrug thereof, comprising a step of: 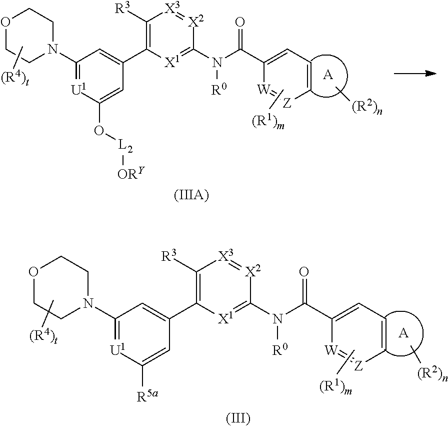
removing the hydroxyl protecting group of Formula (IIIA) to obtain the compound of formula (III), wherein:
L₂ is alkylene;
R^{Y} is a hydroxyl protecting group;
R^{5a} is —O-L₂-OH; and
W, Z, R⁰, X¹, X², X³, U¹, ring A, R¹ to R⁴, m, n and t are as defined in claim 4.
